SEQUENCE LISTING (5 sequences)

SEQ ID NO: 1 - protein, 443 aa, synthetic construct
  REGION 1..443: note = rHuPH20
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEP

SEQ ID NO: 2 - protein, 447 aa, synthetic construct [sequence omitted]

SEQ ID NO: 3 - protein, 446 aa, synthetic construct
  REGION 1..446: note = rHuPH20 variant 2
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIF

SEQ ID NO: 4 - protein, 445 aa, synthetic construct
  REGION 1..445: note = rHuPH20 variant 3
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQI

SEQ ID NO: 5 - protein, 444 aa, synthetic construct
  REGION 1..444: note = rHuPH20 variant 4
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQ

(19) United States
(12) Patent Application Publication
(10) Pub. No.: US 2025/0275967 A1
(43) Pub. Date: Sep. 4, 2025

(54) FREEZE DRIED COMPOSITIONS

(71) Applicant: Janssen Sciences Ireland Unlimited Company, Co Cork (IE)

(72) Inventors: Simone Vecchi, Vosselaar (BE); Miriam Colombo, Ulm (DE); Ann Gilberte P. De Saeger, Lint (BE); Maxim Paul M. Verstraeten, Melsele (BE); Iwan Caroline F. Vervoort, Hoogstraten (BE)

(73) Assignee: Janssen Sciences Ireland Unlimited Company, Co Cork (IE)

(21) Appl. No.: 18/858,037

(22) PCT Filed: Apr. 24, 2023
(86) PCT No.: PCT/EP2023/060686

§ 371 Date: Oct. 18, 2024

Related U.S. Application Data
(60) Provisional application No. 63/333,557, filed on Apr. 22, 2022.

(30) Foreign Application Priority Data
May 17, 2022 (EP) 22173920.4

(51) Int. Cl. A61K 31/505; A61K 9/19; A61K 38/47

(52) U.S. Cl. CPC A61K 31/505; A61K 9/19; A61K 38/47

(57) ABSTRACT

The invention relates to a solid composition obtainable by freeze drying an aqueous composition comprising rilpivirine or a pharmaceutically acceptable salt thereof and optionally a hyaluronidase. The invention also relates to a reconstituted aqueous composition obtainable by reconstituting the solid composition of the invention, a process for making the solid composition of the invention, and use of the reconstituted aqueous composition in the treatment or prevention of a HIV infection in a subject.

### CROSS REFERENCE TO RELATED APPLICATIONS

This application claims priority to U.S. Provisional Patent Application No. 63/333,557 filed on Apr. 22, 2022 and to European Patent Application No. EP22173920.4, filed on May 17, 2022, the entire contents of both of which are expressly incorporated herein by reference in their entirety.

### TECHNICAL FIELD

The present invention relates to a solid composition obtainable by freeze drying an aqueous composition comprising rilpivirine or a pharmaceutically acceptable salt thereof and optionally a hyaluronidase. The present invention also relates to a reconstituted aqueous composition obtainable by reconstituting the solid composition of the invention, a process for making the solid composition of the invention, and use of the reconstituted aqueous composition in the treatment or prevention of a HIV infection in a subject.

### BACKGROUND AND RELATED ART

The treatment of human immunodeficiency virus (HIV) infection, known as the cause of the acquired immunodeficiency syndrome (AIDS), remains a major medical challenge. HIV is able to evade immunological pressure, to adapt to a variety of cell types and growth conditions and to develop resistance against anti-HIV drugs. The latter include nucleoside reverse transcriptase inhibitors (NRTIs), non-nucleoside reverse transcriptase inhibitors (NNRTIs), nucleotide reverse transcriptase inhibitors (NtRTIs), HIV-protease inhibitors (PIs), integrase strand transfer inhibitors (INSTIs) and HIV fusion inhibitors. Currently available oral therapies require at least once daily dosing. Hence people living with HIV are reminded on a daily basis of their HIV-positive status and daily dosing may also lead to disclosure of their HIV-positive status. Daily dosing requires storage and transport of a large number or volume of pills and there remains the risk of patients forgetting to take their daily dose, thereby failing to comply with the prescribed dosage regimen. As well as reducing the effectiveness of the treatment, this also leads to the emergence of viral resistance.

One class of HIV drugs often used in highly active antiretroviral therapy (HAART) is the NNRTIs. Rilpivirine is an anti-retroviral of the NNRTI class that is used for the treatment of HIV infection. Rilpivirine is a second-generation NNRTI with higher potency and a reduced side effect profile compared with older NNRTIs. Rilpivirine activity is mediated by non-competitive inhibition of HIV-1 reverse transcriptase.

Rilpivirine not only shows pronounced activity against wild type HIV, but also against many of its mutated variants. Rilpivirine, its pharmacological activity, as well as a number of procedures for its preparation have been described in WO2003/016306.

Rilpivirine has been approved for the treatment of HIV infection and is commercially available as a single agent tablet (EDURANT®) containing 25 mg of rilpivirine base equivalent per tablet for once-daily oral administration as well as single tablet regimens for once-daily oral administration (COMPLERA®, ODEFSEY®, JULUCA®).

WO2007/147882 discloses intramuscular or subcutaneous injection of a therapeutically effective amount of rilpivirine in micro- or nanoparticle form, having a surface modifier adsorbed to the surface thereof; and a pharmaceutically acceptable aqueous carrier; wherein the rilpivirine active ingredient is suspended.

A prolonged release suspension for injection of rilpivirine for administration in combination with a prolonged release suspension for injection of cabotegravir has been approved as CABENUVA® in e.g. US and Canada, and as REKAMBYS® in e.g. the EU. These are the first anti-retrovirals to be provided in a long-acting injectable formulation for administration at intervals of greater than one day.

It is desirable to provide compositions comprising rilpivirine particles that can be stored for a long time-period (e.g. many weeks, months or years), in particular at room temperature, e.g. at 20-25° C., without substantially affecting the particle size distribution of the rilpivirine, so that when the rilpivirine is administered after storing for a long time-period, in particular at room temperature, e.g. at 20-25° C., bioavailability, efficacy and prolonged release properties are maintained over the storage period.

When rilpivirine is to be administered by subcutaneous or intramuscular injection it may also be desirable to formulate it or to administer it with a hyaluronidase to increase dispersion and absorption of the rilpivirine. Hyaluronidase may also be used to achieve other effects—for example, the administration of a hyaluronidase may reduce the bump formed by the administration of high volumes of a pharmaceutical composition at injection sites. However, storing hyaluronidases in pharmaceutical compositions, for example compositions comprising rilpivirine, for many weeks, months or years represents a significant challenge. When stored at room temperature for extended time-periods, hyaluronidases may unfold and degrade rapidly. Thus, it is also desirable to provide compositions comprising rilpivirine and a hyaluronidase, in which the hyaluronidase is stable during storage for many weeks, months or years, in particular at room temperature, e.g. at 20-25° C.

PCT/US2021/072453 (WO2022/109555) discloses the treatment or prevention of HIV infection using rilpivirine or a pharmaceutically acceptable salt thereof in the form of micro- or nanoparticles in suspension in combination with a hyaluronidase.

### SUMMARY OF THE INVENTION

In a first aspect the invention relates to a solid composition obtainable by freeze drying (synonymous with “lyophilising”) an aqueous composition comprising rilpivirine or a pharmaceutically acceptable salt thereof and a hyaluronidase.

In a second aspect the invention relates to a reconstituted aqueous composition obtainable by reconstituting the solid composition according to the first aspect.

In a third aspect there is provided a method for the treatment or prevention of HIV infection in a subject, the method comprising administering to the subject the reconstituted aqueous composition according to the second aspect.

In a fourth aspect there is provided a reconstituted aqueous composition according to the second aspect for use in the treatment or prevention of HIV infection in a subject.

In a fifth aspect there is provided the use of the reconstituted aqueous composition according to the second aspect for the manufacture of a medicament for treating or preventing HIV infection in a subject.

In a sixth aspect there is provided a solid composition according to the first aspect for use in the treatment or prevention of HIV infection in a subject.

In a seventh aspect there is provided the use of the solid composition according to the first aspect for the manufacture of a medicament for treating or preventing HIV infection in a subject.

In an eighth aspect there is provided a kit comprising (i) the solid composition according to the first aspect, and (ii) a diluent.

In a ninth aspect there is provided a kit comprising: (i) the solid composition according to the first aspect, (ii) a composition comprising one or more other antiretroviral agents, and optionally (iii) a diluent. In a tenth aspect there is provided a kit comprising: (i) the solid composition according to the first aspect, (ii) a composition comprising one or more other antiretroviral agents, and (iii) a diluent.

### BRIEF DESCRIPTION OF THE FIGURES

The invention will be described, by way of example only, with reference to the accompanying figures.

FIG. 1: Hyaluronidase melting temperature studies under different storage conditions

FIG. 1A: Hyaluronidase melting temperature studies under different storage conditions

FIG. 1B: Hyaluronidase melting temperature studies under different storage conditions

FIG. 1C: Hyaluronidase melting temperature studies under different storage conditions—pH

FIG. 2: Hyaluronidase melting temperature studies

FIG. 3: Hyaluronidase melting temperature studies under different storage conditions

FIG. 3A: Hyaluronidase melting temperature studies under different storage conditions—pH

FIG. 4: Hyaluronidase melting temperature studies and sodium CMC concentration

FIG. 5: Rilpivirine particle size under different storage conditions

FIG. 6: Rilpivirine particle size and hyaluronidase stability studies under different storage conditions

FIG. 7: Viscosity studies

FIG. 8: Injection force studies

These figures are explained further in the “Examples” section.

### DISCLOSURE OF THE INVENTION

This application has been drafted in sections to aid readability. However, this does not mean that each section is to be read in isolation. To the contrary, unless otherwise specified, each section is to be read with cross-referencing to the other sections, i.e. taking the entire application as a whole. No artificial separation of embodiments is intended, unless explicitly stated.

### DETAILED DESCRIPTION OF THE INVENTION

### The Compositions of the Invention

In a first aspect the invention relates to a solid composition obtainable by freeze drying an aqueous composition comprising rilpivirine or a pharmaceutically acceptable salt thereof and a hyaluronidase.

In a second aspect the invention relates to a reconstituted aqueous composition obtainable by reconstituting the solid composition according to the first aspect.

Freeze drying is a process in which an aqueous composition is initially frozen. During the initial freezing step, the water forms solid ice crystals and the composition concentrates leading to the separation of two phases. The pressure is then reduced during a primary drying stage, and the ice is sublimed. The temperature may be increased in the primary drying stage to increase the rate of sublimation. In a secondary drying stage, the temperature of the composition is increased slowly to promote the removal of residual water and provide a solid composition. Solid compositions obtainable by freeze drying can be referred to by multiple terms, including “lyophilised powder” “lyophilised cake”, “cake”, “freeze dried cake”, “freeze dried powder” and “freeze dried product”. In an embodiment, the solid composition obtainable by freeze drying is a lyophilized cake, or cake or freeze-dried cake.

Rilpivirine (4-[[4-[[4-[(1E)-2-cyanoethenyl]-2,6-dimethylphenyl]amino]-2-pyrimidinyl]amino]-benzonitrile; TMC278) has the following structural formula:

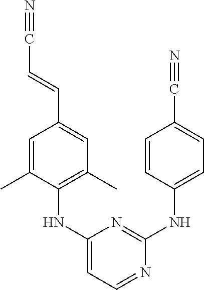 [SMILES: Cc1cc(/C=C/C#N)cc(C)c1Nc1ccnc(Nc2ccc(C#N)cc2)n1]

By “rilpivirine” it is meant rilpivirine having the structural formula shown above, i.e. the free base form.

In a preferred embodiment the aqueous composition or the reconstituted aqueous composition comprises rilpivirine, i.e. rilpivirine in its free base form. Pharmaceutically acceptable salts of rilpivirine means those where the counterion is pharmaceutically acceptable. The pharmaceutically acceptable salts are meant to comprise the therapeutically active non-toxic acid addition salt forms which rilpivirine is able to form. These salt forms can conveniently be obtained by treating rilpivirine with such appropriate acids as inorganic acids, for example, hydrohalic acids, e.g. hydrochloric, hydrobromic and the like; sulfuric acid; nitric acid; phosphoric acid and the like; or organic acids, for example, acetic, propanoic, hydroxyacetic, 2-hydroxypropanoic, 2-oxopropanoic, oxalic, malonic, succinic, maleic, fumaric, malic, tartaric, 2-hydroxy-1,2,3-propane-tricarboxylic, methanesulfonic, ethanesulfonic, benzenesulfonic, 4-methylbenzenesulfonic, cyclohexanesulfamic, 2-hydroxybenzoic, 4-amino-2-hydroxybenzoic and the like acids.

In an embodiment, the rilpivirine or a pharmaceutically acceptable salt thereof is in the form of particles suspended in the aqueous composition or the reconstituted aqueous composition, for example micro- or nanoparticles suspended in the aqueous composition or the reconstituted aqueous composition.

Two embodiments having preferred particle sizes are contemplated herein.

In the first preferred particle size embodiment, the rilpivirine particles have a Dᵥ90 of less than or about 2 μm. In this embodiment, the particles may have a Dᵥ90 of from about 100 nm to about 2 μm. In this embodiment, the particles may have a Dᵥ90 of from about 200 nm to about 2 μm, for example, of from 200 nm to about 2 μm. In this embodiment, the particles may have a Dᵥ90 of from 300 nm to about 2 μm. In this embodiment, the particles may have a Dᵥ90 of from about 400 nm to about 2 μm, for example, of from 400 nm to about 2 μm. In this embodiment, the particles may have a Dᵥ90 of from about 500 nm to about 2 μm, for example, of from 500 nm to about 2 μm. Preferably in this embodiment, the particles have a Dᵥ90 of from about 500 nm to about 1,600 nm, for example, of from 500 nm to about 1,600 nm or a Dᵥ90 of from about 500 nm to about 1,000 nm, for example, of from 500 nm to about 1,000 nm, for example about 800 nm. More preferably in this embodiment, the particles have a Dᵥ90 of about 500 nm to about 700 nm, even more preferably from about 500 nm to about 650 nm, and most preferably from about 525 nm to about 644 nm.

The term “Dᵥ90” as used herein refers to the diameter below which 90% by volume of the particle population is found. The term “Dᵥ50” as used herein refers to the diameter below which 50% by volume of the particle population is found. The term “Dᵥ10” as used herein refers to the diameter below which 10% by volume of the particle population is found.

In the first preferred particle size embodiment, the particles may have a Dᵥ50 of less than or about 1,000 nm. In this embodiment, the particles may have a Dᵥ50 of from about 10 nm to about 1,000 nm. In this embodiment, the particles may have a Dᵥ50 of from about 50 nm to about 700 nm. In this embodiment, the particles may have a Dᵥ50 of from about 100 nm to about 600 nm. In this embodiment, the particles may have a Dᵥ50 of from about 150 nm to about 500 nm. Preferably in this embodiment, the particles have a Dᵥ50 of from about 200 nm to about 500 nm.

In the first preferred particle size embodiment, the particles may have a Dᵥ10 of less than or about 500 nm. In this embodiment, the particles may have a Dᵥ10 of from about 10 nm to about 500 nm. In this embodiment, the particles may have a Dᵥ10 of from about 25 nm to about 400 nm. In this embodiment, the particles may have a Dᵥ10 of from about 50 nm to about 300 nm. In this embodiment, the particles may have a Dᵥ10 of from about 50 nm to about 200 nm. Preferably in this embodiment, the particles have a Dᵥ10 of from about 75 nm to about 200 nm.

Preferably in this embodiment, the particles have a Dᵥ90 of from about 500 nm to about 1,600 nm, a Dᵥ50 of from about 200 nm to about 500 nm and a Dᵥ10 of from about 75 nm to about 200 nm.

Alternatively, the particles have a Dᵥ90 of from about 500 nm to about 1,000 nm, a Dᵥ50 of from about 200 nm to about 500 nm and a Dᵥ10 of from about 75 nm to about 200 nm.

Alternatively, the particles have a Dᵥ90 of from about 500 nm to about 700 nm, a Dᵥ50 of from about 200 nm to about 500 nm and a Dᵥ10 of from about 75 nm to about 200 nm.

In the second preferred particle size embodiment, the particles may have a Dᵥ90 of from about 1 μm to about 10 μm. In this embodiment, the particles may have a Dᵥ90 of from about 2 μm to about 9 μm. In this embodiment, the particles may have a Dᵥ90 of from about 3 μm to about 8 μm. In this embodiment, the particles may have a Dᵥ90 of from about 3 μm to about 7 μm. Preferably in this embodiment, the particles have a Dᵥ90 of from about 4 μm to about 6 μm. Most preferably in this embodiment, the particles have a Dᵥ90 of about 5 μm to about 6 μm, e.g. about 5 μm or about 6 μm.

In the second preferred particle size embodiment, the particles have a Dᵥ50 of less than or about 3 μm. In this embodiment, the particles may have a Dᵥ50 of less than about 2.5 μm. In this embodiment, the particles may have a Dᵥ50 of from about 1 μm to about 2.5 μm. In this embodiment, the particles may have a Dᵥ50 of from about 1.2 μm to about 2.2 μm. Preferably in this embodiment, the particles have a Dᵥ50 of from about 1.5 μm to about 2.2 μm. Further preferably in this embodiment, the particles have a Dᵥ50 of from about 1.5 μm to about 2 μm.

In the second preferred particle size embodiment, the particles may have a Dᵥ10 of less than or about 1000 nm. In this embodiment, the particles may have a Dᵥ10 of from about 10 nm to about 1000 nm. In this embodiment, the particles may have a Dᵥ10 of from about 100 nm to about 700 nm. In this embodiment, the particles may have a Dᵥ10 of from about 200 nm to about 600 nm. Preferably in this embodiment, the particles have a Dᵥ10 of from about 300 nm to about 500 nm.

Preferably in this embodiment, the particles have a Dᵥ90 of from about 4 μm to about 6 μm, a Dᵥ50 of from about 1.5 μm to about 2 μm and a Dᵥ10 of from about 300 nm to about 500 nm.

Preferably in this embodiment, the particles have a Dᵥ90 of from about 5 μm to about 6 μm, a Dᵥ50 of from about 1.5 μm to about 2.2 μm and a Dᵥ10 of from about 300 nm to about 500 nm.

The Dᵥ10, Dᵥ50 and Dᵥ90 as used herein are determined by routine laser diffraction techniques, e.g. in accordance with ISO 13320:2009.

Laser diffraction relies on the principle that a particle will scatter light at an angle that varies depending on the size the particle and a collection of particles will produce a pattern of scattered light defined by intensity and angle that can be correlated to a particle size distribution. A number of laser diffraction instruments are commercially available for the rapid and reliable determination of particle size distributions. For example, particle size distribution may be measured by the conventional Malvern Mastersizer™ 3000 particle size analyser from Malvern Instruments. The Malvern Mastersizer™ 3000 particle size analyser operates by projecting a helium-neon gas laser beam through a transparent cell containing the particles of interest suspended in an aqueous solution. Light rays which strike the particles are scattered through angles which are inversely proportional to the particle size and a photodetector array measures the intensity of light at several predetermined angles and the measured intensities at different angles are processed by a computer using standard theoretical principles to determine the particle size distribution. Laser diffraction values may be obtained using a wet dispersion of the particles in distilled water.

Other methods that are commonly used in the art to measure Dᵥ10, Dᵥ50 and Dᵥ90 include disc centrifugation, scanning electron microscope (SEM), sedimentation field flow fractionation and photon correlation spectroscopy.

In an embodiment, the aqueous composition or the reconstituted aqueous composition comprises from about 100 to about 500 mg/mL rilpivirine or a pharmaceutically acceptable salt thereof. In an embodiment, the aqueous composition or the reconstituted aqueous composition comprises from about 150 to about 450 mg/mL rilpivirine or a pharmaceutically acceptable salt thereof. In an embodiment, the aqueous composition or the reconstituted aqueous composition comprises from about 200 to about 400 mg/mL rilpivirine or a pharmaceutically acceptable salt thereof. In a preferred embodiment, the aqueous composition or the reconstituted aqueous composition comprises from about 250 to about 350 mg/mL rilpivirine or a pharmaceutically acceptable salt thereof, e.g. about 300 mg/mL, in particular 300 mg/mL of rilpivirine.

In an embodiment, the amount of the rilpivirine or pharmaceutically acceptable salt thereof in the solid composition or the reconstituted aqueous composition is from about 900 mg to about 28800 mg (e.g. from about 900 mg to about 14400 mg, or from about 900 mg to about 7200 mg, or from about 900 mg to about 4500 mg, or from about 900 mg to about 3600 mg), preferably from about 1200 mg to about 14400 mg, preferably from about 1350 mg to about 13200 mg, preferably from about 1500 mg to about 12000 mg, (e.g. from about 3000 mg to about 12000 mg), preferably from about 1800 mg to about 10800 mg (e.g. from about 2700 mg to about 10800 mg, or from about 1800 mg to about 3600 mg), most preferably from about 1800 mg to about 7200 mg, or from about 1800 mg to about 4500 mg, or from about 2700 mg to about 4500 mg, or from about 3600 mg to about 4500 mg. The indicated “mg” corresponds to mg of rilpivirine (i.e. rilpivirine in its free base form). Thus, by way of example, 1 mg of rilpivirine (i.e. rilpivirine in its free base form) corresponds to 1.1 mg of rilpivirine hydrochloride.

The solid composition or the reconstituted aqueous composition comprises a hyaluronidase. A hyaluronidase is an enzyme that degrades hyaluronic acid (HA) e.g. in the skin and lowers the viscosity of hyaluronan in the extracellular matrix. Because of this property, it can be used to increase dispersion and absorption of injected active pharmaceutical ingredients and/or to enable administration of larger volumes subcutaneously. Enzymatic activity of hyaluronidase, including rHuPH20, can be defined by units per mL (U/mL) or by total enzyme activity in a particular formulation (U).

The term “hyaluronidase” as used herein means any enzyme that degrades hyaluronic acid and lowers the viscosity of hyaluronan in the extracellular matrix.

In an embodiment, the hyaluronidase is recombinant hyaluronidase. In a preferred embodiment, the hyaluronidase is recombinant human hyaluronidase, e.g. rHuPH20. In an embodiment, rHuPH20 is defined by the amino acid sequence available under CAS Registry No. 757971-58-7. Further information regarding rHuPH20 is provided in Int. Pat. Publ. No. WO2004/078140. In an embodiment, the amino acid sequence of rHuPH20 comprises SEQ ID NO: 1. In some embodiments, the hyaluronidase is a variant of rHuPH20 having an amino acid sequence of rHuPH20 that comprises SEQ ID NO: 2, namely residues 36-482 of wild type human hyaluronidase. In some embodiments, the hyaluronidase is a variant of rHuPH2O having an amino acid sequence that comprises SEQ ID NO: 3. In some embodiments, the hyaluronidase is a variant of rHuPH2O having an amino acid sequence that comprises SEQ ID NO: 4. In some embodiments, the hyaluronidase is a variant of rHuPH2O having an amino acid sequence that comprises SEQ ID NO: 5.

| | |
|---|---|
| SEQ ID NO: 1: | LNFRAPPVIPNVPFLWAWNAPSEFCLGKFDEPLDMSLFSFIGSPRIN |
| rHuPH20 | ATGQGVTIFYVDRLGYYPYIDSITGVTVNGGIPQKISLQDHLDKAK |
| | KDITFYMPVDNLGMAVIDWEEWRPTWARNWKPKDVYKNRSIEL |
| | VQQQNVQLSLTEATEKAKQEFEKAGKDFLVETIKLGKLLRPNHLW |
| | GYYLFPDCYNHHYKKPGYNGSCFNVEIKRNDDLSWLWNESTALY |
| | PSIYLNTQQSPVAATLYVRNRVREAIRVSKIPDAKSPLPVFAYTRIV |
| | FTDQVLKFLSQDELVYTFGETVALGASGIVIWGTLSIMRSMKSCLL |
| | LDNYMETILNPYIINVTLAAKMCSQVLCQEQGVCIRKNWNSSDYL |
| | HLNPDNFAIQLEKGGKFTVRGKPTLEDLEQFSEKFYCSCYSTLSCK |
| | EKADVKDTDAVDVCIADGVCIDAFLKPPMETEEP |
| SEQ ID NO: 2: | LNFRAPPVIPNVPFLWAWNAPSEFCLGKFDEPLDMSLFSFIGSPRIN |
| rHuPH20 variant 1 | ATGQGVTIFYVDRLGYYPYIDSITGVTVNGGIPQKISLQDHLDKAK |
| | KDITFYMPVDNLGMAVIDWEEWRPTWARNWKPKDVYKNRSIEL |
| | VQQQNVQLSLTEATEKAKQEFEKAGKDFLVETIKLGKLLRPNHLW |
| | GYYLFPDCYNHHYKKPGYNGSCFNVEIKRNDDLSWLWNESTALY |
| | PSIYLNTQQSPVAATLYVRNRVREAIRVSKIPDAKSPLPVFAYTRIV |
| | FTDQVLKFLSQDELVYTFGETVALGASGIVIWGTLSIMRSMKSCLL |
| | LDNYMETILNPYIINVTLAAKMCSQVLCQEQGVCIRKNWNSSDYL |
| | HLNPDNFAIQLEKGGKFTVRGKPTLEDLEQFSEKFYCSCYSTLSCK |
| | EKADVKDTDAVDVCIADGVCIDAFLKPPMETEEPQIFY |
| SEQ ID NO: 3: | LNFRAPPVIPNVPFLWAWNAPSEFCLGKFDEPLDMSLFSFIGSPRIN |
| rHuPH20 variant 2 | ATGQGVTIFYVDRLGYYPYIDSITGVTVNGGIPQKISLQDHLDKAK |
| | KDITFYMPVDNLGMAVIDWEEWRPTWARNWKPKDVYKNRSIEL |
| | VQQQNVQLSLTEATEKAKQEFEKAGKDFLVETIKLGKLLRPNHLW |
| | GYYLFPDCYNHHYKKPGYNGSCFNVEIKRNDDLSWLWNESTALY |
| | PSIYLNTQQSPVAATLYVRNRVREAIRVSKIPDAKSPLPVFAYTRIV |
| | FTDQVLKFLSQDELVYTFGETVALGASGIVIWGTLSIMRSMKSCLL |
| | LDNYMETILNPYIINVTLAAKMCSQVLCQEQGVCIRKNWNSSDYL |
| | HLNPDNFAIQLEKGGKFTVRGKPTLEDLEQFSEKFYCSCYSTLSCK |
| | EKADVKDTDAVDVCIADGVCIDAFLKPPMETEEPQIF |
| SEQ ID NO: 4: | LNFRAPPVIPNVPFLWAWNAPSEFCLGKFDEPLDMSLFSFIGSPRIN |
| rHuPH20 variant 3 | ATGQGVTIFYVDRLGYYPYIDSITGVTVNGGIPQKISLQDHLDKAK |
| | KDITFYMPVDNLGMAVIDWEEWRPTWARNWKPKDVYKNRSIEL |
| | VQQQNVQLSLTEATEKAKQEFEKAGKDFLVETIKLGKLLRPNHLW |
| | GYYLFPDCYNHHYKKPGYNGSCFNVEIKRNDDLSWLWNESTALY |
| | PSIYLNTQQSPVAATLYVRNRVREAIRVSKIPDAKSPLPVFAYTRIV |
| | FTDQVLKFLSQDELVYTFGETVALGASGIVIWGTLSIMRSMKSCLL |
| | LDNYMETILNPYIINVTLAAKMCSQVLCQEQGVCIRKNWNSSDYL |
| | HLNPDNFAIQLEKGGKFTVRGKPTLEDLEQFSEKFYCSCYSTLSCK |
| | EKADVKDTDAVDVCIADGVCIDAFLKPPMETEEPQI |
| SEQ ID NO: 5: | LNFRAPPVIPNVPFLWAWNAPSEFCLGKFDEPLDMSLFSFIGSPRIN |
| rHuPH20 variant 4 | ATGQGVTIFYVDRLGYYPYIDSITGVTVNGGIPQKISLQDHLDKAK |
| | KDITFYMPVDNLGMAVIDWEEWRPTWARNWKPKDVYKNRSIEL |
| | VQQQNVQLSLTEATEKAKQEFEKAGKDFLVETIKLGKLLRPNHLW |
| | GYYLFPDCYNHHYKKPGYNGSCFNVEIKRNDDLSWLWNESTALY |
| | PSIYLNTQQSPVAATLYVRNRVREAIRVSKIPDAKSPLPVFAYTRIV |
| | FTDQVLKFLSQDELVYTFGETVALGASGIVIWGTLSIMRSMKSCLL |
| | LDNYMETILNPYIINVTLAAKMCSQVLCQEQGVCIRKNWNSSDYL |
| | HLNPDNFAIQLEKGGKFTVRGKPTLEDLEQFSEKFYCSCYSTLSCK |
| | EKADVKDTDAVDVCIADGVCIDAFLKPPMETEEPQ |

In an embodiment, the concentration of the hyaluronidase in the aqueous composition or the reconstituted aqueous composition is from about 10 to about 10,000 U/mL, or from about 100 to about 10,000 U/mL, or from about 500 to about 10,000 U/mL, or from about 500 to about 5,000 U/mL, or from about 500 to about 2500 U/mL, or from about 1000 to about 2500 U/mL, or from about 1500 to about 2500 U/mL. Preferably, the concentration of the hyaluronidase in the aqueous composition or the reconstituted aqueous composition is from about 1800 to about 2200 U/mL (e.g. about 2000 U/mL).

In an embodiment, the concentration of the hyaluronidase in the aqueous composition or the reconstituted aqueous composition is from about 0.1 to about 90 μg/mL, or from about 0.9 to about 90 μg/mL, or from about 4.5 to about 90 μg/mL, or from about 4.5 to about 45 μg/mL, or from about 4.5 to about 22.5 μg/mL, or from about 9 to about 22.5 μg/mL, or from about 13.5 to about 22.5 μg/mL. Preferably, the concentration of the hyaluronidase in the aqueous composition or the reconstituted aqueous composition is from about 16.2 to about 19.8 μg/mL, (e.g. about 18 μg/mL).

In an embodiment, the aqueous composition (and, as a consequence, the solid composition) or the reconstituted aqueous composition additionally comprises a carboxymethyl cellulose (CMC) or a pharmaceutically acceptable salt thereof, preferably wherein the CMC is not cross-linked. In an embodiment, the CMC or a pharmaceutically acceptable salt thereof is a pharmaceutically acceptable salt of CMC. Pharmaceutically acceptable salts of CMC means those where the counterion is pharmaceutically acceptable. The pharmaceutically acceptable salts are meant to comprise the therapeutically active non-toxic base addition salt forms which CMC is able to form. Preferred pharmaceutically acceptable salts of CMC include sodium CMC and potassium CMC. In a particularly preferred embodiment, the CMC or a pharmaceutically acceptable salt thereof is sodium CMC, particularly non-cross-linked sodium CMC. In another embodiment, the CMC or a pharmaceutically acceptable salt thereof is CMC. Examples of CMCs that may be used in the invention are carmellose sodium, 40 mPa·s (parenteral grade), available from Ashland, and Blanose CMC 7LF PH or Blanose 7LP EP, available from Ashland.

The CMC or a pharmaceutically acceptable salt thereof can have any degree of substitution (DS). The DS is the average number of carboxymethyl groups per cellulose unit. In a preferred embodiment, the DS is from about 0.4 to about 1.5. In an embodiment, the CMC or a pharmaceutically acceptable salt thereof has a DS of from about 0.5 to about 1, for example from about 0.65 to about 0.95 such as about 0.7.

In an embodiment, the CMC or a pharmaceutically acceptable salt thereof has a viscosity of from about 10 mPa·s to about 100 mPa·s in an aqueous solution of 1% (w/v) at room temperature. In an embodiment, the CMC or a pharmaceutically acceptable salt thereof has a viscosity of from about 20 mPa·s to about 60 mPa·s in an aqueous solution of 1% (w/v) at room temperature. In an embodiment, the CMC or a pharmaceutically acceptable salt thereof has a viscosity of from about 30 mPa·s to about 50 mPa·s in an aqueous solution of 1% (w/v) at room temperature, for example about 40 mPa·s in an aqueous solution of 1% (w/v) at room temperature.

In an embodiment, the CMC or a pharmaceutically acceptable salt thereof has a viscosity of from about 10 mPa·s to about 100 mPa·s in an aqueous solution of 2% (w/v) at room temperature. In an embodiment, the CMC or a pharmaceutically acceptable salt thereof has a viscosity of from about 20 mPa·s to about 60 mPa·s in an aqueous solution of 2% (w/v) at room temperature. In a preferred embodiment, the CMC or a pharmaceutically acceptable salt thereof has a viscosity of from about 30 mPa·s to about 50 mPa·s in an aqueous solution of 2% (w/v) at room temperature, for example about 40 mPa·s in an aqueous solution of 2% (w/v) at room temperature.

In an embodiment, the molecular weight of the CMC or a pharmaceutically acceptable salt thereof is from about 50 kDa to about 2,000 kDa. In an embodiment, the molecular weight of the CMC or a pharmaceutically acceptable salt thereof is from about 50 kDa to about 1,000 kDa. In an embodiment, the molecular weight of the CMC or a pharmaceutically acceptable salt thereof is from about 70 kDa to about 900 kDa. In a preferred embodiment, the molecular weight of the CMC or a pharmaceutically acceptable salt thereof is from about 90 kDa to about 750 kDa, In a most preferred embodiment, the molecular weight of the CMC or a pharmaceutically acceptable salt thereof is from about 70 kDa to about 110 kDa, for example about 90 kDa.

The inventors have surprisingly found that the addition of CMC or a pharmaceutically acceptable salt thereof to the aqueous compositions of the invention which comprise a hyaluronidase increases the melting temperature (Tₘ) of the hyaluronidase (Example 2a). This is indicative of improved stability on storage.

The inventors have also found that the addition of CMC or a pharmaceutically acceptable salt thereof to the aqueous compositions of the invention which comprise a hyaluronidase leads to a decrease in the loss of hyaluronidase activity, decrease in aggregation of the hyaluronidase, and/or decrease in oxidation of the hyaluronidase in the reconstituted aqueous composition of the invention, such as following storage of the freeze dried product under stress test conditions (Example 6 and 8). This is indicative of improved stability on storage.

In an embodiment, the aqueous composition or the reconstituted aqueous composition comprises from about 1 mg/mL to about 100 mg/mL of the CMC or a pharmaceutically acceptable salt thereof. In an embodiment, the aqueous composition or the reconstituted aqueous composition comprises from about 1 mg/mL to about 75 mg/mL of the CMC or a pharmaceutically acceptable salt thereof. In an embodiment, the aqueous composition or the reconstituted aqueous composition comprises from about 1 mg/mL to about 50 mg/mL of the CMC or a pharmaceutically acceptable salt thereof. In an embodiment, the aqueous composition or the reconstituted aqueous composition comprises from about 1 mg/mL to about 25 mg/mL of the CMC or a pharmaceutically acceptable salt thereof. In an embodiment, the aqueous composition or the reconstituted aqueous composition comprises from about 5 mg/mL to about 25 mg/mL of the CMC or a pharmaceutically acceptable salt thereof. In an embodiment, the aqueous composition or the reconstituted aqueous composition comprises from about 5 mg/mL to about 15 mg/mL of the CMC or a pharmaceutically acceptable salt thereof. In a preferred embodiment, the aqueous composition or the reconstituted aqueous composition comprises from about 5 mg/mL to about 10 mg/mL of the CMC or a pharmaceutically acceptable salt thereof. In a most preferred embodiment, the aqueous composition or the reconstituted aqueous composition comprises from about 1 mg/mL to about 5 mg/mL of the CMC or a pharmaceutically acceptable salt thereof, e.g. about 3 mg/mL.

In a preferred embodiment, the CMC or a pharmaceutically acceptable salt thereof is sodium CMC and the aqueous composition or the reconstituted aqueous composition comprises the sodium CMC in any one of the amounts specified in the paragraph directly above. In a preferred embodiment, the CMC or a pharmaceutically acceptable salt thereof is sodium CMC and the aqueous composition or the reconstituted aqueous composition has any of the degrees of substitution, viscosities and molecular weights specified herein.

In an embodiment, the aqueous composition (and, as a consequence, the solid composition) or the reconstituted aqueous composition comprises from about 0.002 mg to about 5 mg CMC or a pharmaceutically acceptable salt thereof per 100 U hyaluronidase. In an embodiment, the aqueous composition (and, as a consequence, the solid composition) or the reconstituted aqueous composition comprises from about 0.01 mg to about 5 mg CMC or a pharmaceutically acceptable salt thereof per 100 U hyaluronidase. In an embodiment, the aqueous composition (and, as a consequence, the solid composition) or the reconstituted aqueous composition comprises from about 0.05 mg to about 2.5 mg CMC or a pharmaceutically acceptable salt thereof per 100 U hyaluronidase. In an embodiment, the aqueous composition (and, as a consequence, the solid composition) or the reconstituted aqueous composition comprises from about 0.1 mg to about 2 mg CMC or a pharmaceutically acceptable salt thereof per 100 U hyaluronidase. In a preferred embodiment, the aqueous composition (and, as a consequence, the solid composition) or the reconstituted aqueous composition comprises from about 0.1 mg to about 1 mg CMC or a pharmaceutically acceptable salt thereof per 100 U hyaluronidase. In a more preferred embodiment, the aqueous composition (and, as a consequence, the solid composition) or the reconstituted aqueous composition comprises from about 0.1 mg to about 0.25 mg CMC or a pharmaceutically acceptable salt thereof per 100 U hyaluronidase, e.g. about 0.15 mg CMC or a pharmaceutically acceptable salt thereof per 100 U hyaluronidase. In another more preferred embodiment, the aqueous composition (and, as a consequence, the solid composition) or the reconstituted aqueous composition comprises from about 0.05 mg to about 0.25 mg CMC or a pharmaceutically acceptable salt thereof per 100 U hyaluronidase, e.g. about 0.15 mg CMC or a pharmaceutically acceptable salt thereof per 100 U hyaluronidase.

In a preferred embodiment, the CMC or a pharmaceutically acceptable salt thereof is sodium CMC and the aqueous composition (and, as a consequence, the solid composition) or the reconstituted aqueous composition comprises any one of the ratios of sodium CMC to hyaluronidase specified in the paragraph directly above.

In an embodiment, the aqueous composition (and, as a consequence, the solid composition) or the reconstituted aqueous composition additionally comprises a cryoprotectant. In an embodiment the cryoprotectant is a sugar, sugar alcohol or an amino acid or a pharmaceutically acceptable salt thereof, preferably wherein the sugar is not a monosaccharide. Pharmaceutically acceptable salts of a sugar, sugar alcohol or an amino acid means those where the counterion is pharmaceutically acceptable. The pharmaceutically acceptable salts are meant to comprise the therapeutically active non-toxic acid and base addition salt forms which a given sugar, sugar alcohol or an amino acid is able to form. Suitable sugars and sugar alcohols include mannitol, lactose, sucrose, trehalose, sorbitol, dextrose, fructose, maltose, xylitol and raffinose. Preferably, the sugar or sugar alcohol is selected from mannitol and sucrose. More preferably, the sugar or sugar alcohol is sucrose. Suitable amino acids or pharmaceutically acceptable salts thereof include arginine, glycine and histidine or a pharmaceutically acceptable salt thereof. In an embodiment, the amino acid or a pharmaceutically acceptable salt thereof is selected from arginine and glycine or a pharmaceutically acceptable salt thereof. In a preferred embodiment, the amino acid or a pharmaceutically acceptable salt thereof is arginine (e.g. arginine HCl). In an embodiment, the aqueous composition or the reconstituted aqueous composition additionally comprises two cryoprotectants. In an embodiment, the aqueous composition or the reconstituted aqueous composition additionally comprises two cryoprotectants, wherein one cryoprotectant is a sugar or sugar alcohol and the other cryoprotectant is an amino acid or a pharmaceutically acceptable salt thereof.

The inventors surprisingly found that when the rilpivirine or a pharmaceutically acceptable salt thereof is in the form of particles suspended in the aqueous composition, the particle size distribution in the reconstituted aqueous compositions according to the invention was similar to that in the aqueous composition prior to the freeze drying (see e.g. Example 4).

In an embodiment, the aqueous composition or the reconstituted aqueous composition comprises from about 1 mg/mL to about 200 mg/mL of the cryoprotectant. In an embodiment, the aqueous composition or the reconstituted aqueous composition comprises from about 1 mg/mL to about 175 mg/mL of the cryoprotectant. In an embodiment, the aqueous composition or the reconstituted aqueous composition comprises from about 1 mg/mL to about 150 mg/mL of the cryoprotectant.

In an embodiment, the aqueous composition or the reconstituted aqueous composition comprises from about 20 mg/mL to about 150 mg/mL of the cryoprotectant.

In an embodiment, the aqueous composition or the reconstituted aqueous composition comprises a cryoprotectant which is a sugar or sugar alcohol in an amount of from about 25 mg/mL to about 150 mg/mL. In an embodiment, the aqueous composition or the reconstituted aqueous composition comprises a cryoprotectant which is a sugar or sugar alcohol in an amount of from about 25 mg/mL to about 125 mg/mL. In a preferred embodiment, the aqueous composition or the reconstituted aqueous composition comprises a cryoprotectant which is a sugar or sugar alcohol in an amount of from about 50 mg/mL to about 100 mg/mL. In another preferred embodiment, the aqueous composition or the reconstituted aqueous composition comprises a cryoprotectant which is a sugar or sugar alcohol in an amount of from about 75 mg/mL to about 125 mg/mL, e.g. about 100 mg/mL. In an embodiment, the cryoprotectant is sucrose.

In an embodiment, the aqueous composition or the reconstituted aqueous composition comprises a cryoprotectant which is an amino acid or a pharmaceutically acceptable salt thereof in an amount of from about 1 mg/mL to about 100 mg/mL. In a preferred embodiment, the aqueous composition or the reconstituted aqueous composition comprises a cryoprotectant which is an amino acid or a pharmaceutically acceptable salt thereof in an amount of from about 25 mg/mL to about 75 mg/mL, e.g. about 50 mg/mL. In a more preferred embodiment, the aqueous composition or the reconstituted aqueous composition comprises a cryoprotectant which is an amino acid or a pharmaceutically acceptable salt thereof in an amount of from about 25 mg/mL to about 35 mg/mL, e.g. about 30 mg/mL. In an embodiment, the cryoprotectant is arginine, preferably arginine HCl.

In an embodiment, the aqueous composition or the reconstituted aqueous composition comprises two cryoprotectants, wherein the total amount of the two cryoprotectants is from about 25 mg/mL to about 150 mg/mL, preferably wherein the two cryoprotectants are a sugar or sugar alcohol and an amino acid. In a preferred embodiment, the aqueous composition or the reconstituted aqueous composition comprises two cryoprotectants, wherein the total amount of the two cryoprotectants is from about 50 mg/mL to about 125 mg/mL, preferably wherein the two cryoprotectants are a sugar or sugar alcohol and an amino acid, for example wherein the two cryoprotectants are a sugar or sugar alcohol and an amino acid or a pharmaceutically acceptable salt thereof.

In a preferred embodiment, the aqueous composition (and, as a consequence, the solid composition) or the reconstituted aqueous composition additionally comprises sucrose and CMC or a pharmaceutically acceptable salt thereof. In a more preferred embodiment, the aqueous composition (and, as a consequence, the solid composition) or the reconstituted aqueous composition additionally comprises sucrose and sodium CMC.

In a preferred embodiment, the aqueous composition (and, as a consequence, the solid composition) or the reconstituted aqueous composition additionally comprises arginine or a pharmaceutically acceptable salt thereof and CMC or a pharmaceutically acceptable salt thereof. In a more preferred embodiment, the aqueous composition (and, as a consequence, the solid composition) or the reconstituted aqueous composition additionally comprises arginine hydrochloride and sodium CMC.

In a preferred embodiment, the aqueous composition (and, as a consequence, the solid composition) or the reconstituted aqueous composition additionally comprises arginine or a pharmaceutically acceptable salt thereof. In a more preferred embodiment, the aqueous composition (and, as a consequence, the solid composition) or the reconstituted aqueous composition additionally comprises arginine hydrochloride.

In an embodiment, the ratio of rilpivirine or a pharmaceutically acceptable salt thereof to cryoprotectant in the aqueous composition (and, as a consequence, the solid composition) or the reconstituted aqueous composition is from about 1:10 (w/w) to about 10:1 (w/w).

In an embodiment, the ratio of rilpivirine or a pharmaceutically acceptable salt thereof to cryoprotectant in the aqueous composition (and, as a consequence, the solid composition) or the reconstituted aqueous composition is from about 1:1 (w/w) to about 20:1 (w/w). In an embodiment, the ratio of rilpivirine or a pharmaceutically acceptable salt thereof to cryoprotectant in the aqueous composition (and, as a consequence, the solid composition) or the reconstituted aqueous composition is from about 1:1 (w/w) to about 10:1 (w/w). In an embodiment, the ratio of rilpivirine or a pharmaceutically acceptable salt thereof to cryoprotectant in the aqueous composition (and, as a consequence, the solid composition) or the reconstituted aqueous composition is from about 2:1 (w/w) to about 10:1 (w/w). In an embodiment, the ratio of rilpivirine or a pharmaceutically acceptable salt thereof to cryoprotectant in the aqueous composition (and, as a consequence, the solid composition) or the reconstituted aqueous composition is from about 2:1 (w/w) to about 7:1 (w/w). In a preferred embodiment, the ratio of rilpivirine or a pharmaceutically acceptable salt thereof to cryoprotectant in the aqueous composition (and, as a consequence, the solid composition) or the reconstituted aqueous composition is from about 3:1 (w/w) to about 6:1 (w/w).

In a preferred embodiment, the ratio of rilpivirine or a pharmaceutically acceptable salt thereof to cryoprotectant in the aqueous composition (and, as a consequence, the solid composition) or the reconstituted aqueous composition is from about 1:1 (w/w) to about 15:1 (w/w). In a particularly preferred embodiment, the ratio of rilpivirine or a pharmaceutically acceptable salt thereof to cryoprotectant in the aqueous composition (and, as a consequence, the solid composition) or the reconstituted aqueous composition is from about 1:1 (w/w) to about 5:1 (w/w), for example about 3:1 (w/w). In another particularly preferred embodiment, the ratio of rilpivirine or a pharmaceutically acceptable salt thereof to cryoprotectant in the aqueous composition (and, as a consequence, the solid composition) or the reconstituted aqueous composition is from about 8:1 (w/w) to about 12:1 (w/w), for example about 10:1 (w/w).

In an embodiment, the ratio of rilpivirine or a pharmaceutically acceptable salt thereof to cryoprotectant in the aqueous composition (and, as a consequence, the solid composition) or the reconstituted aqueous composition is from about 1:1 (w/w) to about 1:10 (w/w). In an embodiment, the ratio of rilpivirine or a pharmaceutically acceptable salt thereof to cryoprotectant in the aqueous composition (and, as a consequence, the solid composition) or the reconstituted aqueous composition is from about 1:2 (w/w) to about 1:10 (w/w). In an embodiment, the ratio of rilpivirine or a pharmaceutically acceptable salt thereof to cryoprotectant in the aqueous composition (and, as a consequence, the solid composition) or the reconstituted aqueous composition is from about 1:2 (w/w) to about 1:7 (w/w). In a preferred embodiment, the ratio of rilpivirine or a pharmaceutically acceptable salt thereof to cryoprotectant in the aqueous composition (and, as a consequence, the solid composition) or the reconstituted aqueous composition is from about 1:3 (w/w) to about 1:6 (w/w).

In an embodiment, the ratio of CMC or a pharmaceutically acceptable salt thereof to cryoprotectant in the aqueous composition (and, as a consequence, the solid composition) or the reconstituted aqueous composition is from about 1:1 (w/w) to about 1:150 (w/w). In an embodiment, the ratio of CMC or a pharmaceutically acceptable salt thereof to cryoprotectant in the aqueous composition (and, as a consequence, the solid composition) or the reconstituted aqueous composition is from about 1:1 (w/w) to about 1:100 (w/w). In an embodiment, the ratio of CMC or a pharmaceutically acceptable salt thereof to cryoprotectant in the aqueous composition (and, as a consequence, the solid composition) or the reconstituted aqueous composition is from about 1:1 (w/w) to about 1:50 (w/w). In an embodiment, the ratio of CMC or a pharmaceutically acceptable salt thereof to cryoprotectant in the aqueous composition (and, as a consequence, the solid composition) or the reconstituted aqueous composition is from about 1:1 (w/w) to about 1:40 (w/w). In a preferred embodiment, the ratio of CMC or a pharmaceutically acceptable salt thereof to cryoprotectant in the aqueous composition (and, as a consequence, the solid composition) or the reconstituted aqueous composition is from about 1:8 (w/w) to about 1:40 (w/w). In an embodiment, the ratio of CMC or a pharmaceutically acceptable salt thereof to cryoprotectant in the aqueous composition (and, as a consequence, the solid composition) or the reconstituted aqueous composition is from about 1:8 (w/w) to about 1:35 (w/w), e.g. about 1:10 (w/w) to about 1:33 (w/w).

In an embodiment, the cryoprotectant is arginine, e.g. arginine HCl, and the ratio of CMC or a pharmaceutically acceptable salt thereof to cryoprotectant in the aqueous composition (and, as a consequence, the solid composition) or the reconstituted aqueous composition is from about 1:1 (w/w) to about 1:30 (w/w). In an embodiment, the cryoprotectant is arginine, e.g. arginine HCl, and the ratio of CMC or a pharmaceutically acceptable salt thereof to cryoprotectant in the aqueous composition (and, as a consequence, the solid composition) or the reconstituted aqueous composition is from about 1:1 (w/w) to about 1:15 (w/w). In an embodiment, the cryoprotectant is arginine, e.g. arginine HCl, and the ratio of CMC or a pharmaceutically acceptable salt thereof to cryoprotectant in the aqueous composition (and, as a consequence, the solid composition) or the reconstituted aqueous composition is from about 1:2 (w/w) to about 1:15 (w/w). In an embodiment, the cryoprotectant is arginine, e.g. arginine HCl, and the ratio of CMC or a pharmaceutically acceptable salt thereof to cryoprotectant in the aqueous composition (and, as a consequence, the solid composition) or the reconstituted aqueous composition is from about 1:4 (w/w) to about 1:11 (w/w). In a preferred embodiment, the cryoprotectant is arginine, e.g. arginine HCl, and the ratio of CMC or a pharmaceutically acceptable salt thereof to cryoprotectant in the aqueous composition (and, as a consequence, the solid composition) or the reconstituted aqueous composition is from about 1:9 (w/w) to about 1:11 (w/w). In another preferred embodiment, the cryoprotectant is arginine, e.g. arginine HCl, and the ratio of CMC or a pharmaceutically acceptable salt thereof to cryoprotectant in the aqueous composition (and, as a consequence, the solid composition) or the reconstituted aqueous composition is from about 1:5 (w/w) to about 1:10 (w/w), e.g. about 1:10 (w/w).

In an embodiment, the cryoprotectant is sucrose, and the ratio of CMC or a pharmaceutically acceptable salt thereof to cryoprotectant in the aqueous composition (and, as a consequence, the solid composition) or the reconstituted aqueous composition is from about 1:20 (w/w) to about 1:100 (w/w). In an embodiment, the cryoprotectant is sucrose, and the ratio of CMC or a pharmaceutically acceptable salt thereof to cryoprotectant in the aqueous composition (and, as a consequence, the solid composition) or the reconstituted aqueous composition is from about 1:20 (w/w) to about 1:50 (w/w). In an embodiment, the cryoprotectant is sucrose, and the ratio of CMC or a pharmaceutically acceptable salt thereof to cryoprotectant in the aqueous composition (and, as a consequence, the solid composition) or the reconstituted aqueous composition is from about 1:30 (w/w) to about 1:40 (w/w). In a preferred embodiment, the cryoprotectant is sucrose, and the ratio of CMC or a pharmaceutically acceptable salt thereof to cryoprotectant in the aqueous composition (and, as a consequence, the solid composition) or the reconstituted aqueous composition is from about 1:30 (w/w) to about 1:35 (w/w), e.g. about 1:33 (w/w).

In an embodiment, the aqueous composition (and, as a consequence, the solid composition) or the reconstituted aqueous composition additionally comprises one or more surface modifiers. When the rilpivirine or a pharmaceutically acceptable salt thereof is in the form of particles as defined herein, the one or more surface modifiers is adsorbed to the surface of the particles.

The surface modifier may be selected from known organic and inorganic pharmaceutical excipients, including various polymers, low molecular weight oligomers, natural products and surfactants. Particular surface modifiers that may be used in the invention include nonionic and anionic surfactants. Representative examples of surface modifiers include gelatin, casein, lecithin, salts of negatively charged phospholipids or the acid form thereof (such as phosphatidyl glycerol, phosphatidyl inosite, phosphatidyl serine, phosphatic acid, and their salts such as alkali metal salts, e.g. their sodium salts, for example egg phosphatidyl glycerol sodium, such as the product available under the tradename Lipoid™ EPG), gum acacia, stearic acid, benzalkonium chloride, polyoxyethylene alkyl ethers, e.g., macrogol ethers such as cetomacrogol 1000, polyoxyethylene castor oil derivatives; polyoxyethylene stearates, colloidal silicon dioxide, sodium dodecylsulfate, bile salts such as sodium taurocholate, sodium desoxytaurocholate, sodium desoxycholate; methylcellulose, hydroxyethylcellulose, hydroxypropylcellulose, hydroxypropylmethyl-cellulose, magnesium aluminate silicate, polyvinyl alcohol (PVA), poloxamers, such as Pluronic™ F68, F108 and F127 which are block copolymers of ethylene oxide and propylene oxide; tyloxapol; Vitamin E-TGPS (α-tocopheryl polyethylene glycol succinate, in particular α-tocopheryl polyethylene glycol 1000 succinate); poloxamines, such as Tetronic™ 908 (T908) which is a tetrafunctional block copolymer derived from sequential addition of ethylene oxide and propylene oxide to ethylenediamine; dextran; lecithin; dioctyl ester of sodium sulfosuccinic acid such as the products sold under the tradename Aerosol OT™ (AOT); sodium lauryl sulfate (Duponol™ P); alkyl aryl polyether sulfonate available under the tradename Triton™ X-200; polyoxyethylene sorbitan fatty acid esters (Tweens™ 20, 40, 60 and 80); sorbitan esters of fatty acids (Span™ 20, 40, 60 and 80 or Arlacel™ 20, 40, 60 and 80); polyethylene glycols (such as those sold under the tradename Carbowax™ 3550 and 934); sucrose stearate and sucrose distearate mixtures such as the product available under the tradename Crodesta™ F110 or Crodesta™ SL-40; hexyldecyl trimethyl ammonium chloride (CTAC); polyvinylpyrrolidone (PVP). If desired, two or more surface modifiers can be used in combination.

In an embodiment, the surface modifier is selected from a poloxamer, α-tocopheryl polyethylene glycol succinate, polyoxyethylene sorbitan fatty acid ester, and salts of negatively charged phospholipids or the acid form thereof. In an embodiment, the surface modifier is selected from Pluronic™ F108, Vitamin E TGPS (α-tocopheryl polyethylene glycol succinate, in particular a-tocopheryl polyethylene glycol 1000 succinate), polyoxyethylene sorbitan fatty acid esters such as Tween™ 80, and phosphatidyl glycerol, phosphatidyl inosite, phosphatidyl serine, phosphatic acid, and their salts such as alkali metal salts, e.g. their sodium salts, for example egg phosphatidyl glycerol sodium, such as the product available under the tradename Lipoid™ EPG.

In a preferred embodiment, the surface modifier is a poloxamer, in particular Pluronic™ F108. Pluronic™ F108 corresponds to poloxamer 338 and is the polyoxyethylene, polyoxypropylene block copolymer that conforms generally to the formula HO—[CH₂CH₂O]ₓ—[CH(CH₃)CH₂O]_{y}—[CH₂CH₂O]ᵣ—H in which the average values of x, y and z are respectively 128, 54 and 128. Other commercial names of poloxamer 338 are Hodag Nonionic™ 1108-F and Synperonic™ PE/F108. In one embodiment, the surface modifier comprises a combination of a polyoxyethylene sorbitan fatty acid ester and a phosphatidyl glycerol salt (in particular egg phosphatidyl glycerol sodium).

In an embodiment, the aqueous composition (and, as a consequence, the solid composition) or the reconstituted aqueous composition comprises from about 0.1 mg/mL to about 100 mg/mL of a poloxamer. In an embodiment, the aqueous composition (and, as a consequence, the solid composition) or the reconstituted aqueous composition comprises from about 1 mg/mL to about 100 mg/mL of a poloxamer. In an embodiment, the aqueous composition (and, as a consequence, the solid composition) or the reconstituted aqueous composition comprises from about 5 mg/mL to about 100 mg/mL of a poloxamer. In an embodiment, the aqueous composition (and, as a consequence, the solid composition) or the reconstituted aqueous composition comprises from about 5 mg/mL to about 70 mg/mL of a poloxamer. In an embodiment, the aqueous composition (and, as a consequence, the solid composition) or the reconstituted aqueous composition comprises from about 5 mg/mL to about 60 mg/mL of a poloxamer.

In an embodiment, the aqueous composition (and, as a consequence, the solid composition) or the reconstituted aqueous composition comprises from about 5 mg/mL to about 25 mg/mL of a poloxamer. In an embodiment, the aqueous composition (and, as a consequence, the solid composition) or the reconstituted aqueous composition comprises from about 5 mg/mL to about 20 mg/mL of a poloxamer, e.g. about 20 mg/mL. In a preferred embodiment, the aqueous composition (and, as a consequence, the solid composition) or the reconstituted aqueous composition comprises from about 5 mg/mL to about 15 mg/mL of a poloxamer, e.g. about 10 mg/mL.

In an embodiment, the aqueous composition (and, as a consequence, the solid composition) or the reconstituted aqueous composition comprises from about 20 mg/mL to about 60 mg/mL of a poloxamer. In an embodiment, the aqueous composition (and, as a consequence, the solid composition) or the reconstituted aqueous composition comprises from about 25 mg/mL to about 60 mg/mL of a poloxamer. In a particularly preferred embodiment, the aqueous composition (and, as a consequence, the solid composition) or the reconstituted aqueous composition comprises from about 40 mg/mL to about 60 mg/mL of a poloxamer, e.g. about 50 mg/mL.

In an embodiment, the aqueous composition (and, as a consequence, the solid composition) or the reconstituted aqueous composition comprises a poloxamer and rilpivirine or a pharmaceutically acceptable salt thereof in the form of particles suspended in the aqueous composition or the reconstituted aqueous composition, wherein when the rilpivirine or a pharmaceutically acceptable salt thereof has a Dv90 of less than about 1600 nm the ratio of rilpivirine or a pharmaceutically acceptable salt thereof to poloxamer is less than about 15:1 (w/w). In an embodiment, the aqueous composition (and, as a consequence, the solid composition) or the reconstituted aqueous composition comprises a poloxamer and rilpivirine or a pharmaceutically acceptable salt thereof in the form of particles suspended in the aqueous composition or the reconstituted aqueous composition, wherein when the rilpivirine or a pharmaceutically acceptable salt thereof has a Dv90 of less than about 1600 nm the ratio of rilpivirine or a pharmaceutically acceptable salt thereof to poloxamer is from about 15:1 (w/w) to about 3:1 (w/w). In an embodiment, the aqueous composition (and, as a consequence, the solid composition) or the reconstituted aqueous composition comprises a poloxamer and rilpivirine or a pharmaceutically acceptable salt thereof in the form of particles suspended in the aqueous composition or the reconstituted aqueous composition, wherein when the rilpivirine or a pharmaceutically acceptable salt thereof has a Dv90 of less than about 1600 nm the ratio of rilpivirine or a pharmaceutically acceptable salt thereof to poloxamer is from about 15:1 (w/w) to about 4:1 (w/w). In an embodiment, the aqueous composition (and, as a consequence, the solid composition) or the reconstituted aqueous composition comprises a poloxamer and rilpivirine or a pharmaceutically acceptable salt thereof in the form of particles suspended in the aqueous composition or the reconstituted aqueous composition, wherein when the rilpivirine or a pharmaceutically acceptable salt thereof has a Dv90 of less than about 1600 nm the ratio of rilpivirine or a pharmaceutically acceptable salt thereof to poloxamer is from about 15:1 (w/w) to about 6:1 (w/w). In a preferred embodiment, the aqueous composition (and, as a consequence, the solid composition) or the reconstituted aqueous composition comprises a poloxamer and rilpivirine or a pharmaceutically acceptable salt thereof in the form of particles suspended in the aqueous composition or the reconstituted aqueous composition, wherein when the rilpivirine or a pharmaceutically acceptable salt thereof has a Dv90 of less than about 1600 nm the ratio of rilpivirine or a pharmaceutically acceptable salt thereof to poloxamer is about 6:1 (w/w).

In an embodiment, the aqueous composition (and, as a consequence, the solid composition) or the reconstituted aqueous composition comprises rilpivirine or a pharmaceutically acceptable salt thereof in the form of particles suspended in the aqueous composition or the reconstituted aqueous composition and a poloxamer, the ratio of the rilpivirine or a pharmaceutically acceptable salt thereof to poloxamer is more than about 15:1 (w/w), and the rilpivirine or a pharmaceutically acceptable salt thereof has a Dv90 of from about 1 μm to about 10 μm, from about 2 μm to about 9 μm, from about 3 μm to about 8 μm, or from about 3 μm to about 7 μm. In an embodiment, the aqueous composition (and, as a consequence, the solid composition) or the reconstituted aqueous composition comprises rilpivirine or a pharmaceutically acceptable salt thereof in the form of particles suspended in the aqueous composition or the reconstituted aqueous composition, and a poloxamer, the ratio of the rilpivirine or a pharmaceutically acceptable salt thereof to poloxamer is from about 15:1 (w/w) to about 60:1 (w/w), and the rilpivirine or a pharmaceutically acceptable salt thereof has a Dv90 of from about 1 μm to about 10 μm, from about 2 μm to about 9 μm, from about 3 μm to about 8 μm, or from about 3 μm to about 7 μm. In an embodiment, the aqueous composition (and, as a consequence, the solid composition) or the reconstituted aqueous composition comprises rilpivirine or a pharmaceutically acceptable salt thereof in the form of particles suspended in the aqueous composition or the reconstituted aqueous composition, and a poloxamer, the ratio of the rilpivirine or a pharmaceutically acceptable salt thereof to poloxamer is from about 20:1 (w/w) to about 60:1 (w/w), and the rilpivirine or a pharmaceutically acceptable salt thereof has a Dv90 of from about 1 μm to about 10 μm, from about 2 μm to about 9 μm, from about 3 μm to about 8 μm, or from about 3 μm to about 7 μm. In an embodiment, the aqueous composition (and, as a consequence, the solid composition) or the reconstituted aqueous composition comprises rilpivirine or a pharmaceutically acceptable salt thereof in the form of particles suspended in the aqueous composition or the reconstituted aqueous composition, and a poloxamer, the ratio of the rilpivirine or a pharmaceutically acceptable salt thereof to poloxamer is from about 30:1 (w/w) to about 60:1 (w/w), and the rilpivirine or a pharmaceutically acceptable salt thereof has a Dv90 of from about 1 μm to about 10 μm, from about 2 μm to about 9 μm, from about 3 μm to about 8 μm, or from about 3 μm to about 7 μm. In a preferred embodiment, the aqueous composition (and, as a consequence, the solid composition) or the reconstituted aqueous composition comprises rilpivirine or a pharmaceutically acceptable salt thereof in the form of particles suspended in the aqueous composition or the reconstituted aqueous composition, and a poloxamer, the ratio of the rilpivirine or a pharmaceutically acceptable salt thereof to poloxamer is about 30:1 (w/w), and the rilpivirine or a pharmaceutically acceptable salt thereof has a Dv90 of from about 1 μm to about 10 μm, from about 2 μm to about 9 μm, from about 3 μm to about 8 μm, or from about 3 μm to about 7 μm, e.g. from about 4 μm to about 6 μm.

In an embodiment, the aqueous composition (and, as a consequence, the solid composition) or the reconstituted aqueous composition comprises rilpivirine or a pharmaceutically acceptable salt thereof in the form of particles suspended in the aqueous composition or the reconstituted aqueous composition, and a poloxamer, the ratio of the rilpivirine or a pharmaceutically acceptable salt thereof to poloxamer is less than about 15:1 (w/w), and the rilpivirine or a pharmaceutically acceptable salt thereof has a Dv90 of from about 500 nm to about 1600 nm. In an embodiment, the aqueous composition (and, as a consequence, the solid composition) or the reconstituted aqueous composition comprises rilpivirine or a pharmaceutically acceptable salt thereof in the form of particles suspended in the aqueous composition or the reconstituted aqueous composition, and a poloxamer, the ratio of the rilpivirine or a pharmaceutically acceptable salt thereof to poloxamer is from about 3:1 (w/w) to about 15:1 (w/w), and the rilpivirine or a pharmaceutically acceptable salt thereof has a Dv90 of from about 500 nm to about 1600 nm. In an embodiment, the aqueous composition (and, as a consequence, the solid composition) or the reconstituted aqueous composition comprises rilpivirine or a pharmaceutically acceptable salt thereof in the form of particles suspended in the aqueous composition or the reconstituted aqueous composition, and a poloxamer, the ratio of the rilpivirine or a pharmaceutically acceptable salt thereof to poloxamer is from about 4:1 (w/w) to about 15:1 (w/w), and the rilpivirine or a pharmaceutically acceptable salt thereof has a Dv90 of from about 500 nm to about 1600 nm. In an embodiment, the aqueous composition (and, as a consequence, the solid composition) or the reconstituted aqueous composition comprises rilpivirine or a pharmaceutically acceptable salt thereof in the form of particles suspended in the aqueous composition or the reconstituted aqueous composition, and a poloxamer, the ratio of the rilpivirine or a pharmaceutically acceptable salt thereof to poloxamer is from about 6:1 (w/w) to about 15:1 (w/w), and the rilpivirine or a pharmaceutically acceptable salt thereof has a Dv90 of from about 500 nm to about 1600 nm. In a preferred embodiment, the aqueous composition (and, as a consequence, the solid composition) or the reconstituted aqueous composition comprises rilpivirine or a pharmaceutically acceptable salt thereof in the form of particles suspended in the aqueous composition or the reconstituted aqueous composition, and a poloxamer, the ratio of the rilpivirine or a pharmaceutically acceptable salt thereof to poloxamer is about 6:1 (w/w), and the rilpivirine or a pharmaceutically acceptable salt thereof has a Dv90 of from about 500 nm to about 1600 nm, e.g. about 500 nm to about 700 nm.

In an embodiment, the relative amount (w/w) of rilpivirine or a pharmaceutically acceptable salt thereof to the surface modifier is from about 1:1 to about 40:1. In an embodiment, the relative amount (w/w) of rilpivirine or a pharmaceutically acceptable salt thereof to the surface modifier is from about 1:1 to about 35:1. In an embodiment, the relative amount (w/w) of rilpivirine or a pharmaceutically acceptable salt thereof to the surface modifier is from about 15:1 to about 35:1. In an embodiment, the relative amount (w/w) of rilpivirine or a pharmaceutically acceptable salt thereof to the surface modifier is from about 25:1 to about 35:1, e.g. about 30:1. The surface modifier is preferably a poloxamer, e.g. poloxamer 338.

In an embodiment, the relative amount (w/w) of rilpivirine or a pharmaceutically acceptable salt thereof to the surface modifier is from about 5:1 to about 25:1. In an embodiment, the relative amount (w/w) of rilpivirine or a pharmaceutically acceptable salt thereof to the surface modifier is from about 10:1 to about 20:1. In an embodiment, the relative amount (w/w) of rilpivirine or a pharmaceutically acceptable salt thereof to the surface modifier is from about 13:1 to about 17:1, e.g. about 15:1. The surface modifier is preferably a poloxamer, e.g. poloxamer 338.

In an embodiment, the relative amount (w/w) of rilpivirine or a pharmaceutically acceptable salt thereof to the surface modifier (e.g. a poloxamer) is less than about 15:1. In a preferred embodiment, the relative amount (w/w) of rilpivirine or a pharmaceutically acceptable salt thereof to the surface modifier (e.g. a poloxamer such as poloxamer 338) is less than about 12:1.

In an embodiment, the relative amount (w/w) of rilpivirine or a pharmaceutically acceptable salt thereof to the surface modifier is from about 1:2 to about 20:1, in particular from about 1:1 to about 10:1, such as from about 4:1 to about 8:1, e.g. from about 4:1 to about 6:1, preferably about 6:1. The surface modifier is preferably a poloxamer, e.g. poloxamer 338.

In an embodiment, the aqueous composition or the reconstituted aqueous composition comprises rilpivirine or a pharmaceutically acceptable salt thereof as defined herein in the form of particles suspended in the aqueous composition or the reconstituted aqueous composition and one or more surface modifiers as defined herein wherein the amount of rilpivirine or a pharmaceutically acceptable salt thereof is at least about 50% by weight of the particles, at least about 80% by weight of the particles, at least about 85% by weight of the particles, at least about 90% by weight of the particles, at least about 95% by weight of the particles, or at least about 99% by weight of the particles, in particular ranges between 80% and 90% by weight of the particles or ranges between 85% and 90% by weight of the particles.

The aqueous composition or the reconstituted aqueous composition comprises water, e.g. sterile water for injection.

In an embodiment, the aqueous composition (and, as a consequence, the solid composition) or the reconstituted aqueous composition additionally comprises a buffering agent and/or a pH adjusting agent. Particular buffers are the salts of weak acids. Buffering and pH adjusting agents that can be added may be selected from tartaric acid, maleic acid, glycine, sodium lactate/lactic acid, ascorbic acid, sodium citrates/citric acid, sodium acetate/acetic acid, sodium bicarbonate/carbonic acid, sodium succinate/succinic acid, sodium benzoate/benzoic acid, sodium phosphates, tris(hydroxymethyl)aminomethane, sodium bicarbonate/sodium carbonate, ammonium hydroxide, benzene sulfonic acid, benzoate sodium/acid, diethanolamine, glucono delta lactone, hydrochloric acid, hydrogen bromide, lysine, methanesulfonic acid, monoethanolamine, sodium hydroxide, tromethamine, gluconic, glyceric, gluratic, glutamic, ethylene diamine tetraacetic (EDTA), triethanolamine, including mixtures thereof. In an embodiment, the buffer is a sodium phosphate buffer, e.g. sodium dihydrogen phosphate monohydrate. In an embodiment the pH adjusting agent is sodium hydroxide. In a preferred embodiment, the aqueous composition or the reconstituted aqueous composition comprises a buffering agent and a pH adjusting agent, wherein the buffering agent is sodium dihydrogen phosphate monohydrate and the pH adjusting agent is sodium hydroxide.

In an embodiment, the pH of the aqueous composition or the reconstituted aqueous composition is from about 5 to about 7. In an embodiment, the pH of the aqueous composition or the reconstituted aqueous composition is from about 6 to about 7. In an embodiment, the pH of the aqueous composition or the reconstituted aqueous composition is from about 6 to about 6.5. In a preferred embodiment, the pH of the aqueous composition or the reconstituted aqueous composition is about 6. The pH of the aqueous composition or the reconstituted aqueous composition may be adjusted by, for example, varying the type and/or amount of buffering agent and/or pH adjusting agent present in the aqueous composition.

In an embodiment, the aqueous composition (and, as a consequence, the solid composition) or the reconstituted aqueous composition additionally comprises a chelating agent. In an embodiment the chelating agent is selected form sodium citrate, sodium EDTA, citric acid and malic acid. In a preferred embodiment, the chelating agent is citric acid, e.g. citric acid monohydrate.

In an embodiment, the aqueous composition (and, as a consequence, the solid composition) or the reconstituted aqueous composition additionally comprises an antioxidant. In an embodiment, the antioxidant is methionine. In an embodiment, the aqueous composition (and, as a consequence, the solid composition) or the reconstituted aqueous composition comprises from about 1.0 mg/mL to about 2.0 mg/mL of the antioxidant (e.g. methionine), for example about 1.5 mg/mL.

In an embodiment, the aqueous composition (and, as a consequence, the solid composition) or the reconstituted aqueous composition does not comprise an isotonizing agent.

In an embodiment, the aqueous composition (and, as a consequence, the solid composition) or the reconstituted aqueous composition does not comprise glucose.

In an embodiment, the aqueous composition (and, as a consequence, the solid composition) or the reconstituted aqueous composition does not comprise mannitol.

In an embodiment, the aqueous composition (and, as a consequence, the solid composition) or the reconstituted aqueous composition does not comprise a polyvinyl pyrrolidone (PVP).

The aqueous composition and the reconstituted aqueous composition may usefully have osmolality suitable to reduce pain on subcutaneous injection. For example, in an embodiment, the aqueous composition and reconstituted aqueous composition has an osmolality of from about 280 mOsm/kg to about 600 mOsm/kg. In an embodiment, the aqueous composition and reconstituted aqueous composition has an osmolality of from about 280 mOsm/kg to about 300 mOsm/kg. In a preferred embodiment, the aqueous composition and reconstituted aqueous composition has an osmolality of about 290 mOsm/kg.

In an embodiment, the aqueous composition or the reconstituted aqueous composition of the invention may be housed in a container with an inert gas. The inert gas may, for example, be nitrogen.

The following particular embodiments are also part of the invention.

In a particular embodiment, the aqueous composition or the reconstituted aqueous composition comprises:
a hyaluronidase;
rilpivirine or a pharmaceutically acceptable salt thereof; and CMC or a pharmaceutically acceptable salt thereof,
wherein the pH of the aqueous composition or the reconstituted aqueous composition is from about 6 to about 7,
optionally wherein the aqueous composition or the reconstituted aqueous composition additionally comprises one or more component selected from a surfactant, e.g. a poloxamer, a buffering agent, e.g. sodium dihydrogen phosphate, a chelating agent, e.g. citric acid, and a pH adjusting agent, e.g. sodium hydroxide, further optionally wherein the aqueous composition or the reconstituted aqueous composition additionally comprises poloxamer, sodium dihydrogen phosphate, citric acid, and sodium hydroxide.

In a particular embodiment, the aqueous composition or the reconstituted aqueous composition comprises:
rHuPH20;
rilpivirine; and
sodium CMC,
wherein the pH of the aqueous composition or the reconstituted aqueous composition is from about 6 to about 6.5,
optionally wherein the aqueous composition or the reconstituted aqueous composition additionally comprises one or more component selected from poloxamer 338, sodium dihydrogen phosphate monohydrate, citric acid monohydrate, and sodium hydroxide, further optionally wherein the aqueous composition or the reconstituted aqueous composition additionally comprises poloxamer 338, sodium dihydrogen phosphate monohydrate, citric acid monohydrate, and sodium hydroxide.

In a particular embodiment, the aqueous composition or the reconstituted aqueous composition comprises:
rHuPH20;
rilpivirine; and
sodium CMC,
wherein the pH of the aqueous composition or the reconstituted aqueous composition is from about 6 to about 6.5, and
wherein the aqueous composition comprises from about 0.5 mg sodium CMC per 100 U rHuPH20 to about 2 mg sodium CMC per 100 U rHuPH20,
optionally wherein the aqueous composition or the reconstituted aqueous composition additionally comprises one or more component selected from poloxamer 338, sodium dihydrogen phosphate monohydrate, citric acid monohydrate, and sodium hydroxide, further optionally wherein the aqueous composition or the reconstituted aqueous composition additionally comprises poloxamer 338, sodium dihydrogen phosphate monohydrate, citric acid monohydrate, and sodium hydroxide.

In a particular embodiment, the aqueous composition or the reconstituted aqueous composition comprises:
rHuPH2O;
rilpivirine; and
sodium CMC,
wherein the pH of the aqueous composition or the reconstituted aqueous composition is from about 6 to about 6.5, and
wherein the aqueous composition comprises from about 0.05 mg sodium CMC per 100 U rHuPH20 to about 0.25 mg sodium CMC per 100 U rHuPH20, optionally wherein the aqueous composition or the reconstituted aqueous composition additionally comprises one or more component selected from poloxamer 338, sodium dihydrogen phosphate monohydrate, citric acid monohydrate, and sodium hydroxide, further optionally wherein the aqueous composition or the reconstituted aqueous composition additionally comprises poloxamer 338, sodium dihydrogen phosphate monohydrate, citric acid monohydrate, and sodium hydroxide.

In a particular embodiment, the aqueous composition or the reconstituted aqueous composition comprises:
a hyaluronidase;
rilpivirine or a pharmaceutically acceptable salt thereof;
CMC or a pharmaceutically acceptable salt thereof; and
a cryoprotectant,
wherein the pH of the aqueous composition or the reconstituted aqueous composition is from about 6 to about 7,
optionally wherein the aqueous composition or the reconstituted aqueous composition additionally comprises one or more component selected from a surfactant, e.g. a poloxamer, a buffering agent, e.g. sodium dihydrogen phosphate, a chelating agent, e.g. citric acid, and a pH adjusting agent, e.g. sodium hydroxide, further optionally wherein the aqueous composition or the reconstituted aqueous composition additionally comprises poloxamer, sodium dihydrogen phosphate, citric acid, and sodium hydroxide.

In a particular embodiment, the aqueous composition or the reconstituted aqueous composition comprises:
rHuPH2O;
rilpivirine;
sodium CMC; and
a sugar or sugar alcohol,
wherein the pH of the aqueous composition or the reconstituted aqueous composition is from about 6 to about 6.5,
optionally wherein the aqueous composition or the reconstituted aqueous composition additionally comprises one or more component selected from poloxamer 338, sodium dihydrogen phosphate monohydrate, citric acid monohydrate, and sodium hydroxide, further optionally wherein the aqueous composition or the reconstituted aqueous composition additionally comprises poloxamer 338, sodium dihydrogen phosphate monohydrate, citric acid monohydrate, and sodium hydroxide.

In a particular embodiment, the aqueous composition or the reconstituted aqueous composition comprises:
rHuPH2O;
rilpivirine;
sodium CMC; and
mannitol and/or sucrose,
wherein the pH of the aqueous composition or the reconstituted aqueous composition is from about 6 to about 6.5,
optionally wherein the aqueous composition or the reconstituted aqueous composition additionally comprises one or more component selected from poloxamer 338, sodium dihydrogen phosphate monohydrate, citric acid monohydrate, and sodium hydroxide, further optionally wherein the aqueous composition or the reconstituted aqueous composition additionally comprises poloxamer 338, sodium dihydrogen phosphate monohydrate, citric acid monohydrate, and sodium hydroxide.

In a particular embodiment, the aqueous composition or the reconstituted aqueous composition comprises:
rHuPH2O;
rilpivirine;
sodium CMC; and
sucrose,
wherein the pH of the aqueous composition or the reconstituted aqueous composition is from about 6 to about 6.5,
optionally wherein the aqueous composition or the reconstituted aqueous composition additionally comprises one or more component selected from poloxamer 338, sodium dihydrogen phosphate monohydrate, citric acid monohydrate, and sodium hydroxide, further optionally wherein the aqueous composition or the reconstituted aqueous composition additionally comprises poloxamer 338, sodium dihydrogen phosphate monohydrate, citric acid monohydrate, and sodium hydroxide.

In a particular embodiment, the aqueous composition or the reconstituted aqueous composition comprises:
rHuPH2O;
rilpivirine;
sodium CMC; and
sucrose,
wherein the pH of the aqueous composition or the reconstituted aqueous composition is from about 6 to about 6.5,
wherein the aqueous composition or the reconstituted aqueous composition comprises from about 0.05 mg sodium CMC per 100 U rHuPH20 to about 0.25 mg sodium CMC per 100 U rHuPH20,
optionally wherein the aqueous composition or the reconstituted aqueous composition additionally comprises one or more component selected from poloxamer 338, sodium dihydrogen phosphate monohydrate, citric acid monohydrate, and sodium hydroxide, further optionally wherein the aqueous composition or the reconstituted aqueous composition additionally comprises poloxamer 338, sodium dihydrogen phosphate monohydrate, citric acid monohydrate, and sodium hydroxide.

In a particular embodiment, the aqueous composition or the reconstituted aqueous composition comprises:
rHuPH20;
rilpivirine;
sodium CMC; and
mannitol and/or sucrose,
wherein the pH of the aqueous composition or the reconstituted aqueous composition is from about 6 to about 6.5,
wherein the aqueous composition or the reconstituted aqueous composition comprises from about 0.5 mg sodium CMC per 100 U rHuPH20 to about 2 mg sodium CMC per 100 U rHuPH2O,
wherein the ratio of sodium CMC to mannitol or sucrose is from about 1:4 (w/w) to about 1:11 (w/w), and
wherein the ratio of rilpivirine to mannitol or sucrose is from about 2:1 (w/w) to about 7:1 (w/w),
optionally wherein the aqueous composition or the reconstituted aqueous composition additionally comprises one or more component selected from poloxamer 338, sodium dihydrogen phosphate monohydrate, citric acid monohydrate, and sodium hydroxide, further optionally wherein the aqueous composition or the reconstituted aqueous composition additionally comprises poloxamer 338, sodium dihydrogen phosphate monohydrate, citric acid monohydrate, and sodium hydroxide.

In a particular embodiment, the aqueous composition or the reconstituted aqueous composition comprises:
rHuPH20;
rilpivirine;
sodium CMC; and
sucrose,
wherein the pH of the aqueous composition or the reconstituted aqueous composition is from about 6 to about 6.5,
wherein the aqueous composition or the reconstituted aqueous composition comprises from about 0.05 mg sodium CMC per 100 U rHuPH20 to about 0.25 mg sodium CMC per 100 U rHuPH20,
wherein the ratio of sodium CMC to sucrose is from about 1:30 (w/w) to about 1:35 (w/w), and
wherein the ratio of rilpivirine to sucrose is from about 2:1 (w/w) to about 7:1 (w/w), optionally wherein the aqueous composition or the reconstituted aqueous composition additionally comprises one or more component selected from poloxamer 338, sodium dihydrogen phosphate monohydrate, citric acid monohydrate, and sodium hydroxide, further optionally wherein the aqueous composition or the reconstituted aqueous composition additionally comprises poloxamer 338, sodium dihydrogen phosphate monohydrate, citric acid monohydrate, and sodium hydroxide.

In a particular embodiment, the aqueous composition or the reconstituted aqueous composition comprises:
a hyaluronidase;
rilpivirine or a pharmaceutically acceptable salt thereof; and
a cryoprotectant,
wherein the pH of the aqueous composition or the reconstituted aqueous composition is from about 6 to about 7,
optionally wherein the aqueous composition or the reconstituted aqueous composition additionally comprises one or more component selected from a surfactant, e.g. a poloxamer, a buffering agent, e.g. sodium dihydrogen phosphate, a chelating agent, e.g. citric acid, and a pH adjusting agent, e.g. sodium hydroxide, further optionally wherein the aqueous composition or the reconstituted aqueous composition additionally comprises poloxamer, sodium dihydrogen phosphate, citric acid, and sodium hydroxide.

In a particular embodiment, the aqueous composition or the reconstituted aqueous composition comprises:
rHuPH2O;
rilpivirine; and
an amino acid or a pharmaceutically acceptable salt thereof,
wherein the pH of the aqueous composition or the reconstituted aqueous composition is from about 6 to about 6.5,
optionally wherein the aqueous composition or the reconstituted aqueous composition additionally comprises one or more component selected from poloxamer 338, sodium dihydrogen phosphate monohydrate, citric acid monohydrate, and sodium hydroxide, further optionally wherein the aqueous composition or the reconstituted aqueous composition additionally comprises poloxamer 338, sodium dihydrogen phosphate monohydrate, citric acid monohydrate, and sodium hydroxide.

In a particular embodiment, the aqueous composition or the reconstituted aqueous composition comprises:
rHuPH2O;
rilpivirine; and
arginine HCl,
wherein the pH of the aqueous composition or the reconstituted aqueous composition is from about 6 to about 6.5,
optionally wherein the aqueous composition or the reconstituted aqueous composition additionally comprises one or more component selected from poloxamer 338, sodium dihydrogen phosphate monohydrate, citric acid monohydrate, and sodium hydroxide, further optionally wherein the aqueous composition or the reconstituted aqueous composition additionally comprises poloxamer 338, sodium dihydrogen phosphate monohydrate, citric acid monohydrate, and sodium hydroxide.

In a particular embodiment, the aqueous composition or the reconstituted aqueous composition comprises:
rHuPH2O;
rilpivirine;
CMC or a pharmaceutically acceptable salt thereof; and
arginine HCl,
wherein the pH of the aqueous composition or the reconstituted aqueous composition is from about 6 to about 6.5,
optionally wherein the aqueous composition or the reconstituted aqueous composition additionally comprises one or more component selected from poloxamer 338, sodium dihydrogen phosphate monohydrate, citric acid monohydrate, and sodium hydroxide, further optionally wherein the aqueous composition or the reconstituted aqueous composition additionally comprises poloxamer 338, sodium dihydrogen phosphate monohydrate, citric acid monohydrate, and sodium hydroxide.

In a particular embodiment, the aqueous composition or the reconstituted aqueous composition comprises:
rHuPH2O;
rilpivirine;
sodium CMC; and
arginine HCl,
wherein the pH of the aqueous composition or the reconstituted aqueous composition is from about 6 to about 6.5,
optionally wherein the aqueous composition or the reconstituted aqueous composition additionally comprises one or more component selected from poloxamer 338, sodium dihydrogen phosphate monohydrate, citric acid monohydrate, and sodium hydroxide, further optionally wherein the aqueous composition or the reconstituted aqueous composition additionally comprises poloxamer 338, sodium dihydrogen phosphate monohydrate, citric acid monohydrate, and sodium hydroxide.

In a particular embodiment, the aqueous composition or the reconstituted aqueous composition comprises:
rHuPH2O;
rilpivirine; and
arginine HCl,
wherein the pH of the aqueous composition or the reconstituted aqueous composition is from about 6 to about 6.5, and
wherein the ratio of rilpivirine to arginine HCl is from about 1:1 (w/w) to about 20:1 (w/w), optionally wherein the aqueous composition or the reconstituted aqueous composition additionally comprises one or more component selected from poloxamer 338, sodium dihydrogen phosphate monohydrate, citric acid monohydrate, and sodium hydroxide, further optionally wherein the aqueous composition or the reconstituted aqueous composition additionally comprises poloxamer 338, sodium dihydrogen phosphate monohydrate, citric acid monohydrate, and sodium hydroxide.

In a particular embodiment, the aqueous composition or the reconstituted aqueous composition comprises:
rHuPH20;
rilpivirine; and
arginine HCl,
wherein the pH of the aqueous composition or the reconstituted aqueous composition is from about 6 to about 6.5, and
wherein the ratio of rilpivirine to arginine is from about 2:1 (w/w) to about 7:1 (w/w), optionally wherein the aqueous composition or the reconstituted aqueous composition additionally comprises one or more component selected from poloxamer 338, sodium dihydrogen phosphate monohydrate, citric acid monohydrate, and sodium hydroxide, further optionally wherein the aqueous composition or the reconstituted aqueous composition additionally comprises poloxamer 338, sodium dihydrogen phosphate monohydrate, citric acid monohydrate, and sodium hydroxide.

In a particular embodiment, the aqueous composition or the reconstituted aqueous composition comprises:
rHuPH20;
rilpivirine; and
arginine HCl,
wherein the pH of the aqueous composition or the reconstituted aqueous composition is from about 6 to about 6.5, and
wherein the ratio of rilpivirine to arginine HCl is from about 2:1 (w/w) to about 7:1 (w/w), optionally wherein the aqueous composition or the reconstituted aqueous composition additionally comprises one or more component selected from poloxamer 338, sodium dihydrogen phosphate monohydrate, citric acid monohydrate, and sodium hydroxide, further optionally wherein the aqueous composition or the reconstituted aqueous composition additionally comprises poloxamer 338, sodium dihydrogen phosphate monohydrate, citric acid monohydrate, and sodium hydroxide.

In a particular embodiment, the aqueous composition or the reconstituted aqueous composition comprises:
rHuPH20;
rilpivirine;
CMC or a pharmaceutically acceptable salt thereof; and
arginine HCl,
wherein the pH of the aqueous composition or the reconstituted aqueous composition is from about 6 to about 6.5, and
wherein the ratio of rilpivirine to arginine HCl is from about 1:1 (w/w) to about 20:1 (w/w), optionally wherein the aqueous composition or the reconstituted aqueous composition additionally comprises one or more component selected from poloxamer 338, sodium dihydrogen phosphate monohydrate, citric acid monohydrate, and sodium hydroxide, further optionally wherein the aqueous composition or the reconstituted aqueous composition additionally comprises poloxamer 338, sodium dihydrogen phosphate monohydrate, citric acid monohydrate, and sodium hydroxide.

In a particular embodiment, the aqueous composition or the reconstituted aqueous composition comprises:
rHuPH20;
rilpivirine;
CMC or a pharmaceutically acceptable salt thereof; and
arginine HCl,
wherein the pH of the aqueous composition or the reconstituted aqueous composition is from about 6 to about 6.5,
wherein the ratio of rilpivirine to arginine HCl is from about 1:1 (w/w) to about 20:1 (w/w),
wherein the ratio of CMC or a pharmaceutically acceptable salt thereof to arginine HCl is from about 1:5 (w/w) to about 1:10 (w/w).
optionally wherein the aqueous composition or the reconstituted aqueous composition additionally comprises one or more component selected from poloxamer 338, sodium dihydrogen phosphate monohydrate, citric acid monohydrate, and sodium hydroxide, further optionally wherein the aqueous composition or the reconstituted aqueous composition additionally comprises poloxamer 338, sodium dihydrogen phosphate monohydrate, citric acid monohydrate, and sodium hydroxide.

In a particular embodiment, the aqueous composition or the reconstituted aqueous composition comprises:
rHuPH20;
rilpivirine;
CMC or a pharmaceutically acceptable salt thereof; and
arginine HCl,
wherein the pH of the aqueous composition or the reconstituted aqueous composition is from about 6 to about 6.5,
wherein the aqueous composition or the reconstituted aqueous composition comprises from about 0.05 mg CMC or a pharmaceutically acceptable salt thereof per 100 U rHuPH20 to about 0.25 mg CMC or a pharmaceutically acceptable salt thereof per 100 U rHuPH20,
wherein the ratio of rilpivirine to arginine HCl is from about 1:1 (w/w) to about 20:1 (w/w),
wherein the ratio of CMC or a pharmaceutically acceptable salt thereof to arginine HCl is from about 1:5 (w/w) to about 1:10 (w/w).
optionally wherein the aqueous composition or the reconstituted aqueous composition additionally comprises one or more component selected from poloxamer 338, sodium dihydrogen phosphate monohydrate, citric acid monohydrate, and sodium hydroxide, further optionally wherein the aqueous composition or the reconstituted aqueous composition additionally comprises poloxamer 338, sodium dihydrogen phosphate monohydrate, citric acid monohydrate, and sodium hydroxide.

In a particular embodiment, the aqueous composition or the reconstituted aqueous composition comprises:
rHuPH20;
rilpivirine;
sodium CMC; and
arginine HCl,
wherein the pH of the aqueous composition or the reconstituted aqueous composition is from about 6 to about 6.5, and
wherein the ratio of rilpivirine to arginine HCl is from about 1:1 (w/w) to about 20:1 (w/w), optionally wherein the aqueous composition or the reconstituted aqueous composition additionally comprises one or more component selected from poloxamer 338, sodium dihydrogen phosphate monohydrate, citric acid monohydrate, and sodium hydroxide, further optionally wherein the aqueous composition or the reconstituted aqueous composition additionally comprises poloxamer 338, sodium dihydrogen phosphate monohydrate, citric acid monohydrate, and sodium hydroxide.

In a particular embodiment, the aqueous composition or the reconstituted aqueous composition comprises:
a hyaluronidase;
rilpivirine or a pharmaceutically acceptable salt thereof; and
two cryoprotectants,
wherein the pH of the aqueous composition or the reconstituted aqueous composition is from about 6 to about 7,
optionally wherein the aqueous composition or the reconstituted aqueous composition additionally comprises one or more component selected from a surfactant, e.g. a poloxamer, a buffering agent, e.g. sodium dihydrogen phosphate, a chelating agent, e.g. citric acid, and a pH adjusting agent, e.g. sodium hydroxide, further optionally wherein the aqueous composition or the reconstituted aqueous composition additionally comprises poloxamer, sodium dihydrogen phosphate, citric acid, and sodium hydroxide.

In a particular embodiment, the aqueous composition or the reconstituted aqueous composition comprises:
rHuPH20;
rilpivirine; and
a sugar or sugar alcohol and an amino acid or a pharmaceutically acceptable salt thereof, wherein the pH of the aqueous composition or the reconstituted aqueous composition is from about 6 to about 6.5,
optionally wherein the aqueous composition or the reconstituted aqueous composition additionally comprises one or more component selected from poloxamer 338, sodium dihydrogen phosphate monohydrate, citric acid monohydrate, and sodium hydroxide, further optionally wherein the aqueous composition or the reconstituted aqueous composition additionally comprises poloxamer 338, sodium dihydrogen phosphate monohydrate, citric acid monohydrate, and sodium hydroxide.

In a particular embodiment, the aqueous composition or the reconstituted aqueous composition comprises:
rHuPH20;
rilpivirine; and
a sugar or sugar alcohol and an amino acid or a pharmaceutically acceptable salt thereof, wherein the pH of the aqueous composition or the reconstituted aqueous composition is from about 6 to about 6.5, and
wherein the ratio of rilpivirine to the sugar or sugar alcohol and amino acid or a pharmaceutically acceptable salt thereof is from about 2:1 (w/w) to about 7:1 (w/w), optionally wherein the aqueous composition or the reconstituted aqueous composition additionally comprises one or more component selected from poloxamer 338, sodium dihydrogen phosphate monohydrate, citric acid monohydrate, and sodium hydroxide, further optionally wherein the aqueous composition or the reconstituted aqueous composition additionally comprises poloxamer 338, sodium dihydrogen phosphate monohydrate, citric acid monohydrate, and sodium hydroxide.

In a particular embodiment, the aqueous composition or the reconstituted aqueous composition comprises:
rHuPH20;
rilpivirine; and
a sugar or sugar alcohol and an amino acid or a pharmaceutically acceptable salt thereof, wherein the pH of the aqueous composition or the reconstituted aqueous composition is from about 6 to about 6.5, and
wherein the ratio of rilpivirine to the sugar or sugar alcohol and amino acid or a pharmaceutically acceptable salt thereof is from about 1:1 (w/w) to about 20:1 (w/w), optionally wherein the aqueous composition or the reconstituted aqueous composition additionally comprises one or more component selected from poloxamer 338, sodium dihydrogen phosphate monohydrate, citric acid monohydrate, and sodium hydroxide, further optionally wherein the aqueous composition or the reconstituted aqueous composition additionally comprises poloxamer 338, sodium dihydrogen phosphate monohydrate, citric acid monohydrate, and sodium hydroxide.

In a particular embodiment, the aqueous composition or the reconstituted aqueous composition comprises:
rHuPH20;
rilpivirine in the form of particles suspended in the aqueous composition or the reconstituted aqueous composition;
sodium CMC;
sucrose; and
poloxamer 338,
optionally wherein when the rilpivirine has a Dv90 of less than about 1600 nm the ratio of rilpivirine to poloxamer 338 is from about 3:1 (w/w) to about 15:1 (w/w), e.g. about 6:1 (w/w).

In a particular embodiment, the aqueous composition or the reconstituted aqueous composition comprises:
rHuPH20;
rilpivirine in the form of particles suspended in the aqueous composition or the reconstituted aqueous composition;
sodium CMC;
sucrose; and
poloxamer 338,
optionally wherein when the rilpivirine has a Dv90 of from about 1 μm to about 10 μm the ratio of rilpivirine to poloxamer 338 is from about 3:1 (w/w) to about 15:1 (w/w), e.g. about 6:1 (w/w).

In a particular embodiment, the aqueous composition or the reconstituted aqueous composition comprises:
from about 1800 to about 2200 U/mL rHuPH20;
from about 250 to about 350 mg/mL rilpivirine in the form of particles suspended in the aqueous composition or the reconstituted aqueous composition;
from about 1 mg/mL to about 5 mg/mL sodium CMC;
from about 75 mg/mL to about 125 mg/mL sucrose; and from about 5 mg/mL to about 60 mg/mL poloxamer 338,
optionally wherein when the rilpivirine has a Dv90 of less than about 1600 nm the ratio of rilpivirine to poloxamer 338 is from about 3:1 (w/w) to about 15:1 (w/w), e.g. about 6:1 (w/w).

In a particular embodiment, the aqueous composition or the reconstituted aqueous composition comprises:
from about 1800 to about 2200 U/mL rHuPH20;
from about 250 to about 350 mg/mL rilpivirine in the form of particles suspended in the aqueous composition or the reconstituted aqueous composition;
from about 1 mg/mL to about 5 mg/mL sodium CMC;
from about 75 mg/mL to about 125 mg/mL sucrose; and from about 5 mg/mL to about 60 mg/mL poloxamer 338,
optionally wherein when the rilpivirine has a Dv90 of from about 1 μm to about 10 μm the ratio of rilpivirine to poloxamer 338 is from about 3:1 (w/w) to about 15:1 (w/w), e.g. about 6:1 (w/w).

In a particular embodiment, the aqueous composition or the reconstituted aqueous composition comprises:
about 2000 U/mL rHuPH20;
about 300 mg/mL rilpivirine in the form of particles suspended in the aqueous composition or the reconstituted aqueous composition;
about 3 mg/mL sodium CMC;
about 100 mg/mL sucrose; and about 50 mg/mL poloxamer 338.

In a particular embodiment, the aqueous composition or the reconstituted aqueous composition comprises:
about 2000 U/mL rHuPH20;
about 300 mg/mL rilpivirine in the form of particles suspended in the aqueous composition or the reconstituted aqueous composition;
about 3 mg/mL sodium CMC;
about 100 mg/mL sucrose; and about 50 mg/mL poloxamer 338;
wherein the particles have Dv90 of from about 1 μm to about 10 μm.

In a particular embodiment, the aqueous composition or the reconstituted aqueous composition comprises:
about 2000 U/mL rHuPH20;
about 300 mg/mL rilpivirine in the form of particles suspended in the aqueous composition or the reconstituted aqueous composition;
about 3 mg/mL sodium CMC;
about 100 mg/mL sucrose; and
about 50 mg/mL poloxamer 338;
wherein the particles have a Dv90 of less than about 1600 nm.

In a particular embodiment, the aqueous composition or the reconstituted aqueous composition comprises:
from about 1800 to about 2200 U/mL rHuPH20;
from about 250 to about 350 mg/mL rilpivirine in the form of particles suspended in the aqueous composition or the reconstituted aqueous composition;
from about 1 mg/mL to about 5 mg/mL sodium CMC;
from about 75 mg/mL to about 125 mg/mL sucrose; and
from about 5 mg/mL to about 15 mg/mL poloxamer 338,
wherein the particles have Dv90 of from about 1 μm to about 10 μm.

In a particular embodiment, the aqueous composition or the reconstituted aqueous composition comprises:
about 2000 U/mL rHuPH20;
about 300 mg/mL rilpivirine in the form of particles suspended in the aqueous composition or the reconstituted aqueous composition;
about 3 mg/mL sodium CMC;
about 100 mg/mL sucrose; and
about 10 mg/mL poloxamer 338,
wherein the particles have Dv90 of from about 1 μm to about 10 μm.

In a particular embodiment, the aqueous composition or the reconstituted aqueous composition comprises:
about 2000 U/mL rHuPH20;
about 300 mg/mL rilpivirine in the form of particles suspended in the aqueous composition or the reconstituted aqueous composition;
about 3 mg/mL sodium CMC;
about 100 mg/mL sucrose;
about 10 mg/mL poloxamer 338,
about 2 mg/mL NaH₂PO₄·H2O,
about 1 mg/mL citric acid·H2O,
sodium hydroxide (q.s. for pH 6), and
Water (q.s. ad 1 mL),
wherein the particles have Dv90 of from about 1 μm to about 10 μm.

In a particular embodiment, the aqueous composition or the reconstituted aqueous composition comprises:
about 2000 U/mL rHuPH20;
about 300 mg/mL rilpivirine in the form of particles suspended in the aqueous composition or the reconstituted aqueous composition;
about 3 mg/mL sodium CMC;
about 100 mg/mL sucrose;
about 50 mg/mL poloxamer 338,
about 2 mg/mL NaH₂PO₄·H2O,
about 1 mg/mL citric acid·H2O,
sodium hydroxide (q.s. for pH 6), and
Water (q.s. ad 1 mL).

In a particular embodiment, the aqueous composition or the reconstituted aqueous composition comprises:
rHuPH20;
rilpivirine in the form of particles suspended in the aqueous composition or the reconstituted aqueous composition;
sodium CMC;
arginine HCl; and
poloxamer 338,
optionally wherein when the rilpivirine has a Dv90 of less than about 1600 nm the ratio of rilpivirine to poloxamer 338 is from about 3:1 (w/w) to about 15:1 (w/w), e.g. about 6:1 (w/w).

In a particular embodiment, the aqueous composition or the reconstituted aqueous composition comprises:
rHuPH20;
rilpivirine in the form of particles suspended in the aqueous composition or the reconstituted aqueous composition;
sodium CMC;
arginine HCl; and
poloxamer 338,
optionally wherein when the rilpivirine has a Dv90 of from about 1 μm to about 10 μm the ratio of rilpivirine to poloxamer 338 is from about 3:1 (w/w) to about 15:1 (w/w), e.g. about 6:1 (w/w).

In a particular embodiment, the aqueous composition or the reconstituted aqueous composition comprises:
from about 1800 to about 2200 U/mL rHuPH20;
from about 250 to about 350 mg/mL rilpivirine in the form of particles suspended in the aqueous composition or the reconstituted aqueous composition;
from about 1 mg/mL to about 5 mg/mL sodium CMC;
from about 25 mg/mL to about 35 mg/mL arginine HCl; and
from about 5 mg/mL to about 60 mg/mL poloxamer 338,
optionally wherein when the rilpivirine has a Dv90 of less than about 1600 nm the ratio of rilpivirine to poloxamer 338 is from about 3:1 (w/w) to about 15:1 (w/w), e.g. about 6:1 (w/w).

In a particular embodiment, the aqueous composition or the reconstituted aqueous composition comprises:
from about 1800 to about 2200 U/mL rHuPH20;
from about 250 to about 350 mg/mL rilpivirine in the form of particles suspended in the aqueous composition or the reconstituted aqueous composition;
from about 1 mg/mL to about 5 mg/mL sodium CMC;
from about 25 mg/mL to about 35 mg/mL arginine HCl; and from about 5 mg/mL to about 60 mg/mL poloxamer 338,
optionally wherein when the rilpivirine has a Dv90 of from about 1 μm to about 10 μm the ratio of rilpivirine to poloxamer 338 is from about 3:1 (w/w) to about 15:1 (w/w), e.g. about 6:1 (w/w).

In a particular embodiment, the aqueous composition or the reconstituted aqueous composition comprises:
about 2000 U/mL rHuPH20;
about 300 mg/mL rilpivirine in the form of particles suspended in the aqueous composition or the reconstituted aqueous composition;
about 3 mg/mL sodium CMC;
about 30 mg/mL arginine HCl; and
about 50 mg/mL poloxamer 338.

In a particular embodiment, the aqueous composition or the reconstituted aqueous composition comprises:
about 2000 U/mL rHuPH20;
about 300 mg/mL rilpivirine in the form of particles suspended in the aqueous composition or the reconstituted aqueous composition;
about 3 mg/mL sodium CMC;
about 30 mg/mL arginine HCl; and
about 50 mg/mL poloxamer 338;
wherein the particles have Dv90 of from about 1 μm to about 10 μm.

In a particular embodiment, the aqueous composition or the reconstituted aqueous composition comprises:
about 2000 U/mL rHuPH20;
about 300 mg/mL rilpivirine in the form of particles suspended in the aqueous composition or the reconstituted aqueous composition;
about 3 mg/mL sodium CMC;
about 30 mg/mL arginine HCl; and
about 50 mg/mL poloxamer 338;
wherein the particles have a Dv90 of less than about 1600 nm.

In a particular embodiment, the aqueous composition or the reconstituted aqueous composition comprises:
from about 1800 to about 2200 U/mL rHuPH20;
from about 250 to about 350 mg/mL rilpivirine in the form of particles suspended in the aqueous composition or the reconstituted aqueous composition;
from about 1 mg/mL to about 5 mg/mL sodium CMC;
from about 25 mg/mL to about 35 mg/mL arginine HCl; and
from about 5 mg/mL to about 15 mg/mL poloxamer 338,
wherein the particles have Dv90 of from about 1 μm to about 10 μm.

In a particular embodiment, the aqueous composition or the reconstituted aqueous composition comprises:
about 2000 U/mL rHuPH20;
about 300 mg/mL rilpivirine in the form of particles suspended in the aqueous composition or the reconstituted aqueous composition;
about 3 mg/mL sodium CMC;
about 30 mg/mL arginine HCl; and
about 10 mg/mL poloxamer 338,
wherein the particles have Dv90 of from about 1 μm to about 10 μm.

In a particular embodiment, the aqueous composition or the reconstituted aqueous composition comprises:
about 2000 U/mL rHuPH20;
about 300 mg/mL rilpivirine in the form of particles suspended in the aqueous composition or the reconstituted aqueous composition;
about 3 mg/mL sodium CMC;
about 30 mg/mL arginine HCl;
about 10 mg/mL poloxamer 338,
about 2 mg/mL NaH₂PO₄·H2O,
about 1 mg/mL citric acid·H2O,
sodium hydroxide (q.s. for pH 6), and
Water (q.s. ad 1 mL),
wherein the particles have Dv90 of from about 1 μm to about 10 μm.

In a particular embodiment, the aqueous composition or the reconstituted aqueous composition comprises:
about 2000 U/mL rHuPH20;
about 300 mg/mL rilpivirine in the form of particles suspended in the aqueous composition or the reconstituted aqueous composition;
about 3 mg/mL sodium CMC;
about 30 mg/mL arginine HCl;
about 50 mg/mL poloxamer 338,
about 2 mg/mL NaH₂PO₄·H2O,
about 1 mg/mL citric acid·H2O,
sodium hydroxide (q.s. for pH 6), and
Water (q.s. ad 1 mL).

In a particular embodiment, the aqueous composition or the reconstituted aqueous composition comprises:
from about 1800 to about 2200 U/mL rHuPH20;
from about 250 to about 350 mg/mL rilpivirine in the form of particles suspended in the aqueous composition or the reconstituted aqueous composition;
from about 1 mg/mL to about 5 mg/mL sodium CMC;
from about 25 mg/mL to about 35 mg/mL arginine HCl; and
from about 20 mg/mL to about 75 mg/mL poloxamer 338,
optionally wherein when the rilpivirine has a Dv90 of less than about 1600 nm the ratio of rilpivirine to poloxamer 338 is from about 3:1 (w/w) to about 15:1 (w/w), e.g. about 6:1 (w/w).

In a particular embodiment, the aqueous composition or the reconstituted aqueous composition comprises:
from about 1800 to about 2200 U/mL rHuPH20;
from about 250 to about 350 mg/mL rilpivirine in the form of particles suspended in the aqueous composition or the reconstituted aqueous composition;
from about 1 mg/mL to about 5 mg/mL sodium CMC;
from about 25 mg/mL to about 35 mg/mL arginine HCl; and
from about 20 mg/mL to about 75 mg/mL poloxamer 338,
optionally wherein when the rilpivirine has a Dv90 of from about 1 μm to about 10 μm the ratio of rilpivirine to poloxamer 338 is from about 3:1 (w/w) to about 15:1 (w/w), e.g. about 6:1 (w/w).

In a particular embodiment, the aqueous composition or the reconstituted aqueous composition comprises:
about 2000 U/mL rHuPH20;
about 300 mg/mL rilpivirine in the form of particles suspended in the aqueous composition or the reconstituted aqueous composition;
about 3 mg/mL sodium CMC;
about 30 mg/mL arginine HCl; and
about 50 mg/mL poloxamer 338.

In a particular embodiment, the aqueous composition or the reconstituted aqueous composition comprises:
about 2000 U/mL rHuPH20;
about 300 mg/mL rilpivirine in the form of particles suspended in the aqueous composition or the reconstituted aqueous composition;
about 3 mg/mL sodium CMC;
about 30 mg/mL arginine HCl; and
about 50 mg/mL poloxamer 338;
wherein the particles have Dv90 of from about 1 μm to about 10 μm.

In a particular embodiment, the aqueous composition or the reconstituted aqueous composition comprises:
about 2000 U/mL rHuPH20;
about 300 mg/mL rilpivirine in the form of particles suspended in the aqueous composition or the reconstituted aqueous composition;
about 3 mg/mL sodium CMC;
about 30 mg/mL arginine HCl; and
about 50 mg/mL poloxamer 338;
wherein the particles have a Dv90 of less than about 1600 nm.

In a particular embodiment, the aqueous composition or the reconstituted aqueous composition comprises:
from about 1800 to about 2200 U/mL rHuPH20;
from about 250 to about 350 mg/mL rilpivirine in the form of particles suspended in the aqueous composition or the reconstituted aqueous composition;
from about 1 mg/mL to about 5 mg/mL sodium CMC;
from about 75 mg/mL to about 125 mg/mL sucrose; and from about 20 mg/mL to about 75 mg/mL poloxamer 338,
optionally wherein when the rilpivirine has a Dv90 of less than about 1600 nm the ratio of rilpivirine to poloxamer 338 is from about 3:1 (w/w) to about 15:1 (w/w), e.g. about 6:1 (w/w).

In a particular embodiment, the aqueous composition or the reconstituted aqueous composition comprises:
about 2000 U/mL rHuPH20;
about 300 mg/mL rilpivirine in the form of particles suspended in the aqueous composition or the reconstituted aqueous composition;
about 3 mg/mL sodium CMC;
about 100 mg/mL sucrose; and
about 50 mg/mL poloxamer 338.

In a particular embodiment, the aqueous composition or the reconstituted aqueous composition comprises:
about 2000 U/mL rHuPH20;
about 300 mg/mL rilpivirine in the form of particles suspended in the aqueous composition or the reconstituted aqueous composition;
about 3 mg/mL sodium CMC;
about 100 mg/mL sucrose; and
about 50 mg/mL poloxamer 338;
wherein the particles have a Dv90 of less than about 1600 nm.

In a particular embodiment, the aqueous composition or the reconstituted aqueous composition comprises:
about 2000 U/mL rHuPH20;
about 300 mg/mL rilpivirine in the form of particles suspended in the aqueous composition or the reconstituted aqueous composition;
about 3 mg/mL sodium CMC;
about 100 mg/mL sucrose; and
about 50 mg/mL poloxamer 338;
wherein the particles have Dv90 of from about 1 μm to about 10 μm.

The invention also relates to the freeze dried forms corresponding to the particular embodiments above.

In any of the particular embodiments above, the aqueous composition or the reconstituted aqueous composition may additionally further comprise an antioxidant (e.g. methionine), optionally in an amount of from about 1.0 mg/mL to about 2.0 mg/mL (e.g. 1.5 mg/mL).

Unless specified otherwise, in any of the particular embodiments above the particles may have a Dv90 of from about 1 μm to about 10 μm (e.g. about 5 μm to about 6 μm). Alternatively, unless specified otherwise, in any of the particular embodiments above the particles may have a Dv90 of from about 500 nm to about 1600 nm (e.g. about 500 nm to about 700 nm).

The invention also relates to a solid composition comprising rilpivirine or a pharmaceutically acceptable salt thereof and a hyaluronidase. The solid composition may optionally have features (e.g. excipients, amounts and pH) and combinations of features as described herein in relation to the aqueous composition and solid composition of the first aspect of the invention. Thus, purely by way of example, the invention also relates to a solid composition comprising rilpivirine or a pharmaceutically acceptable salt thereof, a hyaluronidase and a cryoprotectant as defined in relation to the first aspect of the invention.

The invention also relates to a solid composition obtainable by freeze drying an aqueous composition comprising rilpivirine or a pharmaceutically acceptable salt thereof, optionally wherein the aqueous composition has features (e.g. excipients, amounts and pH) and combinations of features as described herein in relation to the aqueous composition and solid composition of the first aspect of the invention. The invention also relates to a solid composition comprising rilpivirine or a pharmaceutically acceptable salt thereof: this solid composition may optionally have features (e.g. excipients, amounts and pH) and combinations of features as described herein in relation to the aqueous composition and solid composition in the first aspect of the invention. In an embodiment, this solid composition does not comprise a hyaluronidase. The invention relates to solid compositions having the features of any of the preceding particular embodiments, omitting only the hyaluronidase. The invention also relates to aqueous compositions or reconstituted aqueous compositions according to any of the preceding particular embodiments, omitting only the hyaluronidase.

### Reconstitution of the Solid Composition

The solid composition of the invention may be reconstituted to provide a reconstituted aqueous composition. Thus, the second aspect of the invention relates to a reconstituted aqueous composition obtainable by reconstituting the solid composition as defined herein.

For the avoidance of doubt, the reconstituted aqueous composition comprises the same components in the same amounts as the aqueous composition in the first aspect of the invention that is subsequently freeze dried to provide the solid composition of the first aspect of the invention. Thus, all of the embodiments described in the first aspect of the invention in relation to the aqueous composition apply equally to the reconstituted aqueous composition of the second aspect of the invention. The volume of the reconstituted aqueous composition is not necessarily the same as the volume of the aqueous composition in the first aspect of the invention, so the concentrations of components in these compositions may differ from each other. For example, the aqueous composition in the first aspect of the invention could have a concentration of a component of 300 mg/mL, but if it is reconstituted in double the volume after lyophilisation, the concentration of that component in the reconstituted aqueous composition would be 150 mg/mL.

In an embodiment, the reconstituting comprises adding an aqueous dispersion medium to the solid composition as defined herein, the aqueous dispersion medium being preferably water.

In an embodiment, the dispersion medium comprises one or more other active agents, in particular one or more other antiretroviral agents, in particular one or more other antiretroviral agents of another class, for example an antiretroviral of the INSTI class, for example cabotegravir.

In another embodiment, the aqueous dispersion medium is an aqueous solution of CMC or a pharmaceutically acceptable salt thereof. In another embodiment, the aqueous dispersion medium is an aqueous solution of sodium CMC.

### Lyophilisation

The aqueous composition described herein can be transformed into the solid composition of the invention by freeze drying the aqueous composition using standard procedures known in the art. An example procedure is provided below:

Vials are filled with the aqueous composition. Lyophilisation is performed on a laboratory scale freezedryer (LyoStar3, SP Scientific, USA). Freezing is conducted at −40° C. (shelf inlet temperature) for 60 minutes including equilibrating steps at +5° C. then −5° C. for 15 minutes each. An annealing step at −20° C. (shelf inlet temperature) for 90 minutes is implemented. The shelf temperature ramp rates from the freezing set-point to the primary drying shelf temperature setting are 1° C./min throughout the process. The shelf inlet temperature set-point during primary drying is about −20° C. and secondary drying is about 25° C. The holding time (soak period) of these steps is 600 minutes respectively 60 minutes to allow a modulation in water content in the samples. The chamber pressure during primary and secondary drying is about 100 mTorr for primary drying step and about 300 mTorr for the secondary drying step. Product temperatures during freeze drying are measured using wired thermocouples. Each thermocouple wire is positioned bottom-centre of the vial to obtain both a representative temperature monitoring in the product and an accurate endpoint detection of the ice sublimation phase.

### Uses of the Compositions of the Invention

The reconstituted aqueous composition can be used to treat or prevent HIV infection in a subject.

Thus, in a third aspect the invention relates to a method for the treatment or prevention of HIV infection in a subject, the method comprising administering to the subject the reconstituted aqueous composition as defined herein. In a fourth aspect the invention relates to the reconstituted aqueous composition as defined herein for use in the treatment or prevention of HIV infection in a subject. In a fifth aspect the invention relates to use of the reconstituted aqueous composition as defined in herein for the manufacture of a medicament for treating or preventing HIV infection in a subject. In a sixth aspect there is provided a solid composition as defined herein for use in the treatment or prevention of HIV infection in a subject. In an seventh aspect there is provided the use of the solid composition as defined herein for the manufacture of a medicament for treating or preventing HIV infection in a subject.

In a preferred embodiment, the subject is a human.

In an embodiment, the reconstituted aqueous composition is administered to the subject intermittently such that the time interval between administrations (i.e. the dosing interval) is about three months to about two years. In an embodiment, the time interval is about three months to about eighteen months. In an embodiment, the time interval is about three months to about one year. In an embodiment, the time interval is about three months to about six months. In an embodiment, the time interval is about six months to about one year. In an embodiment, the time interval is about one year. In an embodiment, the time interval is about three months. In an embodiment, the time interval is about six months.

In an embodiment, the reconstituted aqueous composition is administered to the subject intermittently such that the time interval between administrations (i.e. the dosing interval) is of about three months, or about four months, or about five months or of about six months or of about seven months or of about eight months or of about nine months or of about ten months or of about eleven months or of about one year or of about one year to about two years.

In an embodiment, the reconstituted aqueous composition is administered by subcutaneous injection or intramuscular injection. In an embodiment, the reconstituted aqueous composition is administered by intramuscular injection. In a preferred embodiment, the reconstituted aqueous composition is administered by subcutaneous injection.

In another embodiment, the reconstituted aqueous composition is administered by a manual injection process.

The rilpivirine or pharmaceutically acceptable salt thereof is present in the reconstituted aqueous composition (and is administered to the subject) in a therapeutically effective amount. By “therapeutically effective amount” it is meant an amount sufficient to provide a therapeutic effect.

In an embodiment, each administration comprises up to about 150 mL of the reconstituted aqueous composition described herein. In another embodiment, each administration comprises from about 3 mL to about 150 mL of the reconstituted aqueous composition. In another embodiment, each administration comprises from about 3 mL to about 100 mL of the reconstituted aqueous composition. In another embodiment, each administration comprises from about 3 mL to about 25 mL of the reconstituted aqueous composition. In another embodiment, each administration comprises from about 3 mL to about 15 mL of the reconstituted aqueous composition. In another embodiment, each administration comprises from about 5 mL to about 25 mL of the reconstituted aqueous composition. In another embodiment, each administration comprises from about 6 mL to about 20 mL of the reconstituted aqueous composition. In another embodiment, each administration comprises from about 6 mL to about 18 mL of the reconstituted aqueous composition. In another embodiment, each administration comprises from about 6 mL to about 15 mL of the reconstituted aqueous composition. In another embodiment, each administration comprises from about 6 mL to about 12 mL of the reconstituted aqueous composition. In another embodiment, each administration comprises from about 9 mL to about 18 mL of the reconstituted aqueous composition. In another embodiment, each administration comprises from about 9 mL to about 15 mL of the reconstituted aqueous composition. In another embodiment, each administration comprises from about 9 mL to about 12 mL of the reconstituted aqueous composition. In another embodiment, each administration comprises about 3 mL of the reconstituted aqueous composition. In another embodiment, each administration comprises about 4 mL of the reconstituted aqueous composition. In another embodiment, each administration comprises about 5 mL of the reconstituted aqueous composition. In another embodiment, each administration comprises about 6 mL of the reconstituted aqueous composition. In another embodiment, each administration comprises about 7 mL of the reconstituted aqueous composition. In another embodiment, each administration comprises about 8 mL of the reconstituted aqueous composition. In another embodiment, each administration comprises about 9 mL of the reconstituted aqueous composition. In another embodiment, each administration comprises about 12 mL of the reconstituted aqueous composition. In another embodiment, each administration comprises about 15 mL of the reconstituted aqueous composition. In another embodiment, each administration comprises about 18 mL of the reconstituted aqueous composition. In a preferred embodiment, each administration comprises about 3 mL, about 7 mL, or about 15 mL of the reconstituted aqueous composition.

In an embodiment, for the treatment of HIV infection, the dose of rilpivirine to be administered may be calculated on a basis of from about 300 mg to about 1200 mg/month, or from about 450 mg to about 1200 mg/month, or from about 450 mg to about 900 mg/month, or from about 600 mg to about 900 mg/month, or from about 450 mg to about 750 mg/month, or about 450 mg/month, or about 600 mg/month, or about 750 mg/month, or about 900 mg/month. Doses for other dosing regimens can readily be calculated by multiplying the monthly dose with the number of months between each administration. For example, in case of a dose of 450 mg/month, and in case of a time interval of 6 months between each administration, the dose of rilpivirine to be administered in each administration is 2700 mg. The indicated “mg” corresponds to mg of rilpivirine (i.e. rilpivirine in its free base form). Thus, by way of example, 1 mg of rilpivirine (i.e. rilpivirine in its free base form) corresponds to 1.1 mg of rilpivirine hydrochloride.

In an embodiment, for the treatment of HIV infection, the dose of rilpivirine to be administered may be calculated on a basis of from about 300 mg to about 1200 mg/4 weeks (28 days), or from about 450 mg to about 1200 mg/4 weeks (28 days), or from about 450 mg to about 900 mg/4 weeks (28 days), or from about 600 mg to about 900 mg/4 weeks (28 days), or from about 450 mg to about 750 mg/4 weeks (28 days) or about 450 mg/4 weeks (28 days), or about 600 mg/4 weeks (28 days), or about 750 mg/4 weeks (28 days) or about 900 mg/4 weeks (28 days). Doses for other dosing regimens can readily be calculated by multiplying the week or day dose with the number of weeks between each administration. For example, in case of a dose of 450 mg/4 weeks (28 days), and in case of a time interval of 24 weeks between each administration, the dose of rilpivirine to be administered in each administration is 2700 mg. Or for example, in case of a dose of 750 mg/4 weeks (28 days), and in case of a time interval of 24 weeks between each administration, the dose of rilpivirine to be administered in each administration is 4500 mg. The indicated “mg” corresponds to mg of rilpivirine (i.e. rilpivirine in its free base form). Thus, by way of example, 1 mg of rilpivirine (i.e. rilpivirine in its free base form) corresponds to 1.1 mg of rilpivirine hydrochloride.

In an embodiment, the rilpivirine or pharmaceutically acceptable salt thereof in the reconstituted aqueous composition is used in an amount such that the blood plasma concentration of rilpivirine in the subject is kept at a level above about 12 ng/ml, preferably ranging from about 12 ng/ml to about 100 ng/ml, more preferably from about 12 ng/ml to about 50 ng/ml for at least 3 months after administration, or at least 6 months after administration, or at least 9 months after administration, or at least 1 year after administration, or at least 2 years after each administration. In a preferred embodiment, the rilpivirine or pharmaceutically acceptable salt thereof in the reconstituted aqueous composition is used in an amount such that the blood plasma concentration of rilpivirine in the subject is kept at a level of from about 12 ng/ml to about 100 ng/ml for at least 6 months.

In the instance of prevention of HIV infection, each administration of rilpivirine or pharmaceutically acceptable salt thereof may comprise the same dosing as for therapeutic applications as described above.

In an embodiment, the reconstituted aqueous composition is used in combination with one or more other active agents, in particular one or more other antiretroviral agents, in particular one or more other antiretroviral agents of another class, such as for example an antiretroviral of the INSTI class, such as for example cabotegravir.

In an embodiment, said one or more other antiretroviral agents, e.g. cabotegravir, is administered as an intramuscular or subcutaneous injection, in particular as an injectable micro- or nanosuspension, at a time interval of about three months to about two years. In an embodiment, said one or more other antiretroviral agent, e.g. cabotegravir, is administered at the same intermittent time interval as reconstituted aqueous composition as described herein, e.g. the reconstituted aqueous composition and the other antiretroviral agent are administered intermittently at a time interval of about three months, or of about four months, or of about five months or of about six months or of about seven months or of about eight months or of about ten months or of about eleven months or of about one year or of about one year to about 2 years. In an embodiment the reconstituted aqueous composition and the one or more other antiretroviral agents, e.g. cabotegravir, are administered simultaneously or sequentially by intramuscular or subcutaneous injection, in particular subcutaneous injection. In an embodiment, the reconstituted aqueous composition and the one or more other antiretroviral agents, e.g. cabotegravir, are administered simultaneously, in particular by subcutaneous injection. In an embodiment, the reconstituted aqueous composition and the one or more other antiretroviral agents, e.g. cabotegravir, are administered sequentially, in particular by subcutaneous injection. In an embodiment, the reconstituted aqueous composition is administered first followed by a cabotegravir injection.

As used herein the term “treatment of HIV infection” relates to the treatment of a subject infected with HIV. The term “treatment of HIV infection” also relates to the treatment of diseases associated with HIV infection, for example AIDS, or other conditions associated with HIV infection including thrombocytopaenia, Kaposi's sarcoma and infection of the central nervous system characterized by progressive demyelination, resulting in dementia and symptoms such as, progressive dysarthria, ataxia and disorientation, and further conditions where HIV infection has also been associated with, such as peripheral neuropathy, progressive generalized lymphadenopathy (PGL), and AIDS-related complex (ARC).

As used herein the term “prevention of HIV infection” relates to the prevention or avoidance of a subject (who is not infected with HIV) becoming infected with HIV. The source of infection can be various, a material containing HIV, in particular a body fluid that contains HIV such as blood or semen, or another subject who is infected with HIV. Prevention of HIV infection relates to the prevention of the transmission of the virus from the material containing HIV or from the HIV infected individual to an uninfected person, or relates to the prevention of the virus from entering the body of an uninfected person. Transmission of the HIV virus can be by any known cause of HIV transfer such as by sexual transmission or by contact with blood of an infected subject, e.g. medical staff providing care to infected subjects. Transfer of HIV can also occur by contact with HIV infected blood, e.g. when handling blood samples or with blood transfusion. It can also be by contact with infected cells, e.g. when carrying out laboratory experiments with HIV infected cells.

The term “treatment of HIV infection” refers to a treatment by which the viral load of HIV (represented as the number of copies of viral RNA in a specified volume of serum) is reduced. The more effective the treatment, the lower the viral load. Preferably the viral load should be reduced to as low levels as possible, e.g. below about 200 copies/mL, in particular below about 100 copies/mL, more in particular below 50 copies/mL, if possible below the detection limit of the virus. Reductions of viral load of one, two or even three orders of magnitude (e.g. a reduction in the order of about 10 to about 10², or more, such as about 103) are an indication of the effectiveness of the treatment. Another parameter to measure effectiveness of HIV treatment is the CD4 count, which in normal adults ranges from 500 to 1500 cells per μL. Lowered CD4 counts are an indication of HIV infection and once below about 200 cells per μL, AIDS may develop. An increase of CD4 count, e.g. with about 50, 100, 200 or more cells per μL, is also an indication of the effectiveness of anti-HIV treatment. The CD4 count in particular should be increased to a level above about 200 cells per μL, or above about 350 cells per μL. Viral load or CD4 count, or both, can be used to diagnose the degree of HIV infection. Another parameter to measure effectiveness of HIV treatment is keeping the HIV-infected subject virologically suppressed (HIV-1 RNA<50 copies/mL) when on the treatment according to the present invention.

The term “treatment of HIV infection” and similar terms refer to that treatment that lowers the viral load, increases CD4 count, or both, or keeps the HIV-infected subject virologically suppressed, as described above. The term “prevention of HIV infection” and similar terms refer to that situation where there is a decrease in the relative number of newly infected subjects in a population in contact with a source of HIV infection such as a material containing HIV, or a HIV infected subject. Effective prevention can be measured, for example, by measuring in a mixed population of HIV infected and non-infected individuals, if there is a decrease of the relative number of newly infected individuals, when comparing non-infected individuals treated with a pharmaceutical composition of the invention, and non-treated non-infected individuals. This decrease can be measured by statistical analysis of the numbers of infected and non-infected individuals in a given population over time.

### Kits

In an eighth aspect there is provided a kit comprising: (i) the solid composition of the invention as described herein, and (ii) a diluent, in particular a diluent to reconstitute the solid composition (i).

In an ninth aspect there is provided a kit comprising: (i) the solid composition of the invention as described herein, (ii) a composition comprising one or more other active agents, in particular one or more other antiretroviral agents, in particular one or more other antiretroviral agents of another class, such as for example an antiretroviral of the INSTI class, such as for example cabotegravir, and optionally (iii) a diluent, in particular a diluent to reconstitute the solid composition (i).

### General Definitions

The term “comprising” encompasses “including” as well as “consisting”, e.g. a composition “comprising” X may consist exclusively of X or may include something additional, e.g. X+Y. The term “comprising” used herein also encompasses “consisting essentially of”, e.g. a composition “comprising” X may consist of X and any other components that do not materially affect the essential characteristics of the composition.

The term “about” in relation to a numerical value Y is optional and means, for example, Y±10%.

When a time interval is expressed as a specified number of months, it runs from a given numbered day of a given month to the same numbered day of the month that falls the specified number of months later. Where the same numbered day does not exist in the month that falls the specified number of months later, the time interval runs into the following month for the same number of days it would have run if the same numbered day would exist in the month that falls the specified number of months later.

When a time interval is expressed as a number of years, it runs from a given date of a given year to the same date in the year that falls the specified number of years later. Where the same date does not exist in the year that falls the specified number of years later, the time interval runs for the same number of days it would have run if the same numbered day would exist in the month that falls the specified number of months later. In other words, if the time interval starts on 29th February of a given year but ends in a year where there is no 29th February, the time period ends instead on 1st March in that year. The term “about” in relation to such a definition means that the time interval may end on a date that is ±10% of the time interval.

In an embodiment, the time interval may start up to 7 days before or after the start of the time interval and end up to 7 days before or after the end of the time interval.

All references cited herein are incorporated by reference in their entirety.

The invention will now be described with reference to the following examples. For the avoidance of doubt, these examples do not limit the scope of the invention. Modifications may be made whilst remaining within the scope and spirit of the invention.

### EXAMPLES

### Example 1—Melting Temperature Studies Under Different Storage Conditions

This example compares the Tₘ of rHuPH20 in various compositions, each having a different combination of excipients and/or pH, under different storage conditions.

A higher Tₘ indicates higher thermal stability. A Tₘ of from about 25 to about 30° C. higher than the intended long term storage conditions is targeted to provide optimal thermodynamic stability of the hyaluronidase.

Compositions 1-21 were prepared by adding the component(s) shown in Table 1 below to the following composition A:
Glucose monohydrate (19.25 mg/mL)
rHuPH20 (2000 U/mL)
Sodium dihydrogen phosphate monohydrate (2 mg/mL)
Citric acid monohydrate (1 mg/mL)
Poloxamer 338 (50 mg/mL)
Sodium hydroxide (varied to provide target pH)
Water for injection (q.s. ad 1 mL)

The pH of the composition was adjusted to 5.0, 6.0, 6.5, or 7.0 as necessary by varying the amount of the sodium hydroxide.

TABLE 1
Tₘ studies

| Composition | pH | Additional component 1 | Additional component 2 |
|---|---|---|---|
| 1 | 5.0 | — | — |
| 2 | 5.0 | Mannitol (50 mg/mL) | — |
| 3 | 5.0 | Sucrose (50 mg/mL) | — |
| 4 | 5.0 | Histidine (25 mg/mL) | — |
| 5 | 5.0 | Glycine (25 mg/mL) | — |
| 6 | 5.0 | Glycine (10 mg/mL) | Sucrose (50 mg/mL) |
| 7 | 5.0 | Glycine (10 mg/mL) | Mannitol (25 mg/mL) |
| 8 | 6.0 | — | — |
| 9 | 6.0 | Mannitol (50 mg/mL) | — |
| 10 | 6.0 | Sucrose (50 mg/mL) | — |
| 11 | 6.0 | Histidine (25 mg/mL) | — |
| 12 | 6.0 | Glycine (25 mg/mL) | — |
| 13 | 6.0 | Glycine (10 mg/mL) | Sucrose (50 mg/mL) |
| 14 | 6.0 | Glycine (10 mg/mL) | Mannitol (25 mg/mL) |
| 15 | 6.5 | Glycine (10 mg/mL) | Sucrose (50 mg/mL) |
| 16 | 7.0 | Histidine (25 mg/mL) | — |
| 17 | 7.0 | Glycine (10 mg/mL) | Sucrose (50 mg/mL) |
| 18 | 7.0 | Glycine (10 mg/mL) | Mannitol (25 mg/mL) |
| 19 | 7.0 | Glycine (15 mg/mL) | — |
| 20 | 7.0 | Glycine (10 mg/mL) | — |
| 21 | 7.0 | Glycine (5 mg/mL) | — |

The Tₘ values in this example were measured using nano differential scanning fluorimetry (nDSF) assays. nDSF uses the intrinsic tryptophan and tyrosine fluorescence to monitor protein unfolding. The nDSF assay was as follows.

A Prometheus NT.Plex instrument controlled by PR.ThermControl-CFR software was used to run all nDSF assays. A temperature range of 20-95° C. with a temperature gradient of 1° C./min was used when measuring the samples. An excitation power of 95% was used in all cases. The instrument determined the fluorescence ratio of 350 nm (tryptophan) and 330 nm (tyrosine) of the protein during a heat ramp to 95° C. The unfolding onset temperature (Tₒₙ) and the inflection points (Tₘ) of the present unfolding transitions were then calculated from the unfolding curve.

To enable high throughput sample testing, the nanoDSF instrument was operated in combination with a robotic autosampler. The autosampler performs loading and transfer of the capillary chips from a 384-well plate for nanoDSF measurements. The preparation of the 384-well plate was automated using a Tecan Fluent 760 liquid handling system in combination with custom-made sample holders for 2R vials that were typically provided for sample testing.

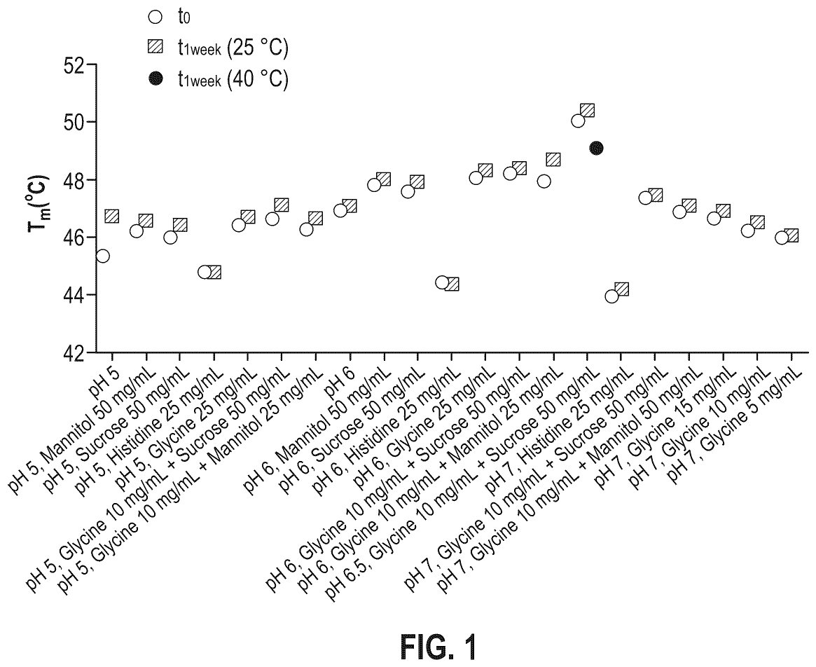

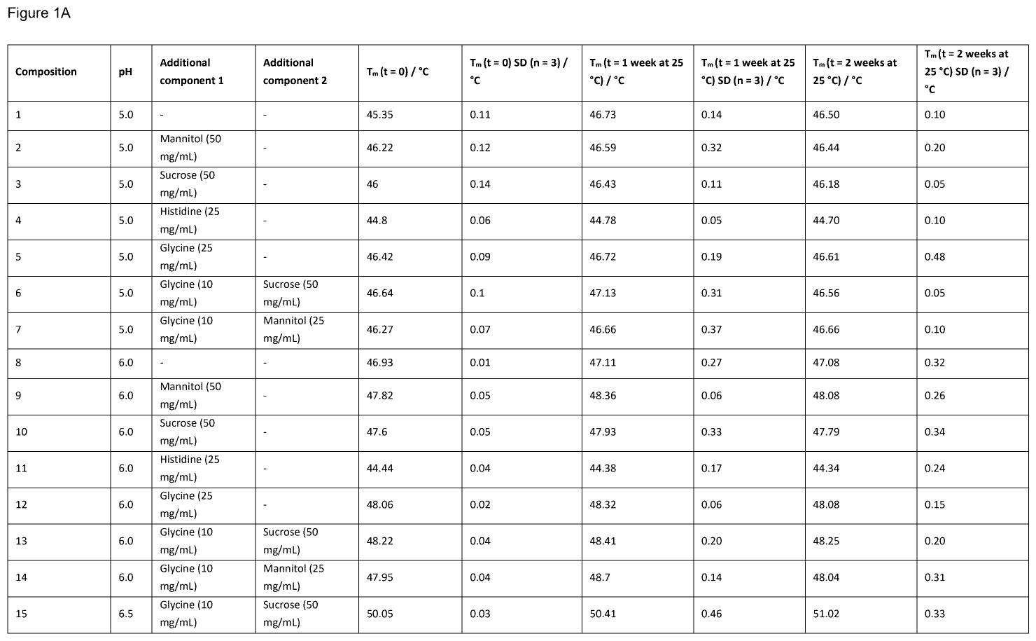

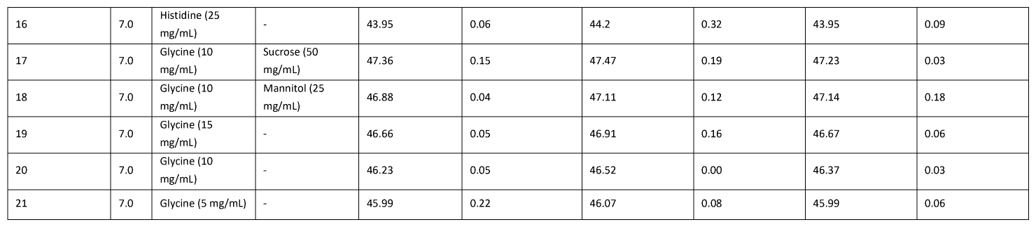

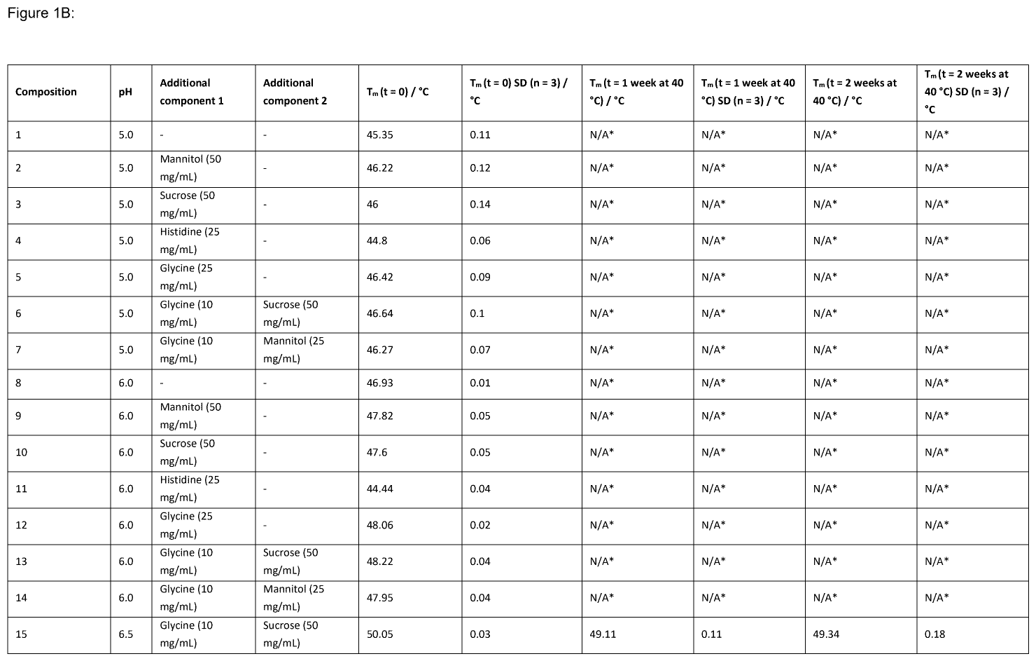

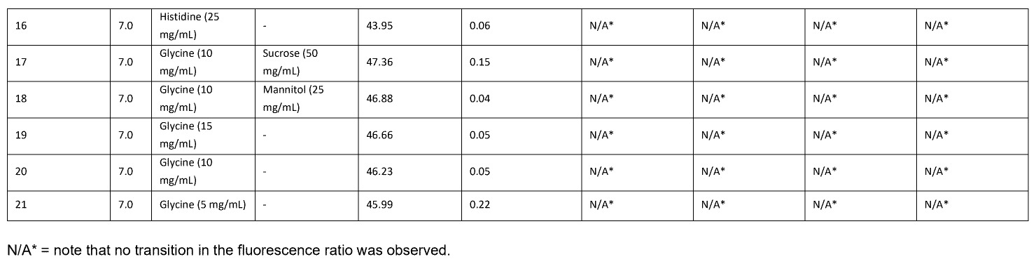

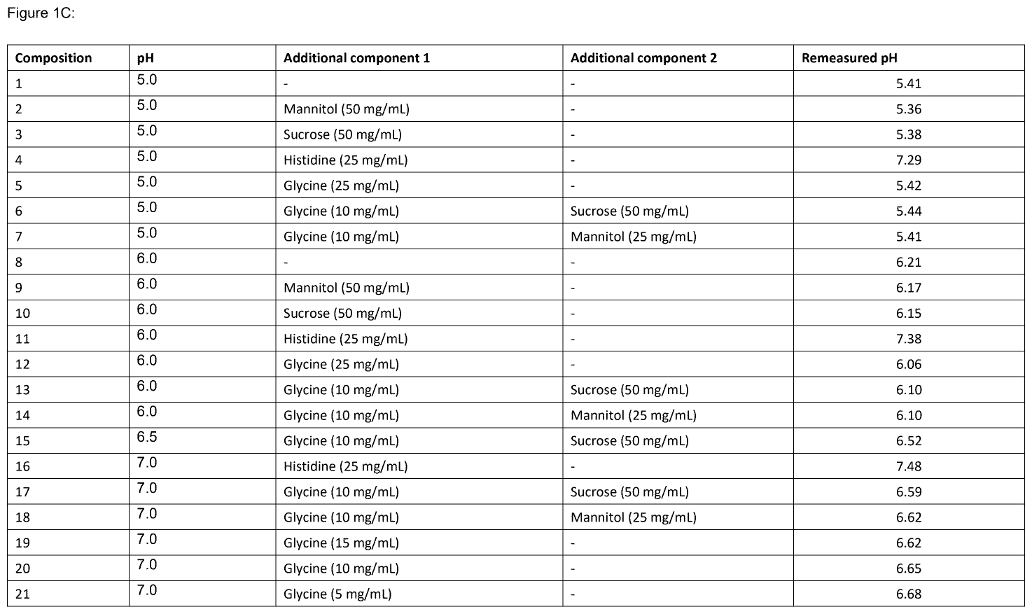

Compositions 1-21 were prepared as set out above and then stored in a refrigerator at 5° C. for between 2 days and 2 weeks. The Tₘ was then measured straight away, after 1 week's storage at 25° C., 2 weeks' storage at 25° C., and at extreme storage conditions, i.e. 1 week's storage at 40° C. and 2 weeks' storage at 40° C. The pH of the aqueous compositions was remeasured immediately before the Tₘ values were measured. The results are shown in FIG. 1, FIG. 1A, FIG. 1B, and FIG. 1C.

### Example 2a—Melting Temperature Studies

This example compares the Tₘ of rHuPH20 in various compositions, each having a different combination of excipients and/or pH.

A composition B comprising the following excipients was prepared:
Sodium dihydrogen phosphate monohydrate (2 mg/mL)
Citric acid monohydrate (1 mg/mL)
rHuPH20 (2000 U/mL)
Sodium hydroxide (varied to provide target pH)
Water for injection (q.s. ad 1 mL)

Compositions 22-44 were prepared by adding the component(s) shown in Table 2 below to composition B, and the pH was adjusted to 6.0, 6.5, or 7.0 by varying the amount of the sodium hydroxide in the composition.

TABLE 2
Tₘ studies

| Composition | Additional component | pH | Tₘ/° C. |
|---|---|---|---|
| 22 | — | 6.5 | 48.44 |
| 23 | — | 7 | 47.6 |
| 24 | Glycine (10 mg/mL) | 6 | 48.79 |
| 25 | Glycine (10 mg/mL) | 6.5 | 47.83 |
| 26 | Glycine (10 mg/mL) | 7 | 46.99 |
| 27 | Glycine (25 mg/mL) | 6 | 49.74 |
| 28 | Glycine (25 mg/mL) | 6.5 | 47.47 |
| 29 | Glycine (25 mg/mL) | 7 | 47.07 |
| 30 | Histidine (10 mg/mL) | 6 | 50.64 |
| 31 | Histidine (10 mg/mL) | 6.5 | 47.45 |
| 32 | Histidine (10 mg/mL) | 7 | 49.73 |
| 33 | Arginine (10 mg/mL) | 6 | 52.47 |
| 34 | Arginine (10 mg/mL) | 6.5 | 52.7 |
| 35 | Arginine (10 mg/mL) | 7 | 51.76 |
| 36 | Mannitol (50 mg/mL) | 6 | 48.87 |
| 37 | Mannitol (50 mg/mL) | 6.5 | 47.71 |
| 38 | Mannitol (50 mg/mL) | 7 | 48.23 |
| 39 | Sucrose (50 mg/mL) | 6 | 50.27 |
| 40 | Sucrose (50 mg/mL) | 6.5 | 48.41 |
| 41 | Sucrose (50 mg/mL) | 7 | 46.79 |
| 42 | Sodium CMC (25 mg/mL) | 6 | 54.1 |
| 43 | Sodium CMC (25 mg/mL) | 6.5 | 54.06 |
| 44 | Sodium CMC (25 mg/mL) | 7 | 50.41 |

Compositions 22-44 were prepared as set out above and then stored in a refrigerator at 5° C. for between 2 days and 2 weeks. The Tₘ for rHuPH20 was then measured immediately for each composition using the method described in Example 1.

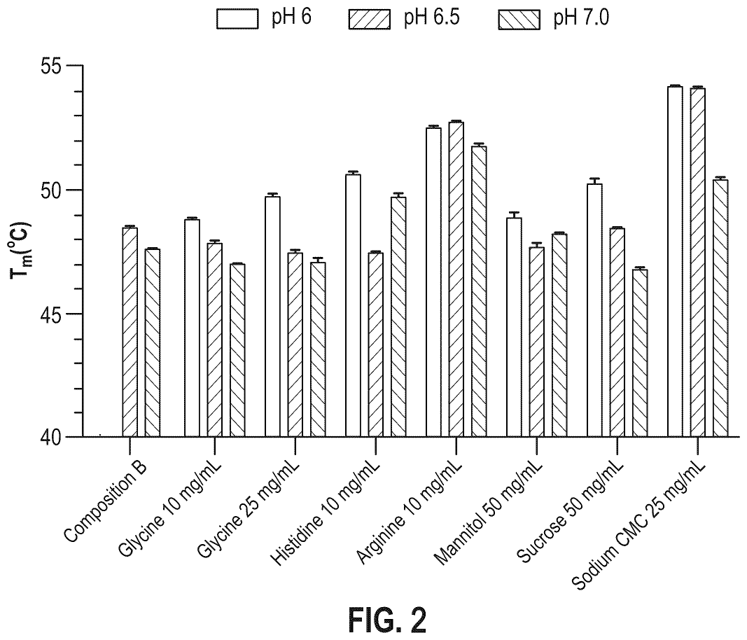

The results are shown in Table 2 and FIG. 2.

### Example 2b—Enzyme Stability Studies Under Different Storage Conditions

This example compares the fluorescence emission spectra of rHuPH20 compositions after storage under different conditions. Fluorescence emission spectroscopy uses a beam of light which is absorbed by the enzyme (rHuPH20), and the fluorophore in the ground state is excited to a higher energy state, thereby resulting in emission. For an enzyme such as rHuPH20, the fluorescence of tryptophan may be used to measure a change in tertiary enzyme structure during or after storage under different conditions, and thus quantify the thermal stability of the rHuPH20. The maximum emission wavelengths for folded and unfolded rHuPH20 are 330 nm and 350 nm, respectively.

The spectra of the compositions were collected immediately after storage at 5° C. and after storage at 40° C. for one and two weeks. In addition to peak maximum and minimum, the intensity ratio 350/330 nm was calculated at each time point and plotted as a function of storage time. The slope of this curve was used to have a semi-quantitative thermal stability assessment and comparison among compositions.

### Composition Preparation

200 μl of each composition was dispensed manually into an UV-STAR*, COC, 96-well flat-bottom microplate.

The fluorescence emission spectrum of the samples was measured before and after stressing via a Tecan plate reader (Infinite 200). The following method parameters were used:

### Temperature:

Parameter (on), temperature=25.0° C.

### Wait for Temperature:

Parameter minimum=24.5° C.; maximum=25.5° C.

### Fluorescence Intensity Scan:

Scan selection (emission scan)
Mode (top)
Excitation wavelengths: from =280 nm; bandwidth: 230 . . . 315: 5 nm; 316 . . . 850: 9 nm
Emission wavelengths: from =310 nm; to =420 nm; step=1 nm; bandwidth: 280 . . . 850: 20 nm; 111 measurements
Integration: lag time=0 μs; integration time=20 μs
Read: number of flashes=25; settle time=0 ms
Gain (manual=95)
Label: name=Label1

The results are shown in Table 3, below.

TABLE 3
Slope of the ratio between 350 nm/330 nm versus time

| Composition | Additional component | pH | Slope 350 nm/330 nm |
|---|---|---|---|
| 22 | — | 6.5 | |
| 23 | — | 7 | |
| 24 | Glycine (10 mg/mL) | 6 | 0.004300 |
| 25 | Glycine (10 mg/mL) | 6.5 | 0.005389 |
| 26 | Glycine (10 mg/mL) | 7 | 0.006196 |
| 27 | Glycine (25 mg/mL) | 6 | 0.001013 |
| 28 | Glycine (25 mg/mL) | 6.5 | 0.004787 |
| 29 | Glycine (25 mg/mL) | 7 | 0.002456 |
| 30 | Histidine (10 mg/mL) | 6 | 0.004556 |
| 31 | Histidine (10 mg/mL) | 6.5 | 0.005702 |
| 32 | Histidine (10 mg/mL) | 7 | 0.004277 |
| 33 | Arginine (10 mg/mL) | 6 | 0.002788 |
| 34 | Arginine (10 mg/mL) | 6.5 | 0.001392 |
| 35 | Arginine (10 mg/mL) | 7 | 0.002748 |
| 36 | Mannitol (50 mg/mL) | 6 | 0.004070 |
| 37 | Mannitol (50 mg/mL) | 6.5 | 0.005511 |
| 38 | Mannitol (50 mg/mL) | 7 | 0.005598 |
| 39 | Sucrose (50 mg/mL) | 6 | 0.001455 |
| 40 | Sucrose (50 mg/mL) | 6.5 | 0.002963 |
| 41 | Sucrose (50 mg/mL) | 7 | 0.006229 |
| 42 | Sodium CMC (25 mg/mL) | 6 | −0.00035 |
| 43 | Sodium CMC (25 mg/mL) | 6.5 | 0.001466 |
| 44 | Sodium CMC (25 mg/mL) | 7 | 0.003267 |

### Example 3—Melting Temperature Studies Under Different Storage Conditions

This example compares the melting temperatures of rHuPH2O in various compositions under different storage conditions.

A composition C comprising the following excipients was prepared:
rHuPH2O (2000 U/mL)
Sodium dihydrogen phosphate monohydrate (2 mg/mL)
Citric acid monohydrate (1 mg/mL)
Poloxamer 338 (50 mg/mL)
Sodium hydroxide (q.s. for pH 6.0)
Water for injection (q.s. ad 1 mL)

Compositions 45-57 were then prepared by adding the component(s) as shown in Table 4 to the composition C, and the pH was adjusted to 6.0 by varying the amount of sodium hydroxide in the composition.

TABLE 4
Tₘ under different storage conditions

| | | | | | | Tₘ (t = 1 |
|---|---|---|---|---|---|---|
| | | | | | Tₘ (t = 1 | week at |
| | | | | Tₘ (t = 0) | week at | 40° C.) |
| | Additional | Additional | Tₘ (t = 0)/ | SD (n = 3)/ | 40° C.)/ | SD (n = 3)/ |
| Composition | component 1 | component 2 | ° C. | ° C./° C. | ° C. | ° C. |
| 45 | Sucrose (100 | — | 48.31 | 0.05 | — | — |
| | mg/mL) | | | | | |
| 46 | Mannitol (50 | — | 47.65 | 0.26 | — | — |
| | mg/mL) | | | | | |
| 47 | Glucose (50 | — | 47.65 | 0.09 | — | — |
| | mg/mL) | | | | | |
| 48 | Arginine (5 | — | 54.99 | 0.06 | 54.93 | 0.01 |
| | mg/mL) | | | | | |
| 49 | Arginine (25 | — | 52.74 | 0.07 | 52.91 | 0.15 |
| | mg/mL) | | | | | |
| 50 | Sucrose (100 | Glycine (1 | 48.62 | 0.14 | — | — |
| | mg/mL) | mg/mL) | | | | |
| 51 | Sucrose (100 | Glycine (5 | 48.86 | 0.05 | — | — |
| | mg/mL) | mg/mL) | | | | |
| 52 | Sucrose (100 | Arginine (5 | 53.67 | 0.04 | 53.72 | 0.09 |
| | mg/mL) | mg/mL) | | | | |
| 53 | Sucrose (100 | Sodium CMC | 54.86 | 0.04 | 55.28 | 0.25 |
| | mg/mL) | (10 mg/mL) | | | | |
| 54 | Mannitol (50 | Glycine (5 | 48.13 | 0.15 | — | — |
| | mg/mL) | mg/mL) | | | | |
| 55 | Mannitol (50 | Arginine (5 | 53.03 | 0.09 | 53.11 | 0.16 |
| | mg/mL) | mg/mL) | | | | |
| 56 | Mannitol (50 | Sodium CMC | 54.38 | 0.05 | 54.54 | 0.12 |
| | mg/mL) | (5 mg/mL) | | | | |
| 57 | Mannitol (50 | Sodium CMC | | | | |
| | mg/mL) | (10 mg/mL) | 54.06 | 0.05 | 54.18 | 0.07 |

Compositions 45-57 were prepared as set out above and then stored in a refrigerator at 5° C. for between 2 days and 2 weeks. The Tₘ values were measured straight away (i.e. “t=0”) and after storage for 1 week at 40° C. using the method as described in Example 1.

The pH of the compositions was remeasured immediately before the Tₘ values were measured.

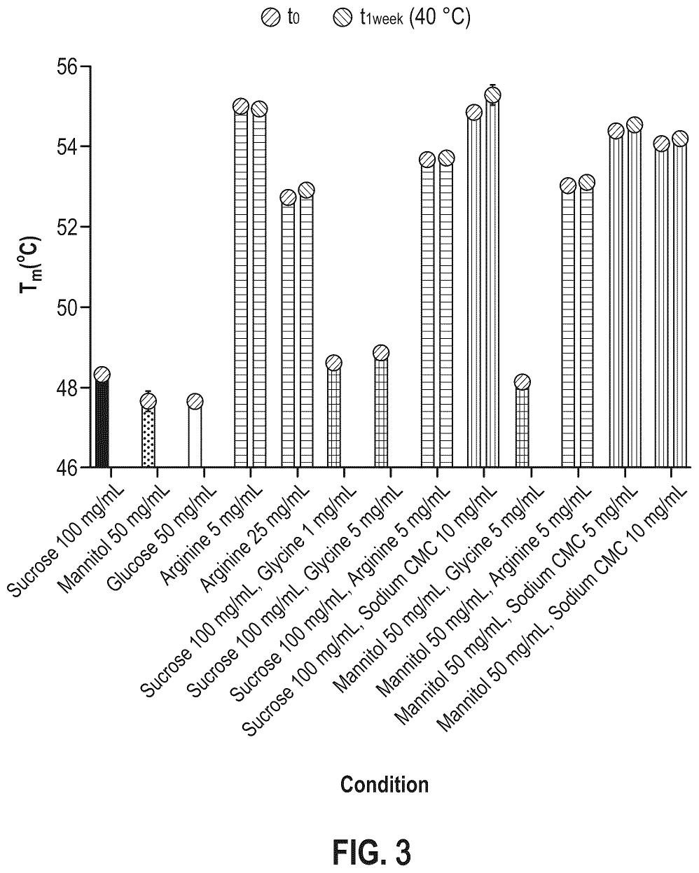

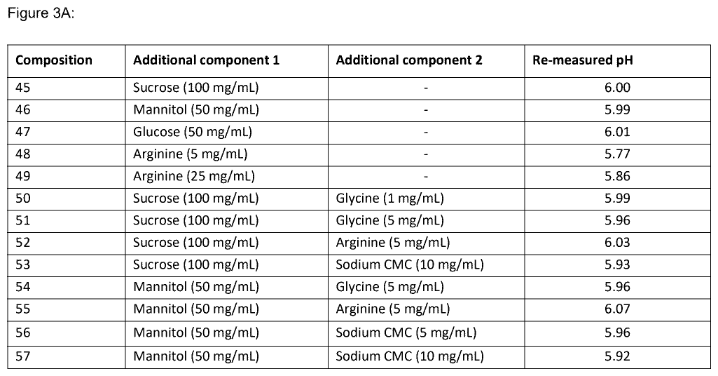

The results are shown in Table 4 and FIG. 3 and FIG. 3A. Where a Tₘ value at t=1 week at 40° C. is not displayed (i.e. “-” for Tm and its SD), the rHuPH20 had decomposed.

### Example 4—Sodium CMC Concentration

This example examines the dependence of Tₘ on sodium CMC concentration.

A composition D comprising the following excipients was prepared:
rHuPH20 (2000 U/mL)
Sodium dihydrogen phosphate monohydrate (2 mg/mL)
Citric acid monohydrate (1 mg/mL)
Poloxamer 338 (50 mg/mL)
Sodium hydroxide (q.s. for pH 6.0)
Mannitol (50 mg/mL) or sucrose (100 mg/mL)
Water for injection (q.s. ad 1 mL)

Compositions 58-63 were then prepared by adding sodium CMC shown in Table 5 below to composition D, and the pH was adjusted to 6.0 by varying the amount of sodium hydroxide in the composition.

TABLE 5
Sodium CMC concentration

| | Sodium CMC | |
|---|---|---|
| Composition | concentration/mg/mL | Sugar/sugar alcohol |
| 58 | 5 | Mannitol (50 mg/mL) |
| 59 | 5 | Sucrose (100 mg/mL) |
| 60 | 10 | Mannitol (50 mg/mL) |
| 61 | 10 | Sucrose (100 mg/mL) |
| 62 | 15 | Mannitol (50 mg/mL) |
| 63 | 15 | Sucrose (100 mg/mL) |

Compositions 58-63 were prepared as set out above and then stored in a refrigerator at 5° C. for between 2 days and 2 weeks. Tₘ values were then measured using the method described in Example 1.

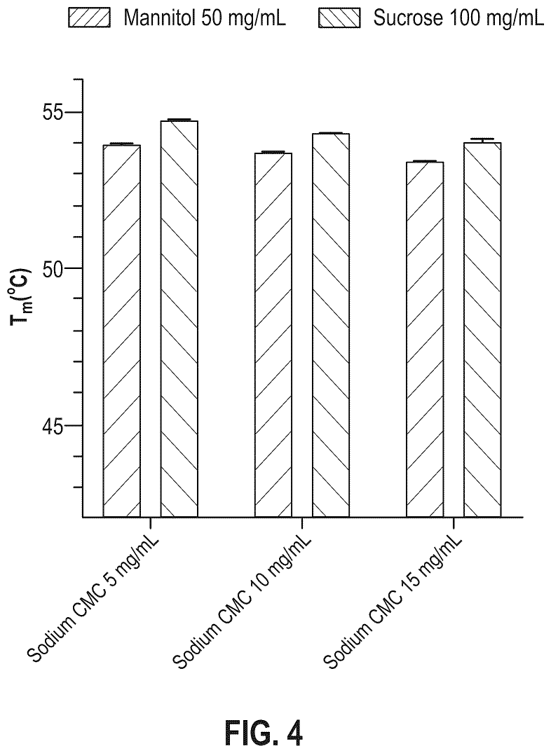

The results are shown in FIG. 4.

### Example 5—Rilpivirine Particle Size Under Different Storage Conditions

This example explores the effect that freeze drying aqueous compositions comprising rilpivirine particles in suspension has on the particle size of rilpivirine in the corresponding reconstituted aqueous compositions. The freeze dried compositions were reconstituted instantly, i.e. at time t=0, and after storage for 1 month, and after storage for 6 months under various conditions.

Aqueous compositions of rilpivirine nanoparticles (having Dv10 of about 96.5 nm, Dv50 of about 224 nm, and Dv90 of about 509 nm) with the following excipients were prepared:
Rilpivirine (300 mg/mL)
rHuPH20 (2000 U/mL)
Sodium phosphate monohydrate (2 mg/mL)
Citric acid monohydrate (1 mg/mL)
Poloxamer 338 (50 mg/mL)
Sodium hydroxide (q.s. for pH 6)
Water for injection (q.s. ad 1 mL)

Additional component(s) as shown in Table 6 were added, and the pH was adjusted to 6.0 using sodium hydroxide.

TABLE 6
Additional components

| Composition | Additional component 1 | Additional component 2 |
|---|---|---|
| 64 | Sucrose (100 mg/mL) | Glycine (1 mg/mL) |
| 65 | Sucrose (100 mg/mL) | Sodium CMC (10 mg/mL) |
| 66 | Mannitol (50 mg/mL) | Glycine (1 mg/mL) |
| 67 | Mannitol (50 mg/mL) | Sodium CMC (10 mg/mL) |
| 68 | Arginine HCl (50 mg/mL) | — |
| 69 | Sucrose (100 mg/mL) | Arginine HCl (5 mg/mL) |
| 70 | Mannitol (50 mg/mL) | Arginine HCl (5 mg/mL) |

The compositions were freeze dried and reconstituted with water to provide 9 mL of the reconstituted aqueous composition at time t=0, freeze dried and reconstituted with water to provide 9 mL of the reconstituted aqueous composition after being stored under conditions of 25° C./60% RH and 40° C./75% RH for 1 month, and freeze dried and reconstituted with water to provide 9 mL of the reconstituted aqueous composition after being stored under conditions of 25° C./60% RH for 6 months. The rilpivirine particle sizes were measured in both instances following the method below.

The volume-based particle size distribution of the rilpivirine suspensions was determined by means of wet dispersion laser diffraction, using a Malvern Mastersizer 3000 laser diffraction (Malvern Instruments) and Hydro MV wet dispersion module.

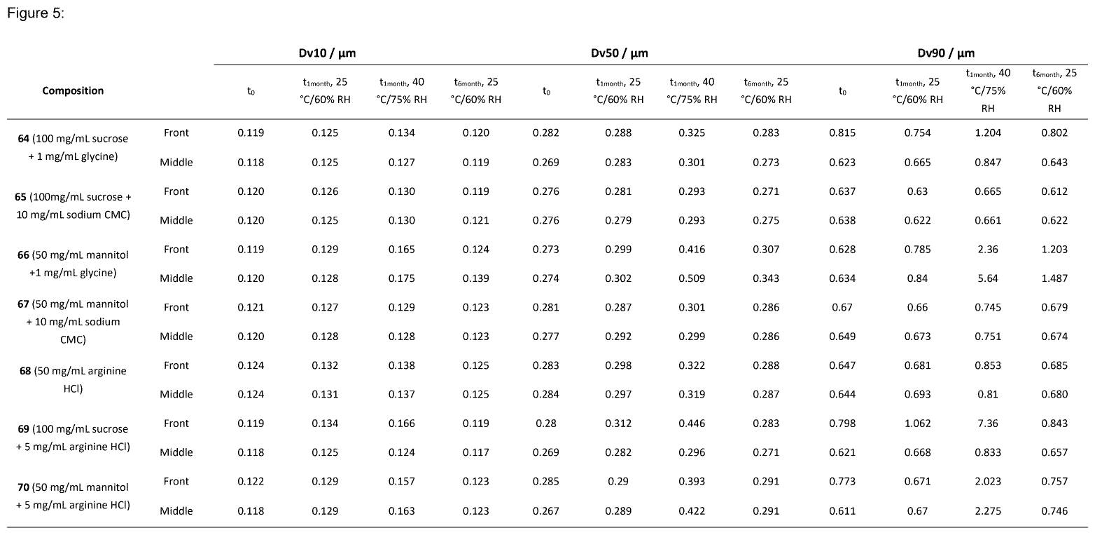

The results are shown in FIG. 5.

At time t=0, i.e. following freeze drying and reconstituting straight away, the particle size was preserved in all compositions.

### Example 6—Freeze Drying and Enzyme Stability Studies Under Different Storage Conditions

This example explores the effect that freeze drying aqueous compositions comprising rHuPH20 and rilpivirine has on enzyme stability in the corresponding reconstituted aqueous compositions. The freeze dried compositions were reconstituted instantly, i.e. at time t=0, after storage for 1 month, and after storage for 6 months under various conditions.

Aqueous compositions 64-70, as described in Example 5 above, were prepared. These compositions were freeze dried and reconstituted with water to provide 9 mL of the reconstituted aqueous composition after being stored under conditions of 25° C./60% RH for 1 month and 6 months.

Enzyme stability was evaluated through one or more of the following: (i) activity (bioassay method); (ii) quantification/aggregation (size-exclusion chromatography/SEC); and (iii) purity (reversed-phase liquid chromatography/RP-LC).

SEC is a size exclusion chromatographic separation method whereby a column is used to distribute the molecules within the solution flowing through according to their broad size range. Monomers, aggregates and fragments elute at different times from the column and hence their relative proportions in a sample can be quantified using a standard UV detector.

The results are shown in Table 8.

TABLE 8
Enzyme stability

| | | Enzyme | | | | |
|---|---|---|---|---|---|---|
| | | quantification/ | | | | |
| | | aggregation | RP-LC Ox2* | | RP-LC Ox1* | |
| | Enzyme | (SEC) (μg/mL) | (Area %) | | (Area %) | |
| | activity | % difference of | t = 1 | t = 6 | t = 1 | t = 6 |
| | % loss after 6 | the t6M @25° C. | month | months | month | months |
| Composition | months @25° C. | and t1M @5° C. | at 25° C. | at 25° C. | at 25° C. | at 25° C. |
| 64 | 35 | −15.1 | 1.9 | 3.7 | 30.5 | 35.1 |
| 65 | — | 0 | 1.5 | 2.1 | 20.4 | 25.7 |
| 66 | 56 | 15.8 | 4.0 | 5.6 | 36.3 | 36.8 |
| 67 | — | −31.0 | 0.6 | 1.8 | 20.4 | 24.2 |
| 68 | 15 | 3.1 | 0.5 | 2.4 | 22.7 | 29.0 |
| 69 | 21 | −20.9 | 1.2 | 3.0 | 27.5 | 34.8 |
| 70 | 49 | −4.2 | 4.2 | 7.6 | 37.7 | 40.6 |

*Ox1 and Ox2 are oxidised forms of rHuPH20. Ox1 retains more enzymatic activity than Ox2. Ox1 and Ox2 are less active than rHuPH20.

The appearance of all compositions was acceptable. The freeze dried compositions all had a relatively low water content (<1% as determined by Karl Fisher). IR analysis confirmed that the samples were homogenous. Identical IR spectra were obtained for samples from the top, middle, and bottom of the vials of freeze dried compositions 64-70.

### Example 7—Freeze Drying and Collapse Temperature Studies Under Different Storage Conditions

This example explores the effect that freeze drying aqueous compositions comprising rHuPH20 and rilpivirine has on collapse temperature (T_{c}).

Aqueous compositions 64-70, as described in Example 5 above, were prepared. The compositions were then freeze dried. The collapse temperature was determined.

The results are shown in Table 9.

TABLE 9
Collapse temperatures (T_{c})

Approximate onset of T_{c}/° C.

| Composition | Fast ramp | Medium ramp with annealing |
|---|---|---|
| 64 | −25.7 | — |
| 65 | −16.0 | — |
| 66 | — | −4.7 |
| 67 | — | −7.2 |
| 68 | −24.3 | — |
| 69 | −14.5 | — |
| 70 | — | −7.9 |

“—” means not tested

### Example 8—Freeze Drying, Enzyme Stability, and Rilpivirine Particle Size Studies Under Different Storage Conditions

This example explores the effect that freeze drying aqueous compositions comprising rHuPH20 and rilpivirine has on collapse temperature, enzyme stability, viscosity and injection force in the corresponding reconstituted aqueous compositions. The freeze dried compositions were reconstituted instantly, i.e. at time t=0, after storage for 2 weeks, 1 month, and 3 months under various conditions.

Aqueous compositions of either (i) rilpivirine nanoparticles (having Dv10 of about 75-200 nm, Dv50 of about 200-500 nm, and Dv90 of about 500-1600 nm, i.e. “nano”) or (ii) rilpivirine microparticles (having Dv10 of about 0.2-0.6 μm, Dv50 of about 1.0-2.5 μm and Dv90 of about 4.0-6.5 μm, i.e. “micro”) with the following excipients were prepared:
Rilpivirine (300 mg/mL)
rHuPH20 (2000 U/mL)
Sodium phosphate monohydrate (2 mg/mL)
Citric acid monohydrate (1 mg/mL)
Poloxamer 338 (see Table 10)
Sodium hydroxide (q.s. for pH 6.0)
Water for injection (q.s. ad 1 mL)

Additional component(s) as shown in Table 10 were added, and the pH was adjusted to 6.0 using sodium hydroxide.

TABLE 10
Compositions and T_{c}

| | Rilpivirine | Poloxamer | Additional | Additional | |
|---|---|---|---|---|---|
| | particle | 338/ | component | component | T_{c}/ |
| Composition | size | mg/mL | 1 | 2 | ° C. |
| 68 | Nano | 50 | Arginine | — | — |
| | | | HCl (50 | | |
| | | | mg/mL) | | |
| 71 | Nano | 50 | Arginine | — | −17.0 |
| | | | HCl (35 | | |
| | | | mg/mL) | | |
| 72 | Nano | 20 | Arginine | Sodium | — |
| | | | HCl (35 | CMC (1 | |
| | | | mg/mL) | mg/mL) | |
| 73 | Micro | 10 | Arginine | Sodium | −26.6 |
| | | | HCl (35 | CMC (1 | |
| | | | mg/mL) | mg/mL) | |
| 74 | Nano | 50 | Arginine | Sodium | — |
| | | | HCl (35 | CMC (3 | |
| | | | mg/mL) | mg/mL) | |
| 75 | Micro | 50 | Arginine | Sodium | — |
| | | | HCl (35 | CMC (3 | |
| | | | mg/mL) | mg/mL) | |
| 76 | Nano | 20 | Arginine | Sodium | — |
| | | | HCl (30 | CMC (3 | |
| | | | mg/mL) | mg/mL) | |
| 77 | Micro | 10 | Arginine | Sodium | −26.8 |
| | | | HCl (30 | CMC (3 | |
| | | | mg/mL) | mg/mL) | |
| 65 | Nano | 50 | Sucrose | Sodium | — |
| | | | (100 | CMC (10 | |
| | | | mg/mL) | mg/mL) | |
| 78 | Nano | 50 | Sucrose | Sodium | −29.5 |
| | | | (100 | CMC (3 | |
| | | | mg/mL) | mg/mL) | |
| 79 | Micro | 50 | Sucrose | Sodium | −23.0 |
| | | | (100 | CMC (3 | |
| | | | mg/mL) | mg/mL) | |
| 80 | Nano | 20 | Sucrose | Sodium | −30.6 |
| | | | (100 | CMC (3 | |
| | | | mg/mL) | mg/mL) | |
| 81 | Micro | 10 | Sucrose | Sodium | −27.0 |
| | | | (100 | CMC (3 | |
| | | | mg/mL) | mg/mL) | |
| 82 | Nano | 20 | Sucrose | Sodium | −27.0 |
| | | | (100 | CMC (1 | |
| | | | mg/mL) | mg/mL) | |
| 83 | Micro | 10 | Sucrose | Sodium | −27.0 |
| | | | (100 | CMC (1 | |
| | | | mg/mL) | mg/mL) | |
| 84 | Nano | 50 | Arginine | Sodium | −19.0 |
| | | | HCl (30 | CMC (3 | |
| | | | mg/mL) | mg/mL) | |
| 85 | Micro | 50 | Arginine | Sodium | −20.4 |
| | | | HCl (30 | CMC (3 | |
| | | | mg/mL) | mg/mL) | |

“—” in T_{c} column means not tested

The compositions were freeze dried and the collapse temperature was determined. The results are shown in Table 10.

The freeze dried compositions all had a relatively low water content (<1% as determined by Karl Fisher). The appearance of all compositions was acceptable.

The compositions were then reconstituted with water to provide 9 mL of the reconstituted aqueous composition after being stored under the following conditions:
2 weeks: 50° C./75% RH
1 month: 30° C./75% RH and 40° C./75% RH
3 months: 30° C./75% RH and 40° C./75% RH

Enzyme stability was evaluated through one or more of the following: (i) activity (bioassay method); (ii) quantification/aggregation (size-exclusion chromatography/SEC) and (iii) purity (reversed-phase liquid chromatography/RP-LC).

The rilpivirine particle sizes were measured as follows. The volume-based particle size distribution of the rilpivirine suspensions was determined by means of wet dispersion laser diffraction, using a Malvern Mastersizer 3000 laser diffraction (Malvern Instruments) and Hydro MV wet dispersion module.

The viscosity of the compositions was measured using a HAAKE Mars 60 rheometer. 1.5 mL of the aqueous reconstituted composition was withdrawn from the vial using a 2 mL syringe equipped with an 18G needle. The sample was added dropwise to the plate (no air bubbles were present). The shear rate sweep was performed from 0.1 till 1000 sec-1 with a cone 60/1° and plate.

The injection force was measured using a 20 mL Luer Lock syringe equipped with 23 G×¾ inch winged infusion set.

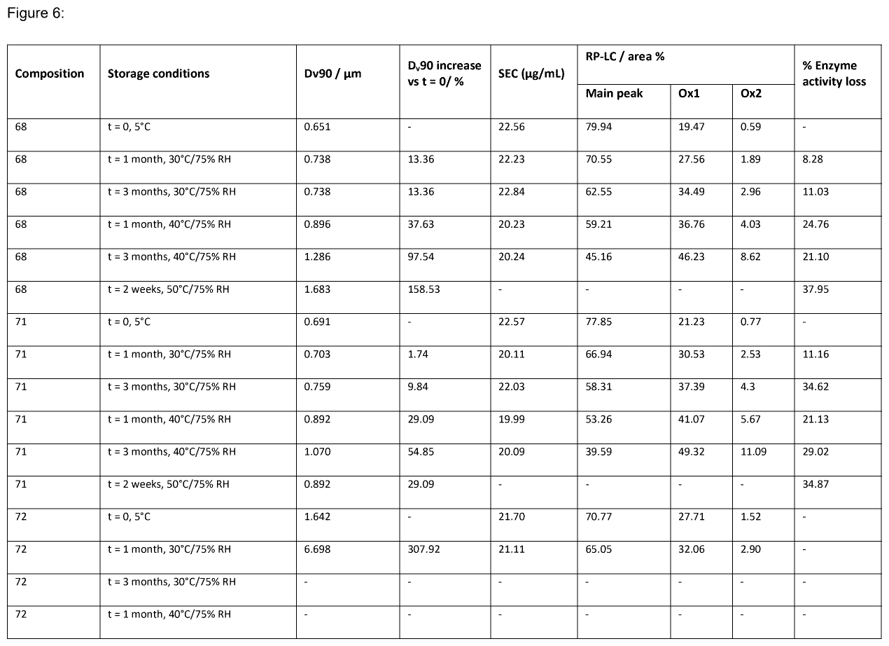

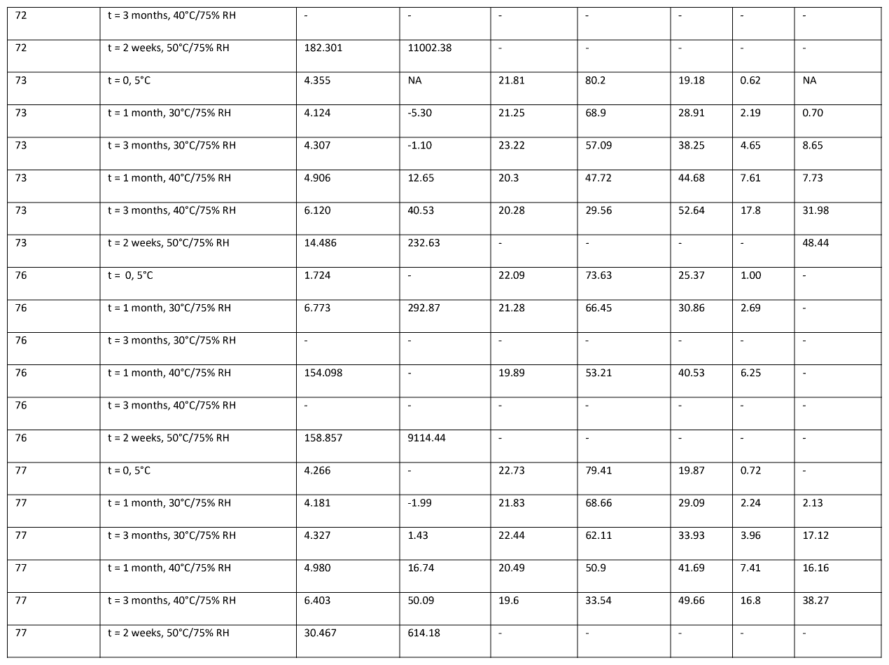

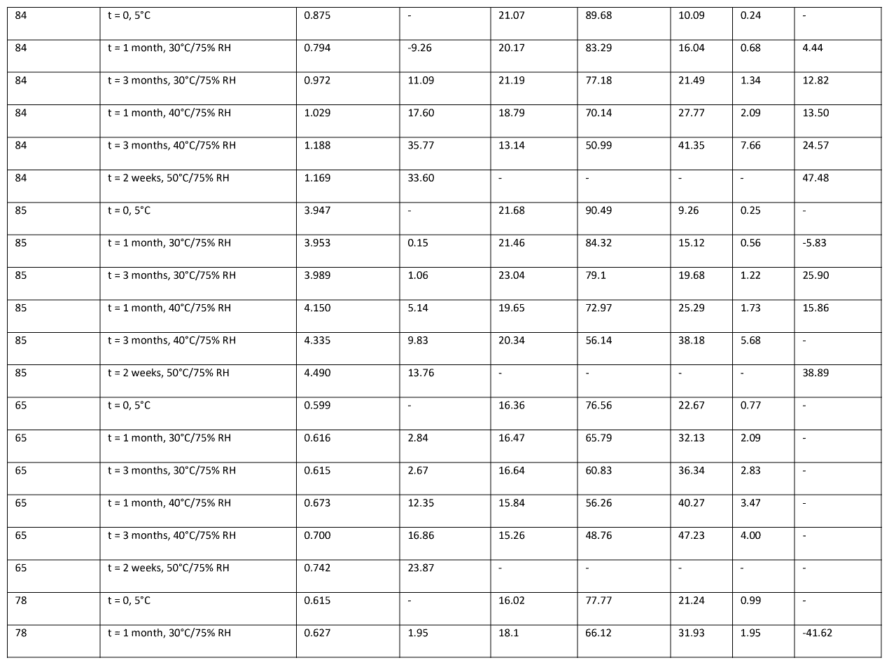

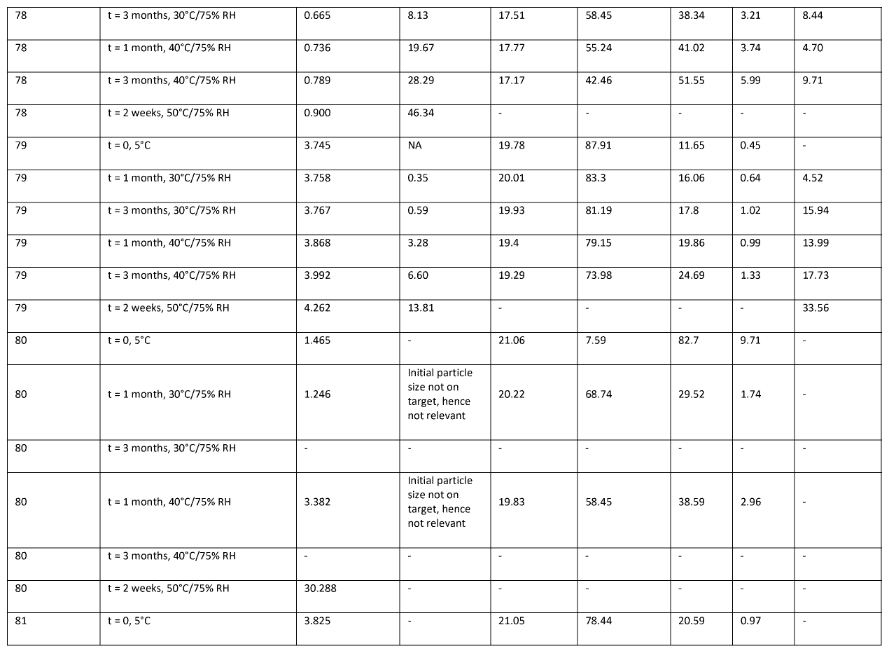

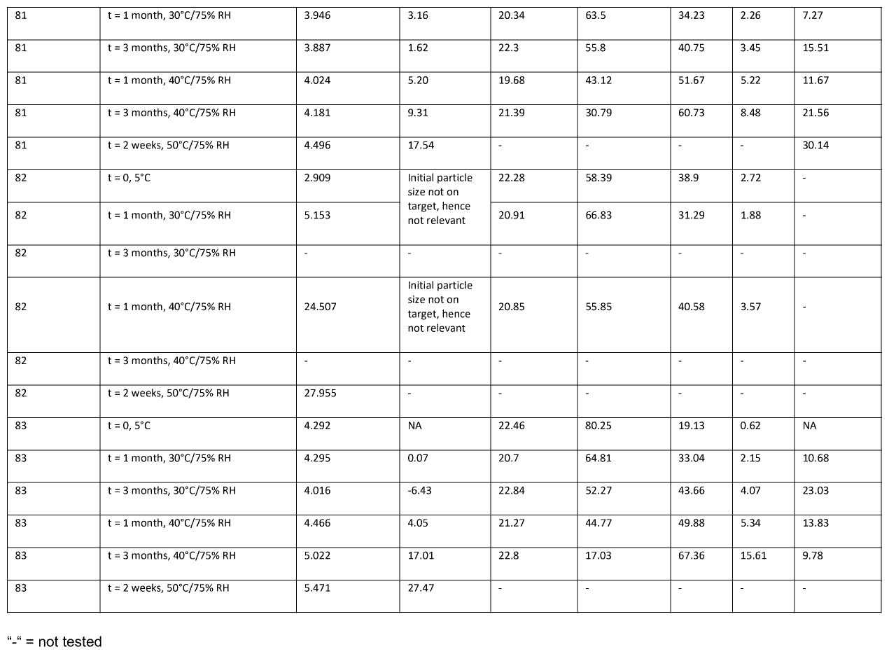

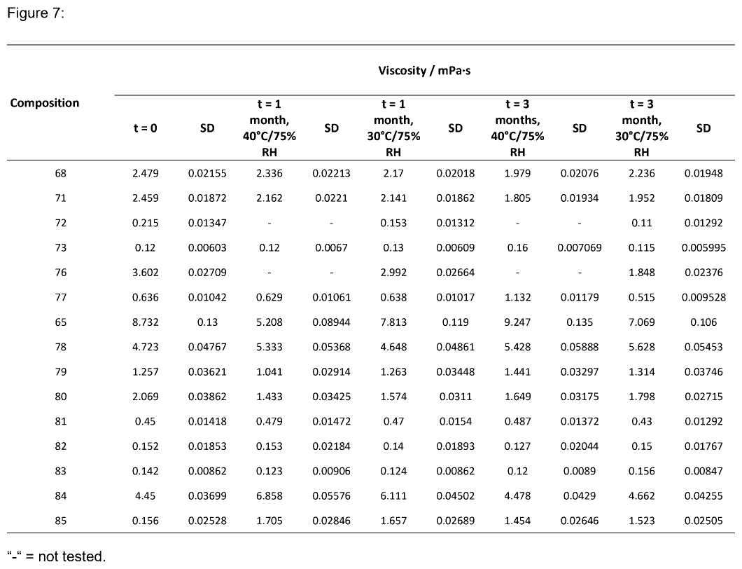

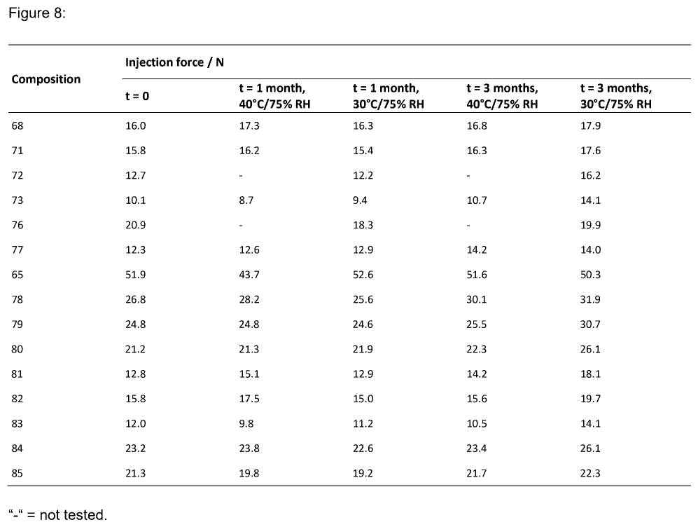

The results are shown in FIGS. 6, 7, and 8 (nb. Ox1 and Ox2 are oxidised forms of rHuPH20. Ox1 retains more enzymatic activity than Ox2. Ox1 and Ox2 are less active than rHuPH20).

### Example 9—Enzyme Stability Studies Under Different Storage Conditions

This example compares the fluorescence emission spectra of rHuPH20 compositions after storage under different conditions.

Aqueous compositions 45-57, as described in Example 3 above, were prepared. The spectra of the compositions were collected immediately after storage at 5° C. and after storage at 50° C. for one day. In addition to peak maximum and minimum, the intensity ratio 350/330 nm was calculated at each time point and plotted as a function of storage time. The slope of this curve was used to have a semi-quantitative thermal stability assessment and comparison among compositions.

### Composition Preparation

200 μl of each composition was dispensed manually into an UV-STAR®, COC, 96-well flat-bottom microplate.

The fluorescence emission spectrum of the samples was measured before and after stressing via a Tecan plate reader (Infinite 200). The following method parameters were used:

### Temperature:

Parameter (on), temperature=25.0° C.

### Wait for Temperature:

Parameter minimum=24.5° C.; maximum=25.5° C.

### Fluorescence Intensity Scan:

Scan selection (emission scan)
Mode (top)
Excitation wavelengths: from =280 nm; bandwidth: 230 . . . 315: 5 nm; 316 . . . 850: 9 nm
Emission wavelengths: from =310 nm; to =420 nm; step=1 nm; bandwidth: 280 . . . 850: 20 nm; 111 measurements
Integration: lag time=0 μs; integration time=20 μs
Read: number of flashes=25; settle time=0 ms
Gain (manual=95)
Label: name=Label1

The results are shown in Table 11, below.

TABLE 11
Slope of the ratio between 350 nm/330 nm versus time

| | | | | | | | Difference t₁ − t₀ | | |
|---|---|---|---|---|---|---|---|---|---|
| | t₀ | | | t₁ = day 1 at 50° C. | | | Δ330 | Δ350 | Δ (Ratio |
| Composition | 330 nm | 350 nm | 350/330 | 330 nm | 350 nm | 350/330 | nm | nm | 350/330) |
| 52 | 9126 | 6871 | 0.753 | 6091 | 4993 | 0.820 | 3035 | 1878 | 0.067 |
| 48 | 6036 | 4524 | 0.750 | 4084 | 3345 | 0.819 | 1952 | 1179 | 0.070 |
| 49 | 7139 | 5365 | 0.752 | 4927 | 3953 | 0.802 | 2212 | 1412 | 0.051 |
| 55 | 4983 | 3748 | 0.752 | 3563 | 2895 | 0.813 | 1420 | 853 | 0.060 |
| 47 | 5545 | 4226 | 0.762 | 4412 | 3565 | 0.808 | 1133 | 661 | 0.046 |
| 46 | 4997 | 3834 | 0.767 | 4134 | 3343 | 0.809 | 863 | 491 | 0.041 |
| 45 | 5314 | 4045 | 0.761 | 4333 | 3548 | 0.819 | 981 | 497 | 0.058 |
| 50 | 4615 | 3586 | 0.777 | 3883 | 3147 | 0.810 | 732 | 439 | 0.033 |
| 51 | 5175 | 3942 | 0.762 | 4334 | 3502 | 0.808 | 841 | 440 | 0.046 |
| 53 | 6991 | 5403 | 0.773 | 5344 | 4467 | 0.836 | 1647 | 936 | 0.063 |
| 54 | 7458 | 5662 | 0.759 | 5654 | 4589 | 0.812 | 1804 | 1073 | 0.052 |
| 56 | 4276 | 3330 | 0.779 | 3262 | 2728 | 0.836 | 1014 | 602 | 0.058 |
| 57 | 3727 | 2944 | 0.790 | 2947 | 2494 | 0.846 | 780 | 450 | 0.056 |

### Example 10—Melting Temperature Studies Under Different Storage Conditions

This example compares the melting temperatures of rHuPH20 in various compositions under different storage conditions.

Aqueous compositions of rilpivirine nanoparticles (having Dv10 of 75-200 nm, Dv50 of 200-500 nm, and Dv90 of 500-1600 nm) with the following excipients were prepared:
rHuPH20 (2000 U/mL)
Sodium dihydrogen phosphate monohydrate (2 mg/mL)
Citric acid monohydrate (1 mg/mL)
Poloxamer 338 (50 mg/mL)
Sodium hydroxide (q.s. for pH 6)
Water for injection (q.s. ad 1 mL)

Compositions 86-171 were then prepared by adding the component(s) as shown in Table 12, and the pH was adjusted to 6.0 by varying the amount of sodium hydroxide in the composition.

TABLE 12
Tₘ under different storage conditions

| | | | | | | Tₘ (t = 1 |
|---|---|---|---|---|---|---|
| | | | | | Tₘ (t = 1 | week at |
| | | | | Tₘ (t = 0) | week at | 40° C.) |
| | Additional | Additional | Tₘ (t = 0)/ | SD (n = | 40° C.)/ | SD (n = |
| Composition | component 1 | component 2 | ° C. | 3)/° C. | ° C. | 3)/° C. |
| 86 | Sucrose (100 | Glycine (1 | 48.52 | 0.05 | N/A** | N/A** |
| | mg/mL) | mg/mL) | | | | |
| 87 | Sucrose (100 | Glycine (3 | 48.61 | 0.03 | N/A** | N/A** |
| | mg/mL) | mg/mL) | | | | |
| 88 | Sucrose (100 | Glycine (5 | 48.64 | 0.01 | N/A** | N/A** |
| | mg/mL) | mg/mL) | | | | |
| 89 | Sucrose (100 | Glycine (7 | 48.75 | 0.07 | N/A** | N/A** |
| | mg/mL) | mg/mL) | | | | |
| 90 | Sucrose (100 | Glycine (9 | 48.98 | 0.1 | N/A** | N/A** |
| | mg/mL) | mg/mL) | | | | |
| 91 | Sucrose (100 | Glycine (11 | 48.93 | 0.1 | N/A** | N/A** |
| | mg/mL) | mg/mL) | | | | |
| 92 | Sucrose (100 | Glycine (13 | 49.14 | 0.1 | N/A** | N/A** |
| | mg/mL) | mg/mL) | | | | |
| 93 | Sucrose (100 | Glycine (15 | 49.25 | 0.07 | N/A** | N/A** |
| | mg/mL) | mg/mL) | | | | |
| 94 | Sucrose (100 | Glycine (17 | 49.16 | 0.05 | N/A** | N/A** |
| | mg/mL) | mg/mL) | | | | |
| 95 | Sucrose (100 | Glycine (19 | 49.45 | 0.05 | N/A** | N/A** |
| | mg/mL) | mg/mL) | | | | |
| 96 | Sucrose (100 | Glycine (21 | 49.44 | 0.06 | N/A** | N/A** |
| | mg/mL) | mg/mL) | | | | |
| 97 | Sucrose (100 | Glycine (23 | 49.71 | 0.08 | N/A** | N/A** |
| | mg/mL) | mg/mL) | | | | |
| 98 | Sucrose (100 | Glycine (25 | 49.54 | 0.15 | N/A** | N/A** |
| | mg/mL) | mg/mL) | | | | |
| 99 | Mannitol (50 | Glycine (1 | 48.16 | 0.03 | N/A** | N/A** |
| | mg/mL) | mg/mL) | | | | |
| 100 | Mannitol (50 | Glycine (3 | 47.95 | 0.11 | N/A** | N/A** |
| | mg/mL) | mg/mL) | | | | |
| 101 | Mannitol (50 | Glycine (5 | 48.12 | 0.05 | N/A** | N/A** |
| | mg/mL) | mg/mL) | | | | |
| 102 | Mannitol (50 | Glycine (7 | 48.41 | 0.38 | N/A** | N/A** |
| | mg/mL) | mg/mL) | | | | |
| 103 | Mannitol (50 | Glycine (9 | 48.56 | 0.03 | N/A** | N/A** |
| | mg/mL) | mg/mL) | | | | |
| 104 | Mannitol (50 | Glycine (11 | 48.28 | 0.04 | N/A** | N/A** |
| | mg/mL) | mg/mL) | | | | |
| 105 | Mannitol (50 | Glycine (13 | 48.37 | 0.08 | N/A** | N/A** |
| | mg/mL) | mg/mL) | | | | |
| 106 | Mannitol (50 | Glycine (15 | 48.45 | 0.06 | N/A** | N/A** |
| | mg/mL) | mg/mL) | | | | |
| 107 | Mannitol (50 | Glycine (17 | 48.42 | 0.04 | N/A** | N/A** |
| | mg/mL) | mg/mL) | | | | |
| 108 | Mannitol (50 | Glycine (19 | 48.69 | 0.07 | N/A** | N/A** |
| | mg/mL) | mg/mL) | | | | |
| 109 | Mannitol (50 | Glycine (21 | 48.83 | 0.02 | N/A** | N/A** |
| | mg/mL) | mg/mL) | | | | |
| 110 | Mannitol (50 | Glycine (23 | 48.91 | 0.02 | N/A** | N/A** |
| | mg/mL) | mg/mL) | | | | |
| 111 | Mannitol (50 | Glycine (25 | 48.93 | 0.09 | N/A** | N/A** |
| | mg/mL) | mg/mL) | | | | |
| 112 | Mannitol (50 | Sodium CMC | 53.93 | 0.04 | 54.51 | 0.23 |
| | mg/mL) | (5 mg/mL) | | | | |
| 113 | Mannitol (50 | Sodium CMC | 53.64 | 0.07 | 54.39 | 0.42 |
| | mg/mL) | (10 mg/mL) | | | | |
| 114 | Mannitol (50 | Sodium CMC | 53.4 | 0.02 | 54.19 | 0.29 |
| | mg/mL) | (15 mg/mL) | | | | |
| 115 | Mannitol (50 | Sodium CMC | N/A* | N/A* | 53.49 | 0.26 |
| | mg/mL) | (20 mg/mL) | | | | |
| 116 | Sucrose (100 | Sodium CMC | 54.71 | 0.01 | 55.28 | 0.37 |
| | mg/mL) | (5 mg/mL) | | | | |
| 117 | Sucrose (100 | Sodium CMC | 54.25 | 0.04 | 54.94 | 0.31 |
| | mg/mL) | (10 mg/mL) | | | | |
| 118 | Sucrose (100 | Sodium CMC | 54.01 | 0.09 | 54.52 | 0.4 |
| | mg/mL) | (15 mg/mL) | | | | |
| 119 | Mannitol (50 | Arginine (5 | 41.14 | 0.05 | N/A** | N/A** |
| | mg/mL) | mg/mL) | | | | |
| 120 | Mannitol (50 | Arginine (10 | 40.3 | 0.07 | N/A** | N/A** |
| | mg/mL) | mg/mL) | | | | |
| 121 | Mannitol (50 | Arginine (15 | 39.84 | 0.09 | N/A** | N/A** |
| | mg/mL) | mg/mL) | | | | |
| 122 | Mannitol (50 | Arginine (20 | 39.51 | 0.09 | N/A** | N/A** |
| | mg/mL) | mg/mL) | | | | |
| 123 | Mannitol (50 | Arginine (25 | 39.69 | 0.03 | N/A** | N/A** |
| | mg/mL) | mg/mL) | | | | |
| 124 | Sucrose (100 | Arginine (5 | 41.61 | 0.13 | N/A** | N/A** |
| | mg/mL) | mg/mL) | | | | |
| 125 | Sucrose (100 | Arginine (10 | 40.97 | 0.12 | N/A** | N/A** |
| | mg/mL) | mg/mL) | | | | |
| 126 | Sucrose (100 | Arginine (15 | 40.53 | 0.06 | N/A** | N/A** |
| | mg/mL) | mg/mL) | | | | |
| 127 | Sucrose (100 | Arginine (20 | 40.38 | 0.04 | N/A** | N/A** |
| | mg/mL) | mg/mL) | | | | |
| 128 | Sucrose (100 | Arginine (25 | 40.15 | 0.1 | N/A** | N/A** |
| | mg/mL) | mg/mL) | | | | |
| 129 | Glucose (50 | — | 47.41 | 0.04 | N/A** | N/A** |
| | mg/mL) | | | | | |
| 130 | Sucrose (100 | — | 48.46 | 0.15 | N/A** | N/A** |
| | mg/mL) | | | | | |
| 131 | Glucose (50 | Sodium CMC | 53.82 | 0.02 | 54.41 | 0.1 |
| | mg/mL) | (5 mg/mL) | | | | |
| 132 | Glucose (50 | Sodium CMC | 53.57 | 0.06 | 54.24 | 0.08 |
| | mg/mL) | (10 mg/mL) | | | | |
| 133 | Glucose (50 | Sodium CMC | 53.27 | 0.23 | 53.88 | 0.13 |
| | mg/mL) | (15 mg/mL) | | | | |
| 134 | Glucose (50 | Glycine (5 | 48.03 | 0.11 | N/A** | N/A** |
| | mg/mL) | mg/mL) | | | | |
| 135 | Glucose (50 | Glycine (10 | 47.78 | 0.16 | N/A** | N/A** |
| | mg/mL) | mg/mL) | | | | |
| 136 | Glucose (50 | Glycine (15 | 48.81 | 0.14 | N/A** | N/A** |
| | mg/mL) | mg/mL) | | | | |
| 137 | Glucose (50 | Glycine (20 | 48.17 | 0.07 | N/A** | N/A** |
| | mg/mL) | mg/mL) | | | | |
| 138 | Glucose (50 | Glycine (25 | 48.3 | 0.19 | N/A** | N/A** |
| | mg/mL) | mg/mL) | | | | |
| 139 | Glucose (50 | Arginine (5 | 41.61 | 0.1 | N/A** | N/A** |
| | mg/mL) | mg/mL) | | | | |
| 140 | Glucose (50 | Arginine (10 | 41.33 | 0.1 | N/A** | N/A** |
| | mg/mL) | mg/mL) | | | | |
| 141 | Glucose (50 | Arginine (15 | 41.29 | 0.04 | N/A** | N/A** |
| | mg/mL) | mg/mL) | | | | |
| 142 | Glucose (50 | Arginine (20 | 41.1 | 0.09 | N/A** | N/A** |
| | mg/mL) | mg/mL) | | | | |
| 143 | Glucose (50 | Arginine (25 | 41 | 0.13 | N/A** | N/A** |
| | mg/mL) | mg/mL) | | | | |
| 144 | Glucose (50 | Histidine (5 | 46.72 | 0.06 | N/A** | N/A** |
| | mg/mL) | mg/mL) | | | | |
| 145 | Glucose (50 | Histidine (10 | 46.18 | 0.03 | N/A** | N/A** |
| | mg/mL) | mg/mL) | | | | |
| 146 | Glucose (50 | Histidine (15 | 46.15 | 0.05 | N/A** | N/A** |
| | mg/mL) | mg/mL) | | | | |
| 147 | Glucose (50 | Histidine (20 | 45.57 | 0.08 | N/A** | N/A** |
| | mg/mL) | mg/mL) | | | | |
| 148 | Glucose (50 | Histidine (25 | 45.48 | 0.03 | N/A** | N/A** |
| | mg/mL) | mg/mL) | | | | |
| 149 | — | Glycine (1 | 46.94 | 0.07 | N/A** | N/A** |
| | | mg/mL) | | | | |
| 150 | — | Glycine (3 | 46.91 | 0.08 | N/A** | N/A** |
| | | mg/mL) | | | | |
| 151 | — | Glycine (5 | 46.99 | 0.09 | N/A** | N/A** |
| | | mg/mL) | | | | |
| 152 | — | Glycine (7 | 47.17 | 0.09 | N/A** | N/A** |
| | | mg/mL) | | | | |
| 153 | — | Glycine (9 | 47.2 | 0.09 | N/A** | N/A** |
| | | mg/mL) | | | | |
| 154 | — | Glycine (11 | 47.18 | 0.05 | N/A** | N/A** |
| | | mg/mL) | | | | |
| 155 | — | Glycine (13 | 47.72 | 0.11 | N/A** | N/A** |
| | | mg/mL) | | | | |
| 156 | — | Glycine (15 | 47.28 | 0.03 | N/A** | N/A** |
| | | mg/mL) | | | | |
| 157 | — | Glycine (17 | 47.34 | 0.11 | N/A** | N/A** |
| | | mg/mL) | | | | |
| 158 | — | Glycine (19 | 47.58 | 0.09 | N/A** | N/A** |
| | | mg/mL) | | | | |
| 159 | — | Glycine (21 | 47.41 | 0.04 | N/A** | N/A** |
| | | mg/mL) | | | | |
| 160 | — | Glycine (23 | 47.52 | 0.07 | N/A** | N/A** |
| | | mg/mL) | | | | |
| 161 | — | Glycine (25 | 47.61 | 0.14 | N/A** | N/A** |
| | | mg/mL) | | | | |
| 162 | — | Sodium CMC | 53.87 | 0.06 | 54.2 | 0.38 |
| | | (1 mg/mL) | | | | |
| 163 | — | Sodium CMC | 53.34 | 0.03 | 53.96 | 0.35 |
| | | (3 mg/mL) | | | | |
| 164 | — | Sodium CMC | 53.15 | 0.01 | 53.58 | 0.06 |
| | | (5 mg/mL) | | | | |
| 165 | — | Sodium CMC | 53.22 | 0.04 | 53.64 | 0.3 |
| | | (7 mg/mL) | | | | |
| 166 | — | Sodium CMC | 53 | 0.06 | 53.22 | 0.07 |
| | | (9 mg/mL) | | | | |
| 167 | — | Sodium CMC | 52.84 | 0.06 | 53.04 | 0.07 |
| | | (11 mg/mL) | | | | |
| 168 | — | Sodium CMC | 52.85 | 0.08 | 52.93 | 0.11 |
| | | (13 mg/mL) | | | | |
| 169 | — | Sodium CMC | 52.45 | 0.05 | 52.76 | 0.18 |
| | | (15 mg/mL) | | | | |
| 170 | — | Sodium CMC | 52.31 | 0.02 | 52.64 | 0.07 |
| | | (17 mg/mL) | | | | |
| 171 | — | Sodium CMC | N/A* | N/A* | 52.61 | 0.09 |
| | | (19 mg/mL) | | | | |

N/A* = Note that for high sodium CMC concentrations (>17 mg/mL) the capillary chips of the nanoDSF instrument could not be loaded, presumably due to high solution viscosity.
N/A** = Note that for these conditions either the enzyme was denatured due to the applied temperature stress, or the capillaries could not be loaded to high viscosity

Compositions 86-171 were prepared as set out above and then stored in a refrigerator at 5° C. for between 2 days and 2 weeks. The Tₘ values were measured straight away (i.e. “t=0”) and after storage for 1 week at 40° C. using the method as described in Example 1.

The results are shown in Table 12.

Also described herein are the following numbered clauses.

1. A solid composition obtainable by freeze drying an aqueous composition comprising rilpivirine or a pharmaceutically acceptable salt thereof.

2. The solid composition according to clause 1, wherein the aqueous composition additionally comprises a hyaluronidase.

3. The solid composition according to clause 2, wherein the hyaluronidase is recombinant human hyaluronidase

4. The solid composition according to clause 3, wherein the recombinant human hyaluronidase is rHuPH20.

5. The solid composition according to any one of clauses 2-4, wherein the aqueous composition comprises from about 1,500 U/mL to about 2,500 U/mL of the hyaluronidase.

6. The solid composition according to clause 5, wherein the aqueous composition comprises from about 1,800 U/mL to about 2,200 U/mL of the hyaluronidase.

7. The solid composition according to clause 6, wherein the aqueous composition comprises about 2,000 U/mL of the hyaluronidase.

8. The solid composition according to any one of the preceding clauses, wherein the aqueous composition comprises rilpivirine or a pharmaceutically acceptable salt thereof in the form of particles suspended in the aqueous composition.

9. The solid composition according to clause 8, wherein the aqueous composition comprises rilpivirine or a pharmaceutically acceptable salt thereof in the form of micro- or nanoparticles suspended in the aqueous composition.

10. The solid composition according to clause 8 or 9, wherein the particles have a Dv90 of from about 100 nm to about 10 μm.

11. The solid composition according to clause 10, wherein the particles have a Dv90 of from about 500 nm to about 6 μm, optionally wherein the particles have a Dv90 of from about 500 nm to about 1,600 nm.

12. The solid composition according to clause 10, wherein the particles have a Dv90 of about 500 nm to about 700 nm.

13. The solid composition according to any one of clauses 8-12, wherein the particles have a Dv10 of from about 75 nm to about 200 nm.

14. The solid composition according to any one of clauses 8-13, wherein the particles have a Dv50 of from about 200 nm to about 500 nm.

15. The solid composition according to any one of clauses 8-10, wherein the particles have a Dv90 of from about 4 μm to about 6 μm.

16. The solid composition according to clause 15, wherein the particles have a Dv90 of about 5 μm to about 6 μm.

17. The solid composition according to any one of clauses 8-10 or 15 or 16, wherein the particles have a Dv10 of from about 300 nm to about 500 nm, and/or wherein the particles have a Dv50 of from about 1.5 μm to about 2 μm.

18. The solid composition according to any one of the preceding clauses, wherein the aqueous composition comprises from about 200 mg/mL to about 400 mg/mL rilpivirine or a pharmaceutically acceptable salt thereof.

19. The solid composition according to clause 18, wherein the aqueous composition comprises from about 250 mg/mL to about 350 mg/mL rilpivirine or a pharmaceutically acceptable salt thereof.

20. The solid composition according to clause 19, wherein the aqueous composition comprises about 300 mg/mL rilpivirine or a pharmaceutically acceptable salt thereof.

21. The solid composition according to any one of the preceding clauses, wherein the aqueous composition comprises rilpivirine, i.e. rilpivirine in free base form.

22. The solid composition according to any one of the preceding clauses, wherein the aqueous composition additionally comprises carboxymethyl cellulose or a pharmaceutically acceptable salt thereof.

23. The solid composition according to clause 22, wherein the carboxymethyl cellulose or a pharmaceutically acceptable salt thereof is sodium carboxymethyl cellulose.

24. The solid composition according to clause 22 or 23, wherein the carboxymethyl cellulose or a pharmaceutically acceptable salt thereof is not cross-linked.

25. The solid composition according to any one of clauses 22-24, wherein the carboxymethyl cellulose or a pharmaceutically acceptable salt thereof has a degree of substitution (carboxymethyl to cellulose) of from about 0.5 to about 1.

26. The solid composition according to any one of clauses 22-25, wherein the carboxymethyl cellulose or a pharmaceutically acceptable salt thereof has a molecular weight of from about 90 kDa to about 750 kDa, and/or wherein the carboxymethyl cellulose or a pharmaceutically acceptable salt thereof has a viscosity of from about 30 mPa·s to about 50 mPa·s,

27. The solid composition according to clause 22 or 26, wherein the aqueous composition comprises from about 1 mg/mL to about 100 mg/mL carboxymethyl cellulose or a pharmaceutically acceptable salt thereof.

28. The solid composition according to clause 27, wherein the aqueous composition comprises from about 1 mg/mL to about 50 mg/mL carboxymethyl cellulose or a pharmaceutically acceptable salt thereof.

29. The solid composition according to clause 27, wherein the aqueous composition comprises from about 1 mg/mL to about 5 mg/mL carboxymethyl cellulose or a pharmaceutically acceptable salt thereof.

30. The solid composition according to clause 29, wherein the aqueous composition comprises about 3 mg/mL carboxymethyl cellulose or a pharmaceutically acceptable salt thereof.

31. The solid composition according to clause 27, wherein the aqueous composition comprises from about 10 mg/mL to about 25 mg/mL carboxymethyl cellulose or a pharmaceutically acceptable salt thereof.

32. The solid composition according to clause 22 or 31, wherein the aqueous composition comprises from about 0.05 mg carboxymethyl cellulose or a pharmaceutically acceptable salt thereof per 100 U hyaluronidase to about 2.5 mg carboxymethyl cellulose or a pharmaceutically acceptable salt thereof per 100 U hyaluronidase.

33. The solid composition according to clause 32, wherein the aqueous composition comprises from about 0.5 mg carboxymethyl cellulose or a pharmaceutically acceptable salt thereof per 100 U hyaluronidase to about 2 mg carboxymethyl cellulose or a pharmaceutically acceptable salt thereof per 100 U hyaluronidase.

34. The solid composition according to clause 32, wherein the aqueous composition comprises from about 0.05 mg carboxymethyl cellulose or a pharmaceutically acceptable salt thereof per 100 U hyaluronidase to about 0.25 mg carboxymethyl cellulose or a pharmaceutically acceptable salt thereof per 100 U hyaluronidase.

35. The solid composition according to clause 34, wherein the aqueous composition comprises about 0.15 mg carboxymethyl cellulose or a pharmaceutically acceptable salt thereof per 100 U hyaluronidase.

36. The solid composition according to any one of the preceding clauses, wherein the aqueous composition additionally comprises a cryoprotectant.

37. The solid composition according to clause 36, wherein the cryoprotectant is a sugar, sugar alcohol or an amino acid or a pharmaceutically acceptable salt thereof.

38. The solid composition according to clause 37, wherein the cryoprotectant is a sugar or sugar alcohol.

39. The solid composition according to clause 38, wherein the sugar or sugar alcohol is selected from mannitol and sucrose.

40. The solid composition according to clause 38, wherein the sugar or sugar alcohol is sucrose.

41. The solid composition according to clause 37, wherein the cryoprotectant is an amino acid or a pharmaceutically acceptable salt thereof.

42. The solid composition according to clause 41, wherein the amino acid or a pharmaceutically acceptable salt thereof is selected from arginine, glycine, and histidine, optionally wherein the amino acid or a pharmaceutically acceptable salt thereof is selected from arginine and glycine.

43. The solid composition according to clause 42, wherein the amino acid or a pharmaceutically acceptable salt thereof is arginine, for example arginine HCl.

44. The solid composition according to any one of clauses 36-43, wherein the cryoprotectant is at least 95% crystalline.

45. The solid composition according to any one of clauses 36-45, wherein the aqueous composition comprises from about 1 mg/mL to about 200 mg/mL of the cryoprotectant.

46. The solid composition according to clause 44, wherein the aqueous composition comprises from about 1 mg/mL to about 150 mg/mL of the cryoprotectant.

47. The solid composition according to any one of clauses 37-39, wherein the aqueous composition comprises from about 25 mg/mL to about 125 mg/mL of the sugar or sugar alcohol.

48. The solid composition according to clause 47, wherein the aqueous composition comprises from about 75 mg/mL to about 125 mg/mL of the sugar or sugar alcohol.

49. The solid composition according to clause 48, wherein the aqueous composition comprises about 100 mg/mL of the sugar or sugar alcohol.

50. The solid composition according to clause 47, wherein the aqueous composition comprises from about 50 mg/mL to about 100 mg/mL of the sugar or sugar alcohol.

51. The solid composition according to any one of clauses 37 and 41-43, wherein the aqueous composition comprises from about 1 mg/mL to about 75 mg/mL of the amino acid or a pharmaceutically acceptable salt thereof.

52. The solid composition according to clause 51, wherein the aqueous composition comprises from about 1 mg/mL to about 50 mg/mL of the amino acid or a pharmaceutically acceptable salt thereof.

53. The solid composition according to clause 52, wherein the aqueous composition comprises from about 25 mg/mL to about 35 mg/mL of the amino acid or a pharmaceutically acceptable salt thereof.

54. The solid composition according to clause 53, wherein the aqueous composition comprises about 30 mg/mL of the amino acid or a pharmaceutically acceptable salt thereof.

55. The solid composition according to any one of clauses 36-54, wherein the ratio of rilpivirine or a pharmaceutically acceptable salt thereof to cryoprotectant in the aqueous composition is from about 1:1 (w/w) to about 20:1 (w/w).

56. The solid composition according to clause 55, wherein the ratio of rilpivirine or a pharmaceutically acceptable salt thereof to cryoprotectant in the aqueous composition is from about 2:1 (w/w) to about 10:1 (w/w).

57. The solid composition according to clause 56, wherein the ratio of rilpivirine or a pharmaceutically acceptable salt thereof to cryoprotectant in the aqueous composition is from about 3:1 (w/w) to about 10:1 (w/w).

58. The solid composition according to clause 56, wherein the ratio of rilpivirine or a pharmaceutically acceptable salt thereof to cryoprotectant in the aqueous composition is from about 2:1 (w/w) to about 7:1 (w/w).

59. The solid composition according to clause 58, wherein the ratio of rilpivirine or a pharmaceutically acceptable salt thereof to cryoprotectant in the aqueous composition is from about 3:1 (w/w) to about 6:1 (w/w).

60. The solid composition according to any one of clauses 36-59 wherein the ratio of carboxymethyl cellulose or a pharmaceutically acceptable salt thereof to cryoprotectant is from about 1:1 (w/w) to about 1:100 (w/w), optionally from about 1:1 (w/w) to about 1:50 (w/w).

61. The solid composition according to clause 60, wherein the ratio of carboxymethyl cellulose or a pharmaceutically acceptable salt thereof to cryoprotectant is from about 1:8 (w/w) to about 1:40 (w/w).

62. The solid composition according to any one of clauses 36-59 wherein the ratio of carboxymethyl cellulose or a pharmaceutically acceptable salt thereof to cryoprotectant is from about 1:2 (w/w) to about 1:15 (w/w).

63. The solid composition according to clause 62, wherein the ratio of carboxymethyl cellulose or a pharmaceutically acceptable salt thereof to cryoprotectant is from about 1:4 (w/w) to about 1:11 (w/w).

64. The solid composition according to clause 63, wherein the ratio of carboxymethyl cellulose or a pharmaceutically acceptable salt thereof to cryoprotectant is from about 1:5 (w/w) to about 1:10 (w/w).

65. The solid composition according to any one of clauses 36-59, wherein the ratio of carboxymethyl cellulose or a pharmaceutically acceptable salt thereof to cryoprotectant is from about 1:9 (w/w) to about 1:11 (w/w).

66. The solid composition according to any one of clauses 36-59, wherein the ratio of carboxymethyl cellulose or a pharmaceutically acceptable salt thereof to cryoprotectant is from about 1:30 (w/w) to about 1:35 (w/w).

67. The solid composition according to any one of the preceding clauses, wherein the aqueous composition additionally comprises a poloxamer, for example poloxamer 338.

68. The solid composition according to clause 67, when dependent on clause 8, wherein when the rilpivirine or a pharmaceutically acceptable salt thereof has a Dv90 of less than about 1600 nm the ratio of rilpivirine or a pharmaceutically acceptable salt thereof to poloxamer is from about 3:1 (w/w) to about 15:1 (w/w).

69. The solid composition according to clause 68, wherein when the rilpivirine or a pharmaceutically acceptable salt thereof has a Dv90 of less than about 1600 nm the ratio of rilpivirine or a pharmaceutically acceptable salt thereof to poloxamer is from about 6:1 (w/w) to about 15:1 (w/w).

70. The solid composition according to clause 69, wherein when the rilpivirine or a pharmaceutically acceptable salt thereof has a Dv90 of less than about 1600 nm the ratio of rilpivirine or a pharmaceutically acceptable salt thereof to poloxamer is about 6:1 (w/w).

71. The solid composition according to clause 67, wherein the relative amount (w/w) of rilpivirine or a pharmaceutically acceptable salt thereof to poloxamer is less than about 12:1 (w/w).

72. The solid composition according to any one of the preceding clauses, wherein the aqueous composition additionally comprises a poloxamer, sodium dihydrogen phosphate, citric acid and sodium hydroxide.

73. The solid composition according to clause 72 wherein the aqueous composition additionally comprises poloxamer 338, sodium dihydrogen phosphate monohydrate, citric acid monohydrate and sodium hydroxide.

74. The solid composition according to any one of clauses 67-73, wherein the aqueous composition comprises from about 5 mg/mL to about 15 mg/mL of the poloxamer.

75. The solid composition according to any one of clauses 67-73, wherein the aqueous composition comprises from about 40 mg/mL to about 60 mg/mL of the poloxamer.

76. The solid composition according any one of the preceding clauses, wherein the aqueous composition additionally comprises an antioxidant, optionally wherein the antioxidant is methionine.

77. The solid composition according to clause 76, wherein the aqueous composition comprises from about 1.0 mg/mL to about 2.0 mg/mL of the antioxidant, for example about 1.5 mg/mL.

78. The solid composition according any one of the preceding clauses, wherein the aqueous composition does not comprise glucose.

79. The solid composition according to any one of the preceding clauses, wherein the aqueous composition has a pH of from about 5 to about 7.

80. The solid composition according to clause 79, wherein the aqueous composition has a pH of from about 6 to about 7.

81. The solid composition according to clause 80, wherein the aqueous composition has a pH of from about 6 to about 6.5.

82. The solid composition according to clause 81, wherein the aqueous composition has a pH of about 6.

83. A reconstituted aqueous composition obtainable by reconstituting the solid composition as defined in any one of clauses 1-82.

84. The reconstituted aqueous composition according to clause 83, wherein the reconstituting comprises adding an aqueous dispersion medium to the solid composition.

85. The reconstituted aqueous composition according to clause 84, wherein the aqueous dispersion medium is water.

86. A method for the treatment or prevention of HIV infection in a subject, the method comprising administering to the subject a reconstituted aqueous composition as defined in any one of clauses 83-85, in particular for the treatment of HIV infection in a subject.

87. A reconstituted aqueous composition as defined in any one of clauses 83-85 for use in the treatment or prevention of HIV infection in a subject, in particular for the treatment of HIV infection in a subject.

88. Use of a reconstituted aqueous composition as defined in any one of clauses 83-85 for the manufacture of a medicament for treating or preventing HIV infection in a subject, in particular for treating HIV infection in a subject.

89. The method, the reconstituted aqueous composition for use or the use according to clauses 86, 87 or 88, wherein the reconstituted aqueous composition is administered to the subject by intramuscular injection or subcutaneous injection.

90. The method, the reconstituted aqueous composition for use or the use according to clause 89, wherein the reconstituted aqueous composition is administered to the subject by intramuscular injection.

91. The method, the reconstituted aqueous composition for use or the use according to clause 89, wherein the reconstituted aqueous composition is administered to the subject by subcutaneous injection.

92. The method, the reconstituted aqueous composition for use or the use according to any one of clauses 86-91, wherein the reconstituted aqueous composition is administered to the subject intermittently at a time interval of about three months to about two years.

93. The method, the reconstituted aqueous composition for use or the use according to clause 92, wherein the time interval is about three months to about one year.

94. The method, the reconstituted aqueous composition for use or the use according to clause 93, wherein the time interval is about three months to about six months.

95. The method, the reconstituted aqueous composition for use or the use according to clause 93, wherein the time interval is about six months to about one year.

96. The method, the reconstituted aqueous composition for use or the use according to clause 94 or 95, wherein the time interval is about six months.

97. The method, the reconstituted aqueous composition for use or the use according to any one of clauses 86-96, wherein the HIV infection is HIV type 1 (HIV-1) infection.

98. The method, the reconstituted aqueous composition for use or the use according to any one of clauses 86-97, wherein the subject is a human.

99. A solid composition according to any one of clauses 1-82 for use in the treatment or prevention of HIV infection in a subject, in particular for the treatment of HIV infection in a subject.

100. Use of the solid composition according to any one of clauses 1-82 for the manufacture of a medicament for treating or preventing HIV infection in a subject, in particular for treating HIV infection in a subject.

101. A solid composition obtainable by freeze drying an aqueous composition comprising rilpivirine or a pharmaceutically acceptable salt thereof.

102. The solid composition according to clause 101, wherein the aqueous composition comprises rilpivirine or a pharmaceutically acceptable salt thereof in the form of particles suspended in the aqueous composition.

103. The solid composition according to clause 102, wherein the aqueous composition comprises rilpivirine or a pharmaceutically acceptable salt thereof in the form of micro- or nanoparticles suspended in the aqueous composition.

104. The solid composition according to clause 102 or 103, wherein the particles have a Dv90 of from about 100 nm to about 10 μm.

105. The solid composition according to clause 104, wherein the particles have a Dv90 of from about 500 nm to about 6 μm, optionally wherein the particles have a Dv90 of from about 500 nm to about 1,600 nm.

106. The solid composition according to clause 105, wherein the particles have a Dv90 of about 500 nm to about 700 nm.

107. The solid composition according to any one of clauses 102-106, wherein the particles have a Dv10 of from about 75 nm to about 200 nm.

108. The solid composition according to any one of clauses 102-107, wherein the particles have a Dv50 of from about 200 nm to about 500 nm.

109. The solid composition according to clause 102-104, wherein the particles have a Dv90 of from about 4 μm to about 6 μm.

110. The solid composition according to clause 109, wherein the particles have a Dv90 of about 5 μm to about 6 μm.

111. The solid composition according to any one of clauses 102-104 or 109, wherein the particles have a Dv10 of from about 300 nm to about 500 nm, and/or wherein the particles have a Dv50 of from about 1.5 μm to about 2 μm.

112. The solid composition according to any one of clauses 101-111, wherein the aqueous composition comprises from about 200 mg/mL to about 400 mg/mL rilpivirine or a pharmaceutically acceptable salt thereof.

113. The solid composition according to clause 112, wherein the aqueous composition comprises from about 250 mg/mL to about 350 mg/mL rilpivirine or a pharmaceutically acceptable salt thereof.

114. The solid composition according to clause 113, wherein the aqueous composition comprises about 300 mg/mL rilpivirine or a pharmaceutically acceptable salt thereof.

115. The solid composition according to any one of clauses 101-114, wherein the aqueous composition comprises rilpivirine, i.e. rilpivirine in free base form.

116. The solid composition according to any one of clauses 101-115, wherein the aqueous composition additionally comprises carboxymethyl cellulose or a pharmaceutically acceptable salt thereof.

117. The solid composition according to clause 116, wherein the carboxymethyl cellulose or a pharmaceutically acceptable salt thereof is sodium carboxymethyl cellulose.

118. The solid composition according to clause 116 or 117, wherein the carboxymethyl cellulose or a pharmaceutically acceptable salt thereof is not cross-linked.

119. The solid composition according to any one of clauses 116-118, wherein the carboxymethyl cellulose or a pharmaceutically acceptable salt thereof has a degree of saturation of carboxymethyl groups to cellulose of from about 0.5 to 1.

120. The solid composition according to any one of clauses 116-119, wherein the carboxymethyl cellulose or a pharmaceutically acceptable salt thereof has a molecular weight of from about 90 kDa to about 750 kDa, and/or wherein the carboxymethyl cellulose or a pharmaceutically acceptable salt thereof has a viscosity of from about 30 mPa·s to about 50 mPa·s.

121. The solid composition according to clause 116 or 120, wherein the aqueous composition comprises from about 1 mg/mL to about 100 mg/mL carboxymethyl cellulose or a pharmaceutically acceptable salt thereof.

122. The solid composition according to clause 121, wherein the aqueous composition comprises from about 1 mg/mL to about 50 mg/mL carboxymethyl cellulose or a pharmaceutically acceptable salt thereof.

123. The solid composition according to clause 122, wherein the aqueous composition comprises from about 1 mg/mL to about 5 mg/mL carboxymethyl cellulose or a pharmaceutically acceptable salt thereof.

124. The solid composition according to clause 123, wherein the aqueous composition comprises about 3 mg/mL carboxymethyl cellulose or a pharmaceutically acceptable salt thereof.

125. The solid composition according to clause 122, wherein the aqueous composition comprises from about 10 mg/mL to about 25 mg/mL carboxymethyl cellulose or a pharmaceutically acceptable salt thereof.

126. The solid composition according to any one of clauses 101-125, wherein the aqueous composition additionally comprises a cryoprotectant.

127. The solid composition according to clause 126, wherein the cryoprotectant is a sugar, sugar alcohol or an amino acid or a pharmaceutically acceptable salt thereof.

128. The solid composition according to clause 127, wherein the cryoprotectant is a sugar or sugar alcohol.

129. The solid composition according to clause 128, wherein the sugar or sugar alcohol is selected from mannitol and sucrose.

130. The solid composition according to clause 129, wherein the sugar or sugar alcohol is sucrose.

131. The solid composition according to clause 127, wherein the cryoprotectant is an amino acid or a pharmaceutically acceptable salt thereof.

132. The solid composition according to clause 131, wherein the amino acid or a pharmaceutically acceptable salt thereof is selected from arginine, glycine, and histidine or a pharmaceutically acceptable salt thereof, optionally wherein the amino acid or a pharmaceutically acceptable salt thereof is selected from arginine and glycine or a pharmaceutically acceptable salt thereof.

133. The solid composition according to clause 132, wherein the amino acid or a pharmaceutically acceptable salt thereof is arginine or a pharmaceutically acceptable salt thereof, for example arginine HCl.

134. The solid composition according to any one of clauses 126-133, wherein the aqueous composition comprises from about 1 mg/mL to about 200 mg/mL of the cryoprotectant.

135. The solid composition according to clause 134, wherein the aqueous composition comprises from about 1 mg/mL to about 150 mg/mL of the cryoprotectant.

136. The solid composition according to clause 135, wherein the aqueous composition comprises from about 30 mg/mL to about 100 mg/mL of the cryoprotectant.

137. The solid composition according to any one of clauses 127-130, wherein the aqueous composition comprises from about 25 mg/mL to about 125 mg/mL of the sugar or sugar alcohol.

138. The solid composition according to clause 137, wherein the aqueous composition comprises from about 75 mg/mL to about 125 mg/mL of the sugar or sugar alcohol.

139. The solid composition according to clause 138, wherein the aqueous composition comprises from about 50 mg/mL to about 100 mg/mL of the sugar or sugar alcohol.

140. The solid composition according to clause 139, wherein the aqueous composition comprises about 100 mg/mL of the sugar or sugar alcohol.

141. The solid composition according to any one of clauses 127 and 131-133, wherein the aqueous composition comprises from about 1 mg/mL to about 75 mg/mL of the amino acid or a pharmaceutically acceptable salt thereof.

142. The solid composition according to clause 141, wherein the aqueous composition comprises from about 1 mg/mL to about 50 mg/mL of the amino acid or a pharmaceutically acceptable salt thereof.

143. The solid composition according to clause 142, wherein the aqueous composition comprises from about 25 mg/mL to about 35 mg/mL of the amino acid or a pharmaceutically acceptable salt thereof, optionally wherein the aqueous composition comprises about 30 mg/mL of the amino acid or a pharmaceutically acceptable salt thereof.

144. The solid composition according to any one of clauses 126-143 wherein the ratio of rilpivirine or a pharmaceutically acceptable salt thereof to cryoprotectant in the aqueous composition is from about 1:1 (w/w) to about 20:1 (w/w).

145. The solid composition according to clause 144, wherein the ratio of rilpivirine or a pharmaceutically acceptable salt thereof to cryoprotectant in the aqueous composition is from about 2:1 (w/w) to about 10:1 (w/w), optionally wherein the ratio of rilpivirine or a pharmaceutically acceptable salt thereof to cryoprotectant in the aqueous composition is from about 3:1 (w/w) to about 10:1 (w/w).

146. The solid composition according to clause 144, wherein the ratio of rilpivirine or a pharmaceutically acceptable salt thereof to cryoprotectant in the aqueous composition is from about 2:1 (w/w) to about 7:1 (w/w).

147. The solid composition according to clause 146, wherein the ratio of rilpivirine or a pharmaceutically acceptable salt thereof to cryoprotectant in the aqueous composition is from about 3:1 (w/w) to about 6:1 (w/w).

148. The solid composition according to any one of clauses 126-147 wherein the ratio of carboxymethyl cellulose or a pharmaceutically acceptable salt thereof to cryoprotectant is from about 1:1 (w/w) to about 1:100 (w/w), optionally from about 1:1 (w/w) to about 1:50 (w/w).

149. The solid composition according to clause 148 wherein the ratio of carboxymethyl cellulose or a pharmaceutically acceptable salt thereof to cryoprotectant is from about 1:8 (w/w) to about 1:40 (w/w).

150. The solid composition according to any one of clauses-148 wherein the ratio of carboxymethyl cellulose or a pharmaceutically acceptable salt thereof to cryoprotectant is from about 1:2 (w/w) to about 1:15 (w/w).

151. The solid composition according to clause 150, wherein the ratio of carboxymethyl cellulose or a pharmaceutically acceptable salt thereof to cryoprotectant is from about 1:4 (w/w) to about 1:11 (w/w).

152. The solid composition according to clause 151, wherein the ratio of carboxymethyl cellulose or a pharmaceutically acceptable salt thereof to cryoprotectant is from about 1:5 (w/w) to about 1:10 (w/w).

153. The solid composition according to clause 151, wherein the ratio of carboxymethyl cellulose or a pharmaceutically acceptable salt thereof to cryoprotectant is from about 1:9 (w/w) to about 1:11 (w/w).

154. The solid composition according to clause 149, wherein the ratio of carboxymethyl cellulose or a pharmaceutically acceptable salt thereof to cryoprotectant is from about 1:30 (w/w) to about 1:35 (w/w).

155. The solid composition according to any one of clauses 101-154, wherein the aqueous composition additionally comprises a poloxamer, for example poloxamer 338.

156. The solid composition according to clause 155, when dependent on clause 102, wherein when the rilpivirine or a pharmaceutically acceptable salt thereof has a Dv90 of less than about 1600 nm the ratio of rilpivirine or a pharmaceutically acceptable salt thereof to poloxamer is from about 3:1 (w/w) to about 15:1 (w/w).

157. The solid composition according to clause 156, wherein when the rilpivirine or a pharmaceutically acceptable salt thereof has a Dv90 of less than about 1600 nm the ratio of rilpivirine or a pharmaceutically acceptable salt thereof to poloxamer is from about 6:1 (w/w) to about 15:1 (w/w).

158. The solid composition according to clause 157, wherein when the rilpivirine or a pharmaceutically acceptable salt thereof has a Dv90 of less than about 1600 nm the ratio of rilpivirine or a pharmaceutically acceptable salt thereof to poloxamer is about 6:1 (w/w).

159. The solid composition according to any one of clauses 155-158, wherein the relative amount (w/w) of rilpivirine or a pharmaceutically acceptable salt thereof to poloxamer is less than about 12:1 (w/w).

160. The solid composition according to any one of clauses 101-159, wherein the aqueous composition additionally comprises a poloxamer, sodium dihydrogen phosphate, citric acid and sodium hydroxide.

161. The solid composition according to clause-160, wherein the aqueous composition additionally comprises poloxamer 338, sodium dihydrogen phosphate monohydrate, citric acid monohydrate and sodium hydroxide.

162. The solid composition according to any one of clauses 160-161, wherein the aqueous composition comprises from about 5 mg/mL to about 15 mg/mL of the poloxamer.

163. The solid composition according to any one of clauses 160-161, wherein the aqueous composition comprises from about 40 mg/mL to about 60 mg/mL of the poloxamer.

164. The solid composition according to any one of clauses 101-163, wherein the aqueous composition additionally comprises an antioxidant, optionally wherein the antioxidant is methionine.

165. The solid composition according to clause 164, wherein the aqueous composition comprises from about 1.0 mg/mL to about 2.0 mg/mL of the antioxidant, for example about 1.5 mg/mL.

166. The solid composition according any one of clauses 101-165, wherein the aqueous composition does not comprise glucose.

167. The solid composition according to any one of clauses 101-166, wherein the aqueous composition has a pH of from about 5 to about 7.

168. The solid composition according to clause 167, wherein the aqueous composition has a pH of from about 6 to about 7.

169. The solid composition according to clause 168, wherein the aqueous composition has a pH of from about 6 to about 6.5.

170. The solid composition according to clause 169, wherein the aqueous composition has a pH of about 6.

171. The solid composition according to any one of clauses 101-170, wherein the aqueous composition does not comprise a hyaluronidase.

172. A reconstituted aqueous composition obtainable by reconstituting the solid composition as defined in any one of clauses 101-171.

173. The reconstituted aqueous composition according to clause 172, wherein the reconstituting comprises adding an aqueous dispersion medium to the solid composition.

174. The reconstituted aqueous composition according to clause 173, wherein the aqueous dispersion medium is water.

175. A method for the treatment or prevention of HIV infection in a subject, the method comprising administering to the subject a reconstituted aqueous composition as defined in any one of clauses 172-174, in particular for the treatment of HIV infection in a subject.

176. A reconstituted aqueous composition as defined in any one of clauses 172-174 for use in the treatment or prevention of HIV infection in a subject, in particular for the treatment of HIV infection in a subject.

177. Use of a reconstituted aqueous composition as defined in any one of clauses 172-174 for the manufacture of a medicament for treating or preventing HIV infection in a subject, in particular for treating HIV infection in a subject.

178. The method, the reconstituted aqueous composition for use or the use according to clauses 175-177, wherein the reconstituted aqueous composition is administered to the subject by intramuscular injection or subcutaneous injection.

179. The method, the reconstituted aqueous composition for use or the use according to clause 178, wherein the reconstituted aqueous composition is administered to the subject by intramuscular injection.

180. The method, the reconstituted aqueous composition for use or the use according to clause 178, wherein the reconstituted aqueous composition is administered to the subject by subcutaneous injection.

181. The method, the reconstituted aqueous composition for use or the use according to any one of clauses 175-180, wherein the reconstituted aqueous composition is administered to the subject intermittently at a time interval of about three months to about two years.

182. The method, the reconstituted aqueous composition for use or the use according to clause 181, wherein the time interval is about three months to about one year.

183. The method, the reconstituted aqueous composition for use or the use according to clause 182, wherein the time interval is about three months to about six months.

184. The method, the reconstituted aqueous composition for use or the use according to clause 182, wherein the time interval is about six months to about one year.

185. The method, the reconstituted aqueous composition for use or the use according to clause 183 or 184, wherein the time interval is about six months.

186. The method, the reconstituted aqueous composition for use or the use according to any one of clauses 175-185, wherein the HIV infection is HIV type 1 (HIV-1) infection.

187. The method, the reconstituted aqueous composition for use or the use according to any one of clauses 175-186, wherein the subject is a human.

188. A solid composition according to any one of clauses 101-171 for use in the treatment or prevention of HIV infection in a subject, in particular for the treatment of HIV infection in a subject.

189. Use of the solid composition according to any one of clauses 101-171 for the manufacture of a medicament for treating or preventing HIV infection in a subject, in particular for treating HIV infection in a subject.

Also described herein are the following numbered embodiments:

1. A solid composition obtainable by freeze drying an aqueous composition comprising rilpivirine or a pharmaceutically acceptable salt thereof and a hyaluronidase.

2. The solid composition according to embodiment 1, wherein the hyaluronidase is rHuPH20.

3. The solid composition according to any one of the preceding embodiments, wherein the aqueous composition comprises rilpivirine or a pharmaceutically acceptable salt thereof in the form of particles suspended in the aqueous composition, optionally wherein the particles are micro- or nanoparticles suspended in the aqueous composition.

4. The solid composition according to any one of the preceding embodiments, wherein the aqueous composition additionally comprises carboxymethyl cellulose or a pharmaceutically acceptable salt thereof.

5. The solid composition according to embodiment 4, wherein the carboxymethyl cellulose or a pharmaceutically acceptable salt thereof is sodium carboxymethyl cellulose.

6. The solid composition according to embodiment 4 or 5, wherein the carboxymethyl cellulose or a pharmaceutically acceptable salt thereof is not cross-linked.

7. The solid composition according to any one of embodiments 4-6, wherein the aqueous composition comprises from about 0.5 mg to about 2 mg carboxymethyl cellulose or a pharmaceutically acceptable salt thereof per 100 U hyaluronidase.

8. The solid composition according to any one of the preceding embodiments, wherein the aqueous composition additionally comprises a cryoprotectant.

9. The solid composition according to embodiment 8, wherein the cryoprotectant is a sugar, sugar alcohol or an amino acid or a pharmaceutically acceptable salt thereof.

10. The solid composition according to embodiment 9, wherein the sugar or sugar alcohol is mannitol or sucrose and the amino acid or a pharmaceutically acceptable salt thereof is arginine or glycine.

11. The solid composition according to any one of embodiments 8-10, wherein the ratio of rilpivirine or a pharmaceutically acceptable salt thereof to cryoprotectant in the aqueous composition is from about 2:1 (w/w) to about 7:1 (w/w).

12. The solid composition according to anyone of embodiments 8-11, wherein the ratio of carboxymethyl cellulose or a pharmaceutically acceptable salt thereof to cryoprotectant is from about 1:4 (w/w) to about 1:11 (w/w).

13. The solid composition according to any one of the preceding embodiments, wherein the aqueous composition has a pH of from about 6 to about 6.5.

14. A reconstituted aqueous composition obtainable by reconstituting the solid composition as defined in any one of embodiments 1-13 with an aqueous dispersion medium.

15. A method for the treatment or prevention of HIV infection in a subject, the method comprising administering to the subject a reconstituted aqueous composition as defined in embodiment 14.

1. A solid composition obtainable by freeze drying an aqueous composition comprising rilpivirine or a pharmaceutically acceptable salt thereof and a hyaluronidase.

2. The solid composition according to claim 1, wherein the hyaluronidase is rHuPH20.

3. The solid composition according to claim 1, wherein the aqueous composition comprises rilpivirine or a pharmaceutically acceptable salt thereof in the form of particles suspended in the aqueous composition, optionally wherein the particles are micro- or nanoparticles suspended in the aqueous composition.

4. The solid composition according to claim 1, wherein the aqueous composition additionally comprises carboxymethyl cellulose or a pharmaceutically acceptable salt thereof.

5. The solid composition according to claim 4, wherein the carboxymethyl cellulose or a pharmaceutically acceptable salt thereof is sodium carboxymethyl cellulose.

6. The solid composition according to claim 4, wherein the carboxymethyl cellulose or a pharmaceutically acceptable salt thereof is not cross-linked.

7. The solid composition according to claim 4, wherein the aqueous composition comprises from about 0.05 mg to about 0.25 mg carboxymethyl cellulose or a pharmaceutically acceptable salt thereof per 100 U hyaluronidase.

8. The solid composition according to claim 1, wherein the aqueous composition additionally comprises a cryoprotectant.

9. The solid composition according to claim 8, wherein the cryoprotectant is a sugar, sugar alcohol or an amino acid or a pharmaceutically acceptable salt thereof.

10. The solid composition according to claim 9, wherein the sugar or sugar alcohol is sucrose and the amino acid or a pharmaceutically acceptable salt thereof is arginine or a pharmaceutically acceptable salt thereof.

11. The solid composition according to claim 8, wherein the ratio of rilpivirine or a pharmaceutically acceptable salt thereof to cryoprotectant in the aqueous composition is from about 1:1 (w/w) to about 20:1 (w/w).

12. The solid composition according to claim 8, wherein the ratio of carboxymethyl cellulose or a pharmaceutically acceptable salt thereof to cryoprotectant is from about 1:8 (w/w) to about 1:40 (w/w).

13. The solid composition according to claim 1, wherein the aqueous composition has a pH of from about 6 to about 6.5.

14. A reconstituted aqueous composition obtainable by reconstituting the solid composition as defined in claim 1 with an aqueous dispersion medium.

15. A method for the treatment or prevention of HIV infection in a subject, the method comprising administering to the subject a reconstituted aqueous composition as defined in claim 14.

16. The solid composition according to claim 10, wherein the amino acid or a pharmaceutically acceptable salt thereof is arginine HCl.
